SEQUENCE LISTING (47 sequences)

SEQ ID NO: 1 - DNA, 1425 bp, Macaca fascicularis
atggccaagcttaccctcctcactggtctgctccttgtgctgacagctgagataggctctgcttaccagctgacatgctacttctccaactgggcccagtaccggccaggcctggggcgcttcatgcctgacgacatcgacccctgcctctgtacccacctgatctacgcctttgctgggatgcagaacaacaagatcaccaccatcgaatggaatgacgtgactctctaccaagctttcaatggcctgaaaaacaaaaatagtcaactgaaaactctcttggctattggtggctggaactttggcactgctcctttcactgccatggtttccactcctgcgaaccgccagactttcatcaactcagtcatcgaattcctgcgccagtatgagtttgatgggctggacttcgactgggagtaccccggctctcgtgggagcccttctcaggacaagcatctcttcactgtcctggtgcaggaaacgcgtgaagcttttgagcaggaggccaagcagagcaacaagcccaggctgctggtcactgctgcagtagctgctggcatctccaccatccaatctggctacgagatcccccaactgtcgcagtacctggactacatccacgtcatgacctacgacctccatggcccctgggagggctacacaggagagaacagccccctgtacaaatacccgactgacactggcagtaacgcctacctcaatgtggattatgccatcaactactggaagaacaacggggccccagctgagaagctcatcgttggattcccagcctatggacataccttccttctgagcaatccctccgatcatggaattggcgcccctaccactggtcctggccctgctgggccctataccagggagtctgggttctgggcctactatgagatctgtaccttcctgaagaatggagctactgaagtatgggaggctaatgaggatgttccctatgcctacaaaggaaatgagtggcttggatatgataacaccaagagtttccaaatcaaggctgactggctaaagaagaacaacttcggaggcgccatggtctgggccattgacttggatgatttcacaggcactttctgcaatgaaggaaaattccctctgatcaccaccctaaaggatgctctgggcctgcagagtacaagttgcaaagctccagcccaacccattactcccattactgaggcacccgtcacaggcagcgtcagccacagtggtagctctgggggcagccccagtgatagtgaattctgtgccaacaaggccaatggcctgtaccctgaccccactgacaagaatgccttctacaattgtgcaaatggaaagactttcattcagcactgccaggctggccttgtcttcgaagcctcctgctcctgctgcagctggtga

SEQ ID NO: 2 - protein, 474 aa, Macaca fascicularis [sequence omitted]

SEQ ID NO: 3 - DNA, 1449 bp, Homo sapiens
atggccaagctcaccctcctcactggtctgctccttgtgctgacagctgaaataggctctgcctaccagctgacatgttacttcaccaactgggcccagaaccagccaggcctggggtgcttcaagcctgatgacatcgacccctgcctctgtacccacttgatctacgcctttgctggaatgcagaacaacgagatcaccaccatcgaatgggatgacatgactctctaccaagctttcaatggcctgaaaaacaagaatagtcaactgaaaactctcttggctattggtggctggaactttggcactgctccgttcactgccatggtttccactcctgagaaccaccagactttcatcaactcagtcatcaaattcctgcgccagtatgagtttgacgggctggactttgactgggagtaccccggctctcgtgtgagccctcctcaggacaagcatctcttcactgtcctggtgcaggaaatgcgtgaagcttttgagcaggaggccaagcacattaataagcccaggctgatggtcactgctgcagtagctgctggcatctccaacatccagtctggctatgagatcccccaactgtcacagtacccggactacatccatgtcatgacctatgacctccatggctcctgggagggctacactggagagaacagccccctctacaaatacccgactgacaccggcagcaacgcctacctcaatgtggattatgtcatgaactactggaaggacaacagggccccagctgagaagctcttggttggattcccagcctatggacactccttccttctgagcaacccctccaaccatggaattgatgcccctaccactggtcctggccctgctggaccctataccaggcagtctgggttctgggcctactatgagatctgtaccttcctgaagaatggagctactgaagtatgggaggcttctgaggatgttccctatgcctacaaaggaaatgagtggcttggatacgataacaccaagagtttccaaatcaaggcagattggctaaagaagaacaactttggaggtgccatggtctgggccattgacctggatgatttcacaggcactttctgcaaccaaggaaaattccctctgatcaccaccctgaaggatgctctgggcctgcagagtacaagttgcaaagctccagcccaacccattgctcccattgccgaggcaaacatcacatgcggtgtcagccacagtggtagctctgggggccgctctggcaggagctctgggggcagccccagaggtagtggattctgtgctgacagggccagtggcctgtaccctgaccccactgacaagaatgcctcctacagttgtgtgaatggaaagactttcactcagcactgccagcctggtggtgtctttgataccttctgctcctgctgcagctggtga

SEQ ID NO: 4 - protein, 482 aa, Homo sapiens
MAKLTLLTGLLLVLTAEIGSAYQLTCYFTNWAQNQPGLGCFKPDDIDPCLCTHLIYAFAGMQNNEITTIEWDDMTLYQAFNGLKNKNSQLKTLLAIGGWNFGTAPFTAMVSTPENHQTFINSVIKFLRQYEFDGLDFDWEYPGSRVSPPQDKHLFTVLVQEMREAFEQEAKHINKPRLMVTAAVAAGISNIQSGYEIPQLSQYPDYIHVMTYDLHGSWEGYTGENSPLYKYPTDTGSNAYLNVDYVMNYWKDNRAPAEKLLVGFPAYGHSFLLSNPSNHGIDAPTTGPGPAGPYTRQSGFWAYYEICTFLKNGATEVWEASEDVPYAYKGNEWLGYDNTKSFQIKADWLKKNNFGGAMVWAIDLDDFTGTFCNQGKFPLITTLKDALGLQSTSCKAPAQPIAPIAEANITCGVSHSGSSGGRSGRSSGGSPRGSGFCADRASGLYPDPTDKNASYSCVNGKTFTQHCQPGGVFDTFCSCCSW

SEQ ID NO: 5 - DNA, 25 bp, Artificial Sequence
  Description of Artificial Sequence Synthetic primer
tcagaacatataaaaagctctgcgg

SEQ ID NO: 6 - DNA, 27 bp, Artificial Sequence
  Description of Artificial Sequence Synthetic primer
ctctagggaatatagaccaggtcaggt

SEQ ID NO: 7 - DNA, 24 bp, Artificial Sequence
  Description of Artificial Sequence Synthetic primer
tataaatggcaggttggatgaggg

SEQ ID NO: 8 - DNA, 26 bp, Artificial Sequence
  Description of Artificial Sequence Synthetic primer
ctgggtgaggtgatatctgaaaaatg

SEQ ID NO: 9 - DNA, 23 bp, Artificial Sequence
  Description of Artificial Sequence Synthetic primer
ttctcctcacaggtcttgtcctt

SEQ ID NO: 10 - DNA, 23 bp, Artificial Sequence
  Description of Artificial Sequence Synthetic primer
ccagttggtgaagtagcatgtca

SEQ ID NO: 11 - DNA, 26 bp, Artificial Sequence
  Description of Artificial Sequence Synthetic primer
atttgcagctcggctctgcctaccag

SEQ ID NO: 12 - DNA, 21 bp, Artificial Sequence
  Description of Artificial Sequence Synthetic primer
gcataaggcacttcctgaggg

SEQ ID NO: 13 - DNA, 21 bp, Artificial Sequence
  Description of Artificial Sequence Synthetic primer
tgacaacatcaagagcttcga

SEQ ID NO: 14 - DNA, 23 bp, Artificial Sequence
  Description of Artificial Sequence Synthetic primer
agccaacccacacattgccctga

SEQ ID NO: 15 - DNA, 20 bp, Artificial Sequence
  Description of Artificial Sequence Synthetic primer
ttctgttatttgccacgggg

SEQ ID NO: 16 - DNA, 16 bp, Artificial Sequence
  Description of Artificial Sequence Synthetic primer
cccggcctggcagttc

SEQ ID NO: 17 - DNA, 25 bp, Artificial Sequence
  Description of Artificial Sequence Synthetic primer
ccttctggcactgcgtgaatggagt

SEQ ID NO: 18 - DNA, 21 bp, Artificial Sequence
  Description of Artificial Sequence Synthetic primer
acaaggagcagaccagtgagg

SEQ ID NO: 19 - DNA, 24 bp, Artificial Sequence
  Description of Artificial Sequence Synthetic primer
tgacatgttacttcaccaactggg

SEQ ID NO: 20 - DNA, 30 bp, Artificial Sequence
  Description of Artificial Sequence Synthetic primer
ctggtaggcagagcctatttcagctgtcag

SEQ ID NO: 21 - DNA, 17 bp, Artificial Sequence
  Description of Artificial Sequence Synthetic primer
cagggccccagctgaga

SEQ ID NO: 22 - DNA, 21 bp, Artificial Sequence
  Description of Artificial Sequence Synthetic primer
gggttgctcagaaggaaggag

SEQ ID NO: 23 - DNA, 28 bp, Artificial Sequence
  Description of Artificial Sequence Synthetic primer
ctcttggttggattcccagcctatggac

SEQ ID NO: 24 - DNA, 25 bp, Artificial Sequence
  Description of Artificial Sequence Synthetic primer
tcctgaagaatggagctactgaagt

SEQ ID NO: 25 - DNA, 21 bp, Artificial Sequence
  Description of Artificial Sequence Synthetic primer
ccaagccactcatttcctttg

SEQ ID NO: 26 - DNA, 28 bp, Artificial Sequence
  Description of Artificial Sequence Synthetic primer
tgggaggcttctgaggatgttccctatg

SEQ ID NO: 27 - DNA, 60 bp, Artificial Sequence
  Description of Artificial Sequence Synthetic primer
ggggacaagtttgtacaaaaaagcaggcttccaccatggccaagcttaccctcctcactg

SEQ ID NO: 28 - DNA, 75 bp, Artificial Sequence
  Description of Artificial Sequence Synthetic primer
ggggaccactttgtacaagaaagctgggtcctagtgatggtgatggtgatgccagctgcagcaggagcagaaggt

SEQ ID NO: 29 - protein, 476 aa, Homo sapiens
MTKLILLTGLVLILNLQLGSAYQLTCYFTNWAQYRPGLGRFMPDDINPCLCTHLIYAFAGMQNNEITTIEWNDVTLYQAFNGLKNKNSQLKTLLAIGGWNFGTAPFTAMVSTPENRQTFITSVIKFLRQYEFDGLDFDWEYPGSRGSPPQDKHLFTVLVQEMREAFEQEAKQINKPRLMVTAAVAAGISNIQSGYEIPQLSQYLDYIHVMTYDLHGSWEGYTGENSPLYKYPTDTGSNAYLNVDYVMNYWKDNGAPAEKLIVGFPTYGHNFILSNPSNTGIGAPTSGAGPAGPYAKESGIWAYYEICTFLKNGATQGWDAPQEVPYAYQGNVWVGYDNVKSFDIKAQWLKHNKSGGAMVWAIDLDDFTGTFCNQGKFPLISTLKKALGLQSASCTAPAQPIEPITAAPSGSGNGSGSSSSGGSSGGSGFCAGRANGLYPVANNRNAFWHCVNGVTYQQNCQAGLVFDTSCDCCNWA

SEQ ID NO: 30 - DNA, 56 bp, Artificial Sequence
  Description of Artificial Sequence Synthetic oligonucleotide
gctttcaatggcctgaaaaacaagaatagtcaactgaaaactctcttggctattgg

SEQ ID NO: 31 - DNA, 56 bp, Artificial Sequence
  Description of Artificial Sequence Synthetic oligonucleotide
ccaatagccaagagagttttcagttgactattcttgtttttcaggccattgaaagc

SEQ ID NO: 32 - DNA, 42 bp, Artificial Sequence
  Description of Artificial Sequence Synthetic oligonucleotide
ccccagctgagaagctcttggttggattcccagcctatggac

SEQ ID NO: 33 - DNA, 42 bp, Artificial Sequence
  Description of Artificial Sequence Synthetic oligonucleotide
gtccataggctgggaatccaaccaagagcttctcagctgggg

SEQ ID NO: 34 - DNA, 36 bp, Artificial Sequence
  Description of Artificial Sequence Synthetic oligonucleotide
gcggtgtcagccacagtggtagctctgggggccgct

SEQ ID NO: 35 - DNA, 36 bp, Artificial Sequence
  Description of Artificial Sequence Synthetic oligonucleotide
agcggcccccagagctaccactgtggctgacaccgc

SEQ ID NO: 36 - protein, 8 aa, Artificial Sequence
  Description of Artificial Sequence Synthetic peptide
DGXDXDXE

SEQ ID NO: 37 - DNA, 94 bp, Homo sapiens
gaagcctttgtgataaccacagaatcagaacatataaaaagctctgcgggactggtgctgactgcaaccatgacaaagcttattctcctcacag

SEQ ID NO: 38 - DNA, 68 bp, Homo sapiens
tataaatggcaggttggatgagggacactaccactgaggcaacatggccaagctcacccttctcactg

SEQ ID NO: 39 - DNA, 120 bp, Homo sapiens
tcgacaccagctgtgattgctgcaactgggcataaacctgacctggtctatattccctagagttccagtctcttttgcttaggacatgttgcccctacctaaagtcctgcaataaaatca

SEQ ID NO: 40 - DNA, 87 bp, Homo sapiens
ttgataccttctgctcctgctgcagctggtgataacatttttcagatatcacctcacccagcctcagaagttgttgtgcaataaaag

SEQ ID NO: 41 - DNA, 1448 bp, Homo sapiens
atggccaagctcacccttctcactggtctgctccttgtgctgacagctgaaataggctctgcctaccagctgacatgttacttcaccaactgggcccagaaccagccaggcctggggtgcttcaagcctgatgacatcgacccctgcctctgtacccacttgatctacgcctttgctggaatgcagaacaacgagatcaccaccatcgaatgggatgacatgactctctaccaagctttcaatggcctgaaaaacaagaatagtcaactgaaaactctcttggctattggtggctggaactttggcactgctcctttcactgccatggtttccactcctgagaaccaccagactttcatcaactcagtcatcaaattcctgcgccagtatgagtttgacgggctggactttgactgggagtaccccggctctcgtgtgagccctcctcaggacaagcatctcttcactgtcctggtgcaggaaatgcgtgaagcttttgagcaggaggccaagcacattaataagcccaggctgatggtcactgctgcagtagctgctggcatctccaacatccagtctggctatgagatcccccaactgtcacagtacccggactacatccatgtcatgacctatgacctccatggctcctgggagggctacactggagagaacagccccctctacaaatacccgactgacaccggcagcaacgcctacctcaatgtggattatgtcatgaactactggaaggacaacagggccccagctgagaagctcatgttggattcccagcctatggacactccttccttctgagcaacccctccaaccatggaattgatgcccctaccactggtcctggccctgctggaccctataccaggcagtctgggttctgggcctactatgagatctgtaccttcctgaagaatggagctactgaagtatgggaggcttctgaggatgttccctatgcctacaaaggaaatgagtggcttggatacgataacaccaagagtttccaaatcaaggcagattggctaaagaagaacaactttggaggtgccatggtctgggccattgacctggatgatttcacaggcactttctgcaaccaaggaaaattccctctgatcaccaccctgaaggatgctctgggcctgcagagtacaagttgcaaagctccagcccaacccattgctcccattgccgaggcaaacatcacatgcggtgtcagccacagtcgtagctctgggggccgctctggcaggagctctgggggcagccccagaggtagtggattctgtgctgacagggccagtggcctgtaccctgaccccactgacaagaatgcctcctacagttgtgtgaatggaaagactttcactcagcactgccagcctggtggtgtcttttataccttctgctcctgctgcagctggtga

SEQ ID NO: 42 - protein, 272 aa, Homo sapiens
MAKLTLLTGLLLVLTAEIGSAYQLTCYFTNWAQNQPGLGCFKPDDIDPCLCTHLIYAFAGMQNNEITTIEWDDMTLYQAFNGLKNKNSQLKTLLAIGGWNFGTAPFTAMVSTPENHQTFINSVIKFLRQYEFDGLDFDWEYPGSRVSPPQDKHLFTVLVQEMREAFEQEAKHINKPRLMVTAAVAAGISNIQSGYEIPQLSQYPDYIHVMTYDLHGSWEGYTGENSPLYKYPTDTGSNAYLNVDYVMNYWKDNRAPAEKLMLDSQPMDTPSF

SEQ ID NO: 43 - protein, 36 aa, Homo sapiens
ATPPTMELMPLPLVLALLDPIPGSLGSGPTMRSVPS

SEQ ID NO: 44 - protein, 38 aa, Homo sapiens
RMELLKYGRLLRMFPMPTKEMSGLDTITPRVSKSRQIG

SEQ ID NO: 45 - protein, 29 aa, Homo sapiens
RRTTLEVPWSGPLTWMISQALSATKENSL

SEQ ID NO: 46 - protein, 74 aa, Homo sapiens
RMLWACRVQVAKLQPNPLLPLPRQTSHAVSATVVALGAALAGALGAAPEVVDSVLTGPVACTLTPLTRMPPTVV

SEQ ID NO: 47 - protein, 24 aa, Homo sapiens [sequence omitted]

(19) United States
(12) Patent Application Publication
(10) Pub. No.: US 2009/0010942 A1
(43) Pub. Date: Jan. 8, 2009

(54) NOVEL PRIMATE CHITINASES AND USES THEREFOR

(75) Inventors: Rustem KRYKBAEV, Brookline, MA (US); Padmalatha S. REDDY, Lexington, MA (US); Aaron WINKLER, Arlington, MA (US); Lori J. FITZ, Somerville, MA (US)

Correspondence Address: LOWRIE, LANDO & ANASTASI, LLP;W2023, ONE MAIN STREET, SUITE 1100, CAMBRIDGE, MA, 02142, US

(73) Assignee: WYETH, Madison, NJ (US)

(21) Appl. No.: 12/123,111

(22) Filed: May 19, 2008

Related U.S. Application Data
(60) Provisional application No. 60/930,777, filed on May 18, 2007.

(51) Int. Cl. A61K 39/395; C12N 15/56; C12N 5/10; G01N 33/53; A61P 29/00; C12Q 1/68; C07K 14/00; C12P 21/04

(52) U.S. Cl. 424/158.1; 536/23.2; 435/325; 530/350; 435/71.1; 435/7.1; 435/6; 435/7.71

(57) ABSTRACT

Novel primate chitinases (pNC) and nucleic acids encoding these chitinases are disclosed. The pNCs of the invention are homologous to human acidic mammalian chitinases (hAMCase) and human chitotriosidase, in terms of their exon/intron genomic structure and conserved catalytic sites. The polypeptides and nucleic acids of the invention can be used to develop, and/or screen for, modulators of chitinase-activities, e.g., antisense, antibodies, RNAi. The modulators identified using methods and reagents disclosed herein can be used to reduce chitinase-associated inflammatory conditions.

### CROSS REFERENCE TO RELATED APPLICATIONS

This application claims priority to U.S. Ser. No. 60/930,777 filed on May 18, 2007. The contents of the aforementioned application are hereby incorporated by reference in their entirety. This application also incorporates by reference the International Application filed with the U.S. Receiving Office on May 19, 2008, entitled “Novel Primate Chitinases and Uses Therefor” and bearing attorney docket number W2023-7008WO.

### SEQUENCE LISTING

An electronic copy of the Sequence Listing in both pdf and txt formats is being submitted herewith.

### BACKGROUND

Chitins, a polymer of β-1,4-N-acetyl-glucosamine (GlcNAc), are abundantly produced in nature in the exoskeleton of crustaceans and insects, the sheath of nematodes, the cell wall of fungi, and other marine organisms, parasites, and pathogens; but not by mammals. Chitin is degraded by chitinases (EC 3.2.1.14) that belong to members of the glycohydrolase family 18. This family is characterized by an eight fold α/β barrel structure and includes bacterial, as well as plant chitinases. Due to the lack of chitin in mammalian bodies, it has been generally assumed that chitinases are not present in mammals. Recent studies, however, have identified chitinases and chitinase-like proteins (CLPs) belonging to the glycohydrolase family 18 in mice and human, including, chitinase 3-like-1 (CHI3L1), chitotriosidase, YKL-39, Yml, acidic mammalian chitinase (AMCase), oviduct-specific glycoprotein, and stabilin-1-interacting chitinase-like protein (Hakala, et al., J. Biol. Chem., 268:25803-25810, 1993; Boot, et al., J. Biol. Chem., 270:26252-26256, 1995; Hu, et al., J. Biol. Chem., 271:19415-19420, 1996; Jin, et al., Genomics, 54:316-322, 1998; Boot, et al., J. Biol. Chem., 276:6770-6778, 2001; Buhi, W. C., Reproduction, 123:355-362, 2002; Kzhshkowska, et al., Blood, 107:3221-3228, 2006; and Chang, et al., J. Biol. Chem., 276:17497-17506, 2001). Chitotriosidase and AMCase possess chitinase enzymatic activity, whereas other currently identified mammalian chitinases, including CLPs, do not possess this activity (Chang, et al., J. Biol. Chem., 276:17497-17506, 2001). The consensus catalytic site in enzymatically active chitinases is DGXDXDXE (SEQ ID NO:36) on strand β4. Within this motif, the catalytic activity is mediated by glutamic acid (E), which protonates the glycosidic bond with chitin (van Aalten, et al., Proc. Natl. Acad. Sci. USA., 98:8979-8984, 2001).

The prototypic chitinase, AMCase, catalyses the hydrolysis of artificial chitin-like substrates. It is unique among mammalian enzymes in that it has an acidic pH optimum. For example, AMCase catalyzes the conversion of chitin to N-acetyl-D-glucosamine. AMCase has been shown to be expressed predominately in the gastrointestinal tract, particularly the stomach, and to a lesser extent in the lung and is thought to play a role in digestion or possibly anti-parasitic defense mechanism.

AMCase is a secreted enzyme about 52.2 kD encoded by chromosome 1p13.1-p21.3 (Boot, et al., J. Biol. Chem., 270:26252-26256, 1995; Saito A., et al., Gene, 239:325-331, 1999). Optimum AMCase activity is seen at pH 4-5, and it is typically found in stomach, salivary gland, and lung. AMCase is induced during T_{H}2 inflammation through an IL-13-dependent mechanism (Elias J. A., et al., J. Allergy Clin. Immunol., 116:497-500, 2005; Elias J. A., et al., J. Clin. Invest., 111:291-297, 2003). Moreover, the AMCase mRNA is significantly increased in an animal model of asthma compared to control, non-asthmatic animals (Zhu Z., et al., Science, 304:1678-1682, 2004). The mRNA encoding AMCase protein is elevated by intrathecal ovalbumin challenge or direct pulmonary instillation of IL-13, and blocking binding of IL-13 to the IL-13 receptor inhibits AMCase expression (Zhu Z., et al., Science, 304:1678-1682, 2004). AMCase neutralization has been shown to reduce asthmatic pathology in animal models; for example, lowered BAL inflammation has been observed in a mouse asthma model and reduced airway hyperactivity and dynamic compliance has been detected in OVA models in response to AMCase inhibition (WO 2004/092404).

Given the important biological activities of chitinases in normal and pathological conditions such as asthma, chronic obstructive pulmonary disease (COPD), and other inflammatory conditions, there exists a need for the identification of novel chitinase genes, as well as for the discovery of modulators of chitinase activity for use in regulating a variety of normal and/or pathological cellular processes.

### SUMMARY

The present invention is based, at least in part, on the discovery of novel primate chitinases and nucleic acids encoding these chitinases, referred to herein collectively as “primate Novel Chitinases” or “pNCs,” or by their individual names “macaque Novel Chitinase” or “mNC,” and “human Novel Chitinase” or “hNC.” The pNCs of the invention are homologous to human acidic mammalian chitinases (hAMCase), in terms of their exon/intron genomic structure and conserved catalytic sites. The nucleotide sequence of a cDNA encoding mNC is shown in FIG. 1 (SEQ ID NO: 1), and the amino acid sequence of an mNC polypeptide is shown in FIG. 2 (SEQ ID NO:2). The nucleotide sequence of a cDNA encoding hNC, which is an engineered form of, is shown in FIG. 3A (SEQ ID NO:3), and the amino acid sequence is shown in FIG. 4A (SEQ ID NO:4). Chitinases have been associated with a variety of inflammatory conditions, such as inflammatory pulmonary conditions (e.g., asthma, COPD, emphysema), scleroderma, allergy, and inflammatory bowel disease. Accordingly, the pNC polypeptides and nucleic acids of the invention can be used, for example, as targets in assays applicable for treating and/or diagnosing chitinase-associated disorders. For example, the pNC polypeptides and nucleic acids can be used to develop, and/or screen for, modulators (e.g., inhibitors) of CLP-activities, e.g., antisense, antibodies and RNAi. The modulators identified using the pNC polypeptides and nucleic acids disclosed herein can be used to reduce chitinase activities associated with inflammatory conditions.

Accordingly, in one aspect, the invention features an isolated nucleic acid molecule that encodes a pNC protein or polypeptide, e.g., a biologically active fragment thereof (e.g., an mNC fragment comprising, or consisting of, a chitinase catalytic domain (e.g., about amino acids 22 to 408 of SEQ ID NO:2), a linker (e.g., about amino acids 409 to 427 of SEQ ID NO:2), and/or a chitin-binding domain (e.g., about amino acids 428 to 474 of SEQ ID NO:2). In one embodiment, the isolated nucleic acid molecule encodes a polypeptide comprising, or consisting of, the amino acid sequence of SEQ ID NO:2 or SEQ ID NO:4, or an amino acid sequence substantially identical thereto (e.g., an amino acid sequence about 83%, 84%, 85%, 87%, 90%, 95%, 98% or more identical to the amino acid sequence of SEQ ID NO:2 or SEQ ID NO:4), or a fragment thereof (e.g., a biologically active fragment thereof as described herein). In some embodiments, the encoded polypeptide possesses the chitin-binding activity and/or chitinase catalytic activity of SEQ ID NO:2 or 4. In other embodiments, the invention provides isolated pNC nucleic acid molecules comprising, or consisting of, the nucleotide sequence shown in SEQ ID NO:1 or SEQ ID NO:3, or a sequence substantially identical thereto (e.g., a nucleotide sequence about 85%, 87%, 90%, 95%, 98% or more identical to the nucleotide sequence shown in SEQ ID NO:1 or SEQ ID NO:3), or a fragment thereof. In some embodiments, the encoded polypeptide possesses the chitin-binding activity and/or chitinase catalytic activity of a polypeptide encoded by SEQ ID NO:1 or 3. In embodiments, the pNC nucleic acid is a fragment of at least about 100, 300, 350, 400, 450, 500, 600, 700, 800, 900, 1000, 1100, 1200 or more contiguous nucleotides of SEQ ID NO:1 or SEQ ID NO:3. In other embodiments, the invention provides a nucleic acid molecule which hybridizes under a stringent hybridization condition described herein to a nucleic acid molecule comprising the nucleotide sequence of SEQ ID NO:1 or SEQ ID NO:3 (or a complement thereof). In embodiments, the nucleic acid molecule that hybridizes to the complements of SEQ ID NO:3 or SEQ ID NO:1 encodes a full length pNC protein or an active fragment thereof (e.g., a fragment as described herein). In some embodiments, the encoded polypeptide possesses the chitin-binding activity and/or chitinase catalytic activity of a polypeptide encoded by SEQ ID NO:1 or 3.

In a related aspect, the invention further provides nucleic acid constructs that include a pNC nucleic acid molecule described herein. In certain embodiments, the nucleic acid molecules of the invention are operatively linked to native or heterologous regulatory sequences. Also included are vectors and host cells containing the pNC nucleic acid molecules of the invention, e.g., vectors and host cells suitable for producing pNC nucleic acid molecules and polypeptides.

In another related aspect, the invention provides nucleic acid fragments suitable as primers or hybridization probes for the amplification and/or detection of pNC-encoding nucleic acids. In embodiments, the primers or hybridization probes distinguish between the pNCs disclosed herein, or the sequences disclosed herein and chitinases from other species, e.g., human AMCase or human chitotriosidase. The nucleic acid fragments of the invention can also be used to distinguish between a chitinase nucleotide sequence encoding a functional or a non-functional chitinase, e.g., between a functional chitinase and a truncated or a pseudogene chitinase. Exemplary nucleotide sequences that can be used to detect specifically the pNC-encoding nucleic acids include the following nucleotides: about nucleotides 513-524, 780-784, 830-835, 843-847, 1117-1124, 1211-1237, 1211-1226, 1220-1237, 1262-1288, 1295-1305, 1312-1320, 1360-1376, 1404-1430, 1404-1414 and 1418-1430, of SEQ ID NO:1 or SEQ ID NO: 3, or a complementary nucleotide sequence thereof. Examples of primers or probes that can be used as disclosed herein, e.g., as SEQ ID NOs: 30-35.

In still another related aspect, isolated nucleic acid molecules that are nucleic acid inhibitors, e.g., antisense, RNAi, of a pNC-encoding nucleic acid molecule are provided. Exemplary nucleotide sequences that can be used to specifically inhibit expression of the pNC-encoding nucleic acids include the following nucleotides: about nucleotides 513-524, 780-784, 830-835, 843-847, 1117-1124, 1211-1237, 1211-1226, 1220-1237, 1262-1288, 1295-1305, 1312-1320, 1360-1376, 1404-1430, 1404-1414 and 1418-1430, of SEQ ID NO:1 or SEQ ID NO: 3, or a complementary nucleotide sequence thereof.

In other embodiments, the invention provides an isolated or purified pNC protein or polypeptide, e.g., a biologically active or antigenic fragment thereof (e.g., an mNC portion comprising, or consisting of, a chitinase catalytic domain (e.g., about amino acids 22 to 408 of SEQ ID NO:2), a linker (e.g., about amino acids 409 to 427 of SEQ ID NO:2), and/or a chitin-binding domain (e.g., about amino acids 428 to 474 of SEQ ID NO:2). The pNC can be a mature pNC protein or an unprocessed full length pNC protein (including the signal sequence). In one embodiment, the pNC polypeptide or fragment thereof includes at least one, two, three, five, ten, or twenty amino acid residues that differ from a human AMCase or a human chitotriosidase. In some embodiments, the polypeptide possesses the chitin-binding activity and/or chitinase catalytic activity of SEQ ID NO:2 or 4. Also featured are related pNC polypeptides that differ from human AMCase at one or more of amino acid residues 114, 149, 162, 173, 190, 217, 246, 266, 270, 279, 288, 295, 300, 317, 321, 332, 339, 343, 347, 351, 353, 354, 385, 392, 395, 402, 406, 409, 410, 414, 416, 418, 424, 425, 426, 428, 432, 440, 441, 442, 451, 454, 457, 468, or 471 of the human AMCase sequence shown in FIG. 12A (SEQ ID NO:29). In certain embodiments the differences include one or more conservative substitutions (e.g., up to 1, 2, 3, 4, 5, 6, 7, 8, 9, 10, 11, 12, 13, 14, 15, or conservative substitutions; e.g., less than 20, 15, 14, 13, 12, 11, 10, 9, 8, 7, 6, 5, 4, 3, or 2 conservative substitutions). In other embodiments, the differences include one or more non-conservative substitutions (e.g., up to 1, 2, 3, 4, 5, 6, 7, 8, 9, 10, 11, 12, 13, 14, 15, or 20 non-conservative substitutions; e.g., less than 20, 15, 14, 13, 12, 11, 10, 9, 8, 7, 6, 5, 4, 3, or 2 non-conservative substitutions). In yet other embodiments, the differences correspond to the amino acid residues found in the corresponding position of the mNC amino acid sequence shown in FIG. 12A. The polypeptide can possess the chitin-binding activity and/or chitinase catalytic activity of SEQ ID NO:2 or 4.

In one embodiment, the pNC protein or polypeptide comprises, or consists of, the amino acid sequence shown in SEQ ID NO:2 or SEQ ID NO:4, or an amino acid sequence substantially identical thereto (e.g., an amino acid sequence about 83%, 84%, 85%, 87%, 90%, 95%, 98% or more identical to the amino acid sequence of SEQ ID NO:2 or SEQ ID NO:4), or a fragment thereof (e.g., a biologically active portion thereof as described herein); or an amino acid sequence encoded by a nucleic acid molecule comprising, or consisting of, the nucleotide sequence of SEQ ID NO:1 or SEQ ID NO:3, or a nucleotide sequence substantially identical thereto (e.g., a nucleotide sequence about 85%, 87%, 90%, 95%, 98% or more identical to the nucleotide sequence shown in SEQ ID NO: 1 or SEQ ID NO:3), or a fragment thereof; or a nucleotide sequence that hybridizes under a stringent hybridization condition described herein to a nucleic acid molecule comprising the nucleotide sequence of SEQ ID NO:1 or SEQ ID NO:3, wherein the nucleic acid encodes a full length pNC protein or an active fragment thereof, e.g., a fragment as described herein. In some embodiments, the polypeptide possesses the chitin-binding activity and/or chitinase catalytic activity of SEQ ID NO:2 or 4. In other embodiments, the pNC polypeptide is a fragment of at least 90, 100, 150, 200, 250, 300, 350, 400 or more contiguous amino acids of SEQ ID NO:2 or SEQ ID NO:4. Exemplary mNC fragments that can be used as an immunogen comprise, or consist of, about amino acids 22-475, 22-408, 22-427, 22-474, 409-427, 428-474, or about amino acids 1-21, 22-120, 22-180, 22-240, 22-300, 22-360, 22-400, 120-150, 150-170, 170-180, 180-190, 190-220, 220-250, 250-270, 270-280, 280-290, 290-300, 300-320, 320-330, 330-340, 340-350, 350-360, 360-390, 390-400, 400-410, 410-420, 420-430, 430-440, 440-450, 450-460, 460-470, or 470-476, of SEQ ID NO:2. The invention also features peptides, e.g., of at least 5 or 6 amino acids from the above sequence. The peptides can be included in a heterologous protein (e.g., a protein other than a pNC), a chimeric protein or can be in an isolated peptide, e.g., one that does not include other sequences.

In a related aspect, the invention provides pNC polypeptides or fragments operatively linked to non-pNC polypeptides to form fusion proteins. The pNC polypeptides or fragments can also be fused or conjugated to other compounds, e.g., a carrier. In some embodiments, the polypeptide or fragment possesses the chitin-binding activity and/or chitinase catalytic activity of SEQ ID NO:2 or 4.

In another aspect, the invention features antibody molecules, e.g., antibodies and antigen-binding fragments thereof, which interact with, or more preferably specifically bind to pNC polypeptides or fragments thereof. In one embodiment, the antibody molecules bind to mNC and other chitinases, e.g., human AMCase and/or human chitotriosidase. In other embodiments, the antibody molecules bind specifically to a pNC polypeptide and does not substantially cross-react with other chitinases (e.g., binds to other chitinases with an affinity that is less than 50%, 40%, 30%, 20%, 10%, 5% or less compared to the affinity of the antibody molecule to the pNC). In one embodiment, the antibody molecule binds to an pNC epitope located on a chitinase catalytic domain (e.g., about amino acids 22 to 408 of SEQ ID NO:2), a linker (e.g., about amino acids 409 to 427 of SEQ ID NO:2), and/or a chitin-binding domain (e.g., about amino acids 428 to 474 of SEQ ID NO:2). In another embodiment, the antibody molecules reduce or inhibit one or more activities of a chitinase, e.g., a pNC polyptide as described herein, and/or one or both of a human AMCase or a human chitotriosidase. For example, the antibody molecule can inhibit or reduce about 20, 50, 60, 70, 80, 85, 90, 95% of one or more activities of a chitinase, e.g., a pNC polyptide as described herein, and/or one or more of a human AMCase or a human chitotriosidase.

In one aspect, the invention features a method of providing an antibody molecule that specifically binds to a primate, e.g., a human, chitinase protein. The method includes: providing a non-human chitinase, e.g., an mNC or fragment thereof (e.g., an antigen that comprises at least a portion of the mNC protein as described herein, the portion being homologous to (at least about 70, 75, 80, 85, 87, 90, 92, 94, 95, 96, 97, or 98% identical to) a corresponding portion of a human chitinase protein, but differing by at least one amino acid (e.g., at least one, two, three, four, five, six, seven, eight, or nine amino acids)); obtaining an antibody molecule that specifically binds to the non-human chitinase or fragment thereof; and evaluating if the antibody molecule specifically binds to the human chitinase protein, or evaluating efficacy of the antibody molecule in modulating, e.g., inhibiting, the activity of the human chitinase protein. The method can further include administering the antibody molecule to a subject, e.g., a human or non-human primate. In one embodiment, the human chitinase protein is a human AMCase or a human chitotriosidase. The non-human protein can be from a non-human primate, e.g., a rhesus monkey, a cynomolgus monkey, or a pigtail macaque. In embodiments, the non-human protein comprises, or consists of, a full-length mature or precursor sequence, or at least a portion of the mNC protein, as described herein. Exemplary mNC fragments that can be used as an immunogen comprise, or consist of, about amino acids 22-475, 22-408, 22-427, 22-474, 409-427, 428-474, or about amino acids 1-21, 22-120, 22-180, 22-240, 22-300, 22-360, 22-400, 120-150, 150-170, 170-180, 180-190, 190-220, 220-250, 250-270, 270-280, 280-290, 290-300, 300-320, 320-330, 330-340, 340-350, 350-360, 360-390, 390-400, 400-410, 410-420, 420-430, 430-440, 440-450, 450-460, 460-470, or 470-476, of SEQ ID NO:2.

In one embodiment, the step of obtaining the antibody molecule comprises using a protein expression library, e.g., a phage or ribosome display library. For example, the library displays antibody molecules such as Fab's or scFv's. In one embodiment, the step of obtaining the antibody molecule comprises immunizing an animal using the non-human chitinase antigen as an immunogen. For example, the animal can be a rodent, e.g., a mouse or rat. The animal can be a transgenic animal that has at least one human immunoglobulin gene.

In another aspect, the invention features a method of screening, or evaluating, the ability of a compound, e.g., a test compound, to interact with a primate chitinase (e.g., a human or non-human primate chitinase). The method includes contacting a pNC polypeptide or nucleic acid (or the expression product thereof), e.g., a mNC or a fragment thereof as described herein, with the test compound; and evaluating a change in one or more activities of the pNC polypeptide or nucleic acid (or the expression product thereof) in the presence of the test compound, relative to a predetermined level, e.g., a control sample without the test compound. In embodiments, the activities evaluated include a change in the expression, binding (e.g., chitinase-binding), or enzymatic activity of the pNC polypeptide or nucleic acid (or the expression product thereof). The contacting step can be effected in vitro (in cultured cells or a reconstituted system) or in vivo (e.g., by administering the test compound to a non-human subject). The contacting step(s) and/or the administration of the test compound can be repeated.

In a related aspect, the invention features a method for identifying a compound, e.g., a test compound, which modulates (e.g., inhibits) the activity of a pNC polypeptide as described herein, or the expression of a pNC nucleic acid (or the activity of the expression product thereof) as described herein. The method includes contacting the pNC polypeptide or nucleic acid (or the expression product thereof) with a test compound; and determining the effect of the test compound on the activity of the polypeptide or nucleic acid (or the expression product thereof) to thereby identify a compound which modulates (e.g., inhibits) the activity of the polypeptide or nucleic acid (or the expression product thereof), e.g., relative to a predetermined level, e.g., a control sample without the test compound. Such agents are useful for treating or preventing a chitinase-associated disorder (e.g., a disorder as described herein). The contacting step can be effected in vitro (in cultured cells or a reconstituted system) or in vivo (e.g., by administering the test compound to a non-human subject). The contacting step(s) and/or the administration of the test compound can be repeated.

In certain embodiments, the test compound is a peptide, a phosphopeptide, a small molecule (e.g., a member of a combinatorial or natural product library), a nucleic acid, an antisense molecule, a ribozyme, an RNAi, a triple helix molecule, an antibody molecule, a chitinase inhibitor or an analogue thereof, or any combination thereof.

In one embodiment, the test compound modulates (e.g., decreases or increases) the activity or expression of a pNC polypeptide or nucleic acid. For example, the expression of a pNC nuclei acid is modulated by e.g., altering mRNA transcription, mRNA stability, etc.

In other embodiments, the test compound is an inhibitor of chitinase activity. Exemplary inhibitors that can be tested include antibody molecules (e.g., antibody molecules that bind to a catalytic or a chitinase-binding domain, and/or reduce the chitinase activity of pNC). Known chitinase inhibitors, or analogues thereof, can also be tested using the methods of the invention. Examples of chitinase inhibitors include allosamidin, glucoallosamindin-A or -B, methyl-N-demethylallosamidin, demethylallosamidin, didemethylallosamidin, styloguanidine, dipeptide cyclo-(L-Arg-D-Pro) or (L-Arg-L-Pro), or (D-Arg-D-Pro), riboflavin, or analogues thereof.

In embodiments where the test compound is an inhibitor of a pNC-activity, the method further includes identifying the test compound as a candidate compound for treating chitinase-associated disorders. Exemplary chitinase-associated disorders include inflammatory disorders (e.g., lung inflammation), respiratory disorders (e.g., asthma, including allergic and non-allergic asthma, chronic obstructive pulmonary disease (COPD), emphysema), as well as conditions involving airway inflammation, eosinophilia, interstitial lung disease, chronic obstructive lung disease, bronchitis, pneumonia, fibrotic disorders (e.g., cystic fibrosis, liver fibrosis, and pulmonary fibrosis); scleroderma; atopic disorders (e.g., atopic dermatitis, urticaria, eczema, allergic rhinitis, and allergic enterogastritis), and inflammatory bowel disease.

The in vitro methods can further include evaluating the test compound in vivo, e.g., in a non-human subject.

In in vivo embodiments, the method includes administering the test compound to a subject, e.g., a non-human animal (e.g., a sheep, a non-human primate (e.g., a cynomolgus monkey), or a rodent), and evaluating a change in one or more of parameters of chitinase-associated disorders. For example, in asthmatic animal models, one or more of the following can be evaluated in the presence or absence of the test compound: (i) detecting a change in the number of inflammatory cells (e.g., eosinophils, macrophages, neutrophils) into the airways; (ii) measuring eotaxin levels; (iii) detecting in basophil histamine release; and/or (iv) detecting IgE titers. A change, e.g., a reduction, in the level of one or more of (i)-(iv) relative to a predetermined level (e.g., comparing before and after treatment) indicates that the test compound is effectively reducing airway eosinophilia in the subjects, and thus is a candidate for treating inflammatory pulmonary conditions (e.g., asthma, COPD and emphysema).

Without wishing to be bound by theory, Applicants have discovered that the mNC disclosed herein has a specific activity about 40-fold higher than human AMCase. The IC₅₀ concentration, i.e., the concentration of the chitinase inhibitor, in this case, allosamidin, required to achieve a half-maximal inhibition of mNC activity in vitro, is about 10-fold higher than the amount of inhibitor required to inhibit human AMCase. In embodiments where the test compound is evaluated in a non-human primate model (e.g., a cynomolgus monkey), the inhibitory concentration (IC₅₀ value) obtained is normalized by a factor of about 1 to 100-fold, 5 to 50-fold, 7 to 20-fold, or 10-fold, prior to administration to a human subject, e.g., prior to administration to a patient undergoing a therapeutic or prophylactic protocol. Typically, the human subject is suffering from, or at risk of having, a chitinase-associated disorder as described herein.

The mNC can have a Kₘ value that is lower than the hAMCase, e.g., approximately 2-, 3-, 4-, 5-, 6-, 7-, 8-, 9-, or 10-fold lower. For example, the mNC can have a Kₘ of about 6 μM; hAMCase can have a Kₘ of about 42 μM.

The mNC can have a k_{cat} value that is higher than the hAMCase, e.g., approximately 2-, 3-, 4-, 5-, 6-, 7-, 8-, 9-, or 10-fold higher. For example, the mNC can have a kK_{cat} of about 32 s⁻¹; hAMCase can have a k_{cat} of about 3 s⁻¹.

In another aspect, the invention features a method of screening, or evaluating, the ability of a compound, e.g., a test compound, to interact with a primate chitinase (e.g., a human or non-human primate chitinase). The method includes administering the test compound to a non-human subject; and evaluating a change in one or more activities of the pNC polypeptide or nucleic acid in the presence of the test compound, relative to a predetermined level, e.g., before or after administration of the test compound. In embodiments, a change in one or more parameters of a chitinase-associated disorder is evaluated. For example, in asthmatic animal models, one or more of the following can be evaluated in the presence or absence of the test compound: (i) detecting a change in the number of inflammatory cells (e.g., eosinophils, macrophages, neutrophils) into the airways; (ii) measuring eotaxin levels; (iii) detecting basophil histamine release; and/or (iv) detecting IgE titers. A change, e.g., a reduction, in the level of one or more of (i)-(iv) relative to a predetermined level (e.g., comparison before and after treatment) indicates that the test compound is effectively reducing airway eosinophilia in the subjects, and thus is a candidate for treating inflammatory pulmonary conditions (e.g., asthma, COPD and emphysema). In embodiments where the test compound is evaluated in a non-human primate model (e.g., a cynomolgus monkey), the inhibitory concentration (IC₅₀ value) obtained is normalized by a factor of about 1 to 100-fold, 5 to 50-fold, 7 to 20-fold, or 10-fold, prior to administration to a human subject, e.g., prior to administration to a patient undergoing a therapeutic or prophylactic protocol. Typically, the human subject is suffering from, or at risk of having, a chitinase-associated disorder as described herein.

In still another aspect, the invention provides a method for modulating (e.g., inhibiting) a pNC polypeptide or nucleic acid expression or activity, e.g. using the screened compounds. In certain embodiments, the methods are effected in vivo and include treatment (e.g., reduction, amelioration, or curing one or more symptoms) of a chitinase-associated disorder or condition in a subject. Exemplary chitinase-associated disorders include inflammatory disorders (e.g., lung inflammation), respiratory disorders (e.g., asthma, including allergic and non-allergic asthma, chronic obstructive pulmonary disease (COPD), emphysema), as well as conditions involving airway inflammation, eosinophilia, interstitial lung disease, chronic obstructive lung disease, bronchitis, pneumonia, fibrotic disorders (e.g., cystic fibrosis, liver fibrosis, and pulmonary fibrosis); scleroderma; atopic disorders (e.g., atopic dermatitis, urticaria, eczema, allergic rhinitis, and allergic enterogastritis), and inflammatory bowel disease.

In embodiments, the subject is a human suffering from, or at risk of having, a chitinase-associated disorder, e.g., a disorder as described herein.

In yet another aspect, the invention features a method of treating or preventing a chitinase-associated disorder (e.g., a disorder as described herein), in a subject. The method includes administering to the subject a chitinase inhibitory agent (e.g., a compound identified using the methods described herein), in an amount effective to treat or prevent the chitinase-associated disorder.

In some aspects, the invention provides the use of a chitinase inhibitory agent (e.g., a compound identified using the methods described herein) for use in therapy.

In some aspects, the invention provides the use of a chitinase inhibitory agent (e.g., a compound identified using the methods described herein) for use in the preparation of a medicament for the treatment or prevention of a chitinase-associated disorder (e.g., a disorder as described herein).

The invention also features a method of diagnosing a chitinase-associated disorder (e.g., a disorder as described herein), in a subject, e.g., a human or non-human primate. The method includes evaluating the expression or activity of a pNC nucleic acid or polypeptide, such that, a difference in the level of pNC nucleic acid or polypeptide relative to a normal subject or a cohort of normal subjects is indicative of the disorder.

In one embodiment, the evaluating step occurs in vitro or ex vivo. For example, a sample, e.g., a blood or sputum sample, is obtained from the subject.

In a preferred embodiment, the evaluating step occurs in vivo. For example, by administering to the subject a detectably labeled agent that interacts with the pNC nucleic acid or polypeptide, such that a signal is generated relative to the level of activity or expression of the pNC nucleic acid or polypeptide.

The invention also provides assays for determining the activity of, or the presence or absence of a pNC polypeptides or nucleic acid molecules in a biological sample, including for disease diagnosis.

In a further aspect, the invention provides assays for determining the presence or absence of a genetic alteration in a pNC polypeptide or nucleic acid molecule (e.g., by nucleic acid or amino acid sequencing or restriction enzyme cleavage patterns), including for disease diagnosis.

In a preferred embodiment, the contacting step occurs in vitro or ex vivo. For example, a sample, e.g., a blood or sputum sample, is obtained from the subject.

In still another aspect, the invention features a method for evaluating the efficacy of a treatment of a chitinase-associated disorder, e.g., a disorder disclosed herein, in a subject, e.g., a human. The method includes treating a subject with a protocol under evaluation; assessing the expression of a pNC nucleic acid or pNC polypeptide, such that a change in the level of the nucleic acid or the polypeptide after treatment, relative to the level before treatment, is indicative of the efficacy of the treatment of the disorder.

In embodiments, the evaluating step occurs in vitro or ex vivo. For example, a sample, e.g., a blood or sputum sample, is obtained from the subject.

In other embodiments, the evaluating step occurs in vivo. For example, by administering to the subject a detectably labeled agent that interacts with the pNC nucleic acid or polypeptide, a signal is generated relative to the level of activity or expression of the pNC nucleic acid or polypeptide.

In another aspect, the invention features a two dimensional array having a plurality of addresses, each address of the plurality being positionally distinguishable from each other address of the plurality, and each address of the plurality having a unique capture probe, e.g., a nucleic acid or peptide sequence. At least one address of the plurality has a capture probe that recognizes a pNC molecule (e.g., a pNC molecule that is labeled with a detectable label). In some embodiments, a secondary agent that is detectably labeled is used to bind to the pNC molecule. In one embodiment, the capture probe is a nucleic acid, e.g., a probe complementary to a pNC nucleic acid sequence. In another embodiment, the capture probe is a polypeptide, e.g., an antibody specific for pNC polypeptides. Also featured is a method of analyzing a sample by contacting the sample to the aforementioned array and detecting binding of the sample to the array.

Other features and advantages of the invention will be apparent from the following detailed description, and from the claims.

### DESCRIPTION OF DRAWINGS

FIG. 1 is a nucleotide sequence of macaque (cynomolgus monkey) novel chitinase (mNC) (SEQ ID NO:1).

FIG. 2 is an amino acid sequence of mNC (SEQ ID NO:2).

FIG. 3A is a nucleotide sequence of the engineered human novel chitinase (hNC) (SEQ ID NO:3).

FIG. 3B shows the nucleotide sequence of the hNC pseudogene sequence with the frameshifting deletion (SEQ ID NO:41).

FIG. 4A is an amino acid sequence of the engineered hNC (SEQ ID NO:4).

FIG. 4B shows the translation of the predicted hNC pseudogene sequence (SEQ ID NOs: 42-47, respectively, in order of appearance).

FIG. 5A is a diagram showing the genetic configuration of human Acidic Mammalian Chitinase (hAMCase) (SEQ ID NO: 29) and EST sequences for TSA-1902L, TSA-1902S, AK098814, and BC036339, which correspond to the indicated hAMCase exon/introns.

FIG. 5B is a diagram showing the genetic configuration of hNC. (*) indicates a stop codon. Exon 5 encodes a catalytic site. Exon 11 encodes a chitin binding domain.

FIGS. 6A-6B are diagrams showing the strategy used to engineer hNC and truncated hNC (mut-hNC), respectively. (A) Black shaded region represents an intron sequence; (1) is SEQ ID NO: 30; (2) is SEQ ID NO: 31; (3) is SEQ ID NO: 32; (4) is SEQ ID NO: 33; (5) is SEQ ID NO: 34; and (6) is SEQ ID NO: 35. (B) Black shaded region represents an intron sequence; (1) is SEQ ID NO: 30; and (2) is SEQ ID NO: 31. (*) indicates a stop codon; (triangle) indicates a deletion.

FIG. 7 is a flow diagram showing the in silico search strategy.

FIGS. 8A-8B show predicted genomic arrangement for (A) human and (B) macaque chitinases gene sequences.

FIG. 9 is a sequence alignment of hAMCase and novel chitinase (NC) exons (EX) 1 and 11 (SEQ ID NOs:37-40, respectively, in order of appearance). Coding regions are shown by underlining; Untranslated regions (UTR) are not underlined. Primers SEQ ID NOs: 5 (sense) and 8 (antisense) are indicated by arrows; matching nucleotides are shown by a star (*).

FIGS. 10A-10D are bar graphs showing human and macaque qRT-PCR data for: (A) transfected constructs expressed in COS cells, including, hAMCase, hNC, and human chitotriosidase (Chitotriosidase); 3 independent experiments are shown for hAMCase and hNC. (B) endogenous hAMCase (human) and mAMCase (monkey) exons 1-3 mRNA expression levels; (C) endogenous hNC (human) and mNC (monkey) exons 1-3 mRNA expression levels; and (D) endogenous hNC (human) and mNC (monkey) exon 8 mRNA expression levels.

FIGS. 11A-11B are bar graphs showing human and macaque qRT-PCR data for; (A) endogenous hAMCase (human) and mAMCase (monkey) exon 11 mRNA expression levels; and (B) endogenous hNC (human) and mNC (monkey) exon 9 mRNA expression levels.

FIGS. 12A-12B are sequence alignments showing (A) hAMCase (SEQ ID NO:29) versus mNC (SEQ ID NO:2); and (B) engineered hNC (SEQ ID NO:4) versus mNC (SEQ ID NO:2). The sequences in (A) revealed an 84% and 82.1% identity at the nucleotide and protein level, respectively. The sequences in (B) revealed a 91.7% and 87.6% identity at the nucleotide and protein level, respectively. Family 18 chitinase catalytic site conserved motif (DGXDXDXE (SEQ ID NO:36), see Detailed Description) is underlined.

FIG. 12C is a diagram showing the predicted genetic configuration of hAMCase, hNC, mAMCase, and mNC. (*) indicates a stop codon. Exon 5 encodes a catalytic site. Exon 11 encodes a chitin binding domain.

FIG. 13 is an illustration showing the strategy used to clone mNC.

FIG. 14A is a bar graph to compare the enzymatic activity of mNC, hNC, and vector (pSMEN2) in conditioned media of transfected COS cells.

FIG. 14B is a line graph showing the enzymatic activity of mNC and hAMCase in the presence of chitobiose-4MU.

FIGS. 15A-15B are bar graphs showing (A) mNC (gray bar) and hAMCase (black bar) specific activity; (B) hAMCase black bar) and mNC (gray bar) and enzymatic activity at pH 2, 3.5, 5, 6.5, and 8.

FIGS. 15C-15D are line graphs showing methylallosamidin inhibition of (C) hAMCase and (D) mNC.

FIG. 16 is a line graph showing that hAMCase (AMCase) or mNC (cyno chitinase) hydrolyze chitin. Chitin RBV was incubated with increasing concentrations of hAMCase (AMCase) (•) or mNC (cyno chitinase) (∘) for 24 hours at 37° C. The signal which signifies the maximal soluble oligosaccharide release by hAMCase (AMCase) was plotted as 100% maximum activity.

### DETAILED DESCRIPTION

The invention is based, at least in part, on the discovery and characterization of two novel primate chitinases and nucleic acids encoding these chitinases, referred to herein collectively as “primate Novel Chitinases” or “pNCs,” or by their individual names “macaque Novel Chitinase” or “mNC,” and “human Novel Chitinase” or “hNC.” mNC was cloned from cynomolgus monkey, and the hNC is an engineered nucleic acid sequence (that encodes a full length protein) based on a human pseudogene truncated at exon 8 of the human chitinase genomic sequence.

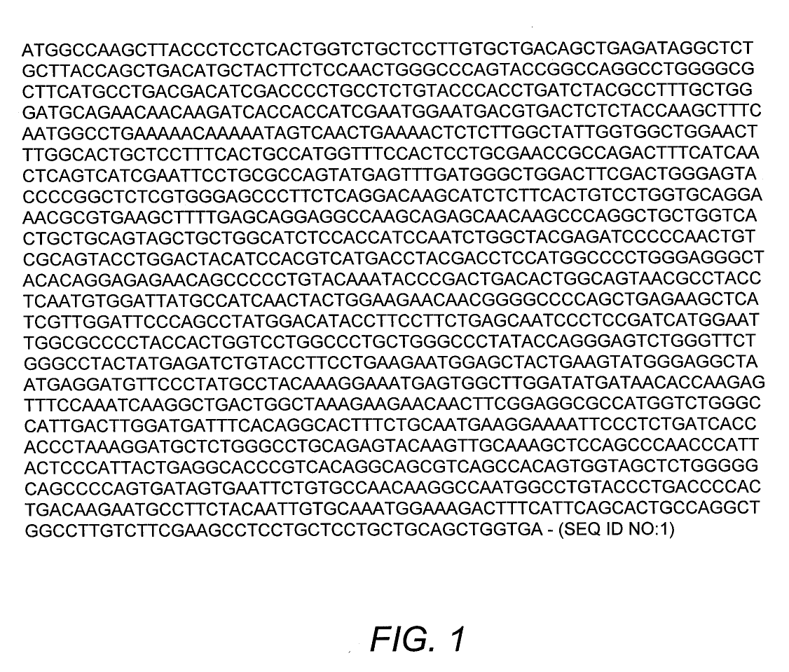

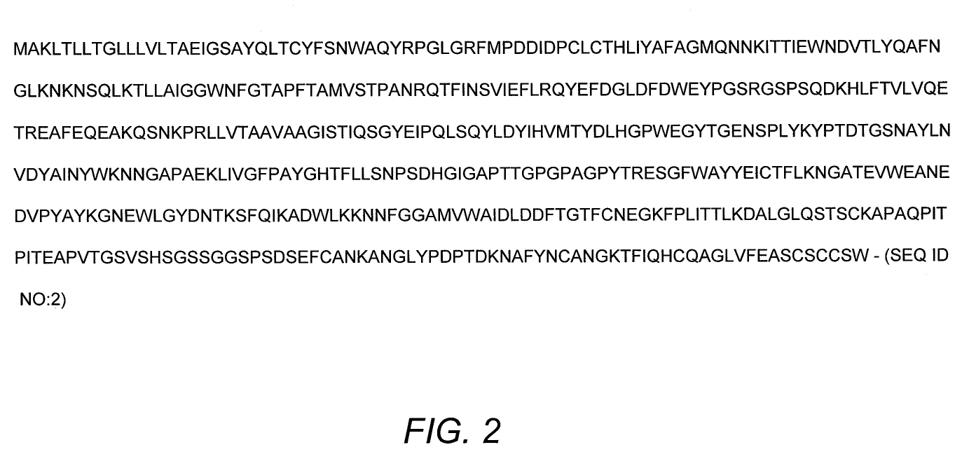

The non-human primate Macaca fascicularis cynomolgus monkey mNC nucleotide sequence (FIG. 1; SEQ ID NO:1), which is approximately 1425 nucleotides long, contains a predicted methionine-initiated coding sequence of about 1425 nucleotides, including the termination codon (5′-TGA-3′). This coding sequence encodes a 485 amino acid protein (FIG. 2; SEQ ID NO:2). This amino acid protein is cleaved at about between amino acids 21 to 22 in SEQ ID NO:2 to yield a mature amino acid protein of about 463 amino acids. mNC has a catalytic domain from about residues 22 to 408, a flexible linker from about amino acids 409 to 427, and a chitin-binding domain from about amino acids 428-474, of SEQ ID NO:2. As described elsewhere in this application, mNC is homologous to the human chitinase AMCase and chitotriosidase, described above.

The mNC gene contains the following regions or other structural features also found in the prototypic AMCase genes (Accession Number AF290004, also disclosed by Boot, R. G. et al. (2001) Journal of Biological Chemistry Vol. 276(9):6770-78) and human chitotriosidase (Accession Number NM_{—}003465; also disclosed by Boot, R. G. et al. (2001) supra), including 11 exons and 10 introns. mNC cDNA (FIG. 1; SEQ ID NO:1) is encoded exons 1 to 11 as follows: exon 1 is between about nucleotides 1 to 25; exon 2 is between about nucleotides 26 to 55; exon 3 is between about nucleotides 56 to 257; exon 4 is between about nucleotides 258 to 314; exon 5 is between about nucleotides 315 to 480; exon 6 is between about nucleotides 481 to 605; exon 7 is between about nucleotides 606 to 729; exon 8 is between about nucleotides 730 to 915; exon 9 is between about nucleotides 916 to 1135; exon 10 is between about nucleotides 1136 to 1177; and exon 11 is between about nucleotides 1178 to 1425.

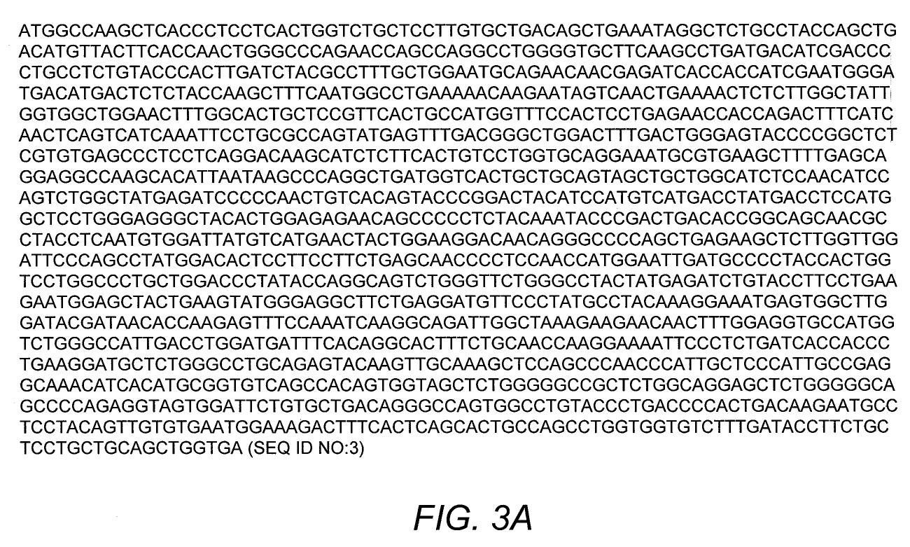

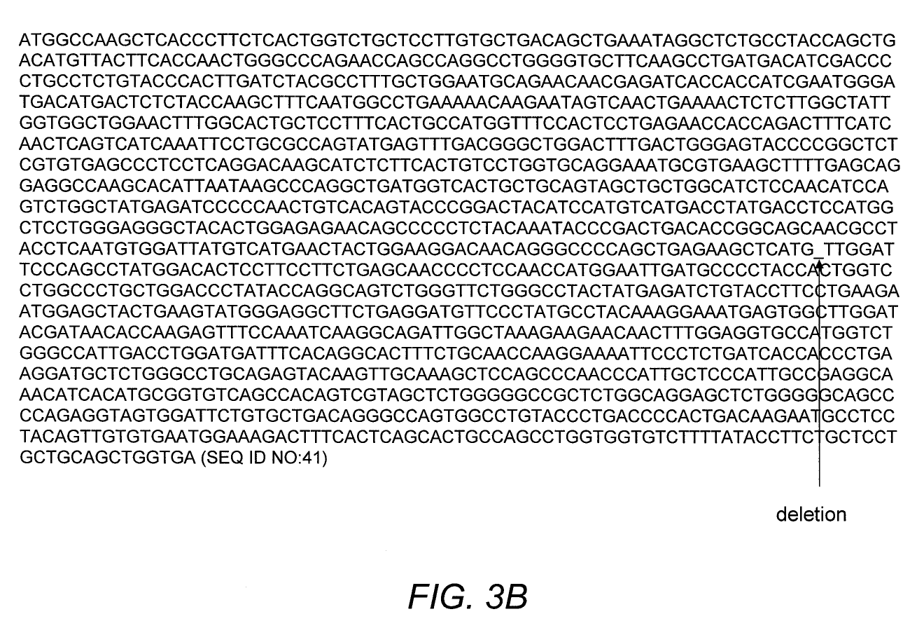

In the majority of humans, the human novel chitinase nucleic acid contains a frameshift deletion that prematurely truncates the encoded protein; the nucleic acid is a pseudogene. This nucleic acid has been engineered to restore the sequence and encode a novel human chitinase. The novel human chitinase (also referred to herein as “hNC” or “engineered hNC”) (FIG. 3A; SEQ ID NO:3), which is approximately 1449 nucleotides long, was engineered to cure a frameshift deletion present in the pseudogene. The novel human chitinase nucleic acid sequence of SEQ ID NO: 3 contains a predicted methionine-initiated coding sequence of about 1449 nucleotides, including the termination codon (5′-TGA-3′). The novel human chitinase gene contains the following regions or other structural features also found in the prototypic AMCase (described above) and human chitotriosidase (described above), including 11 exons and 10 introns. The novel human chitinase gene (FIG. 3A; SEQ ID NO:3) encodes exons 1 to 11 as follows: exon 1 is between about nucleotides 1 to 25; exon 2 is between about nucleotides 26 to 55; exon 3 is between about nucleotides 56 to 257; exon 4 is between about nucleotides 258 to 314; exon 5 is between about nucleotides 315 to 480; exon 6 is between about nucleotides 481 to 605; exon 7 is between about nucleotides 606 to 729; exon 8 is between about nucleotides 730 to 915; exon 9 is between about nucleotides 916 to 1135; exon 10 is between about nucleotides 1136 to 1177; and exon 11 is between about nucleotides 1178 to 1449. The predicted hNC pseudogene sequence is shown in FIG. 3B.

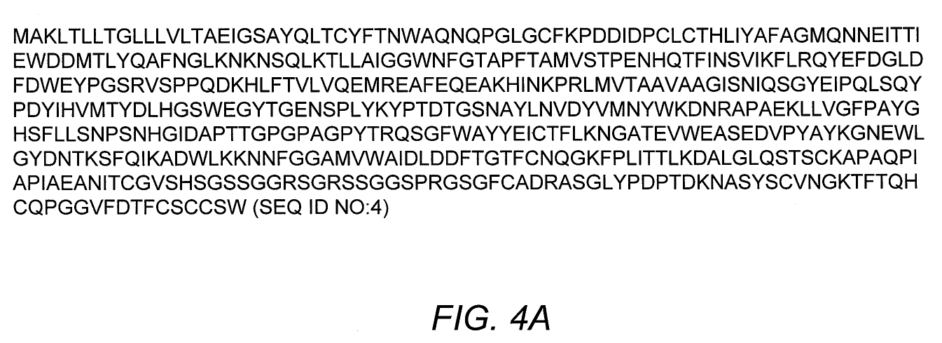

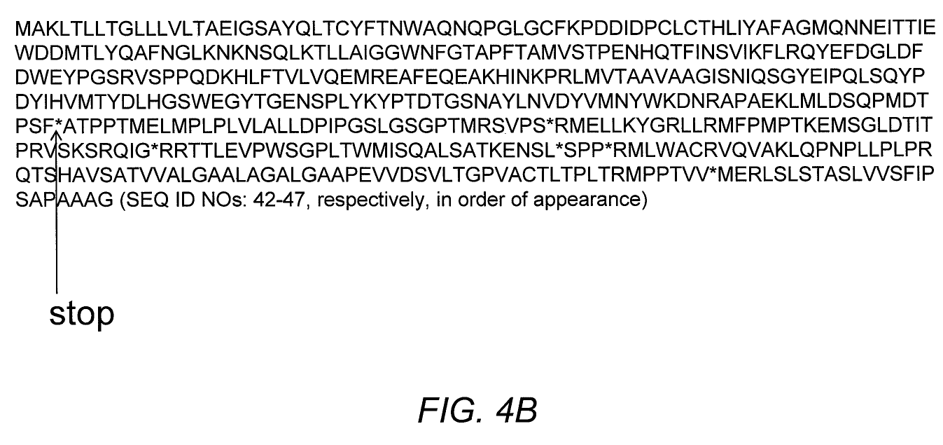

The hNC nucleic acid sequence, engineered to repair a naturally-occurring truncation, encodes a 482 amino acid protein (FIG. 4A; SEQ ID NO:4). This protein contains a secretory leader domain at about amino acid 1 to amino acid 21, a catalytic domain at about amino acid 22 to amino acid 408, a flexible linker domain at about amino acid 409 to amino acid 435, and a chitin-binding domain at about amino acid 436 to 482. The precursor amino acid protein is cleaved at about amino acid 21 in SEQ ID NO:4 to yield a mature amino acid protein of about 461 amino acids. As described elsewhere in this application, novel human chitinase is highly homologous to the human chitinase AMCase, human chitotriosidase, and monkey chitinase. FIG. 4B shows the translation of the hNC pseudogene sequence prior to engineering.

The pNC proteins or polypeptides contain a number of structural characteristics in common with members of the chitinase family, including the catalytic site and chitin binding domain described above. The term “family” when referring to the protein and nucleic acid molecules of the invention means two or more proteins or nucleic acid molecules having a common structural domain or motif and having sufficient amino acid or nucleotide sequence homology as defined herein. Such family members can be naturally or non-naturally occurring and can be from either the same or different species. Members of a family can also have common functional characteristics.

Chitinisases belongs to the glycohydrolase family 18 and are characterized by an eight fold α/β barrel structure. Recent studies have identified chitinases and chitinase-like proteins (CLPs) belonging to the glycohydrolase family 18 in mice and human. The members of the mammalian chitinases and CLPs include chitinase 3-like-1 (CHI3L1), chitotriosidase, YKL-39, Yml, acidic mammalian chitinase (AMCase), oviduct-specific glycoprotein, and stabilin-1-interacting chitinase-like protein (Hakala, et al., J. Biol. Chem., 268:25803-25810, 1993; Boot, et al., J. Biol. Chem., 270:26252-26256, 1995; Hu, et al., J. Biol. Chem., 271:19415-19420, 1996; Jin, et al., Genomics, 54:316-322, 1998; Boot, et al., J. Biol. Chem., 276:6770-6778, 2001; Buhi, W. C., Reproduction, 123:355-362, 2002; Kzhshkowska, et al., Blood, 107:3221-3228, 2006; and Chang, et al., J. Biol. Chem., 276:17497-17506, 2001). Chitotriosidase and AMCase possess chitinase enzymatic activity, whereas other currently identified mammalian chitinases, including CLPs, do not possess this activity (Chang, et al., J. Biol. Chem., 276:17497-17506, 2001). The consensus catalytic site in enzymatically active chitinases is DGXDXDXE (SEQ ID NO:36) on strand β4. Within this motif, the catalytic activity is mediated by glutamic acid (E), which protonates the glycosidic bond with chitin (van Aalten, et al., Proc. Natl. Acad. Sci. USA., 98:8979-8984, 2001).

A pNC polypeptide can include a “chitinase catalytic domain” and/or a “chitin-binding domain,” or regions homologous with a “chitinase catalytic domain” and/or a “chitin-binding domain.”

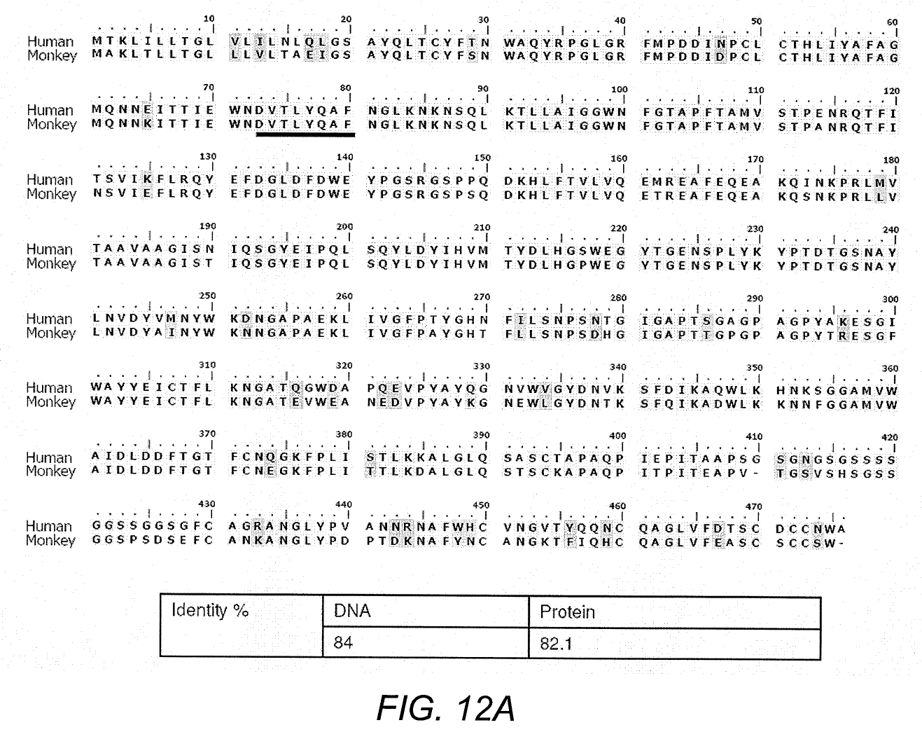

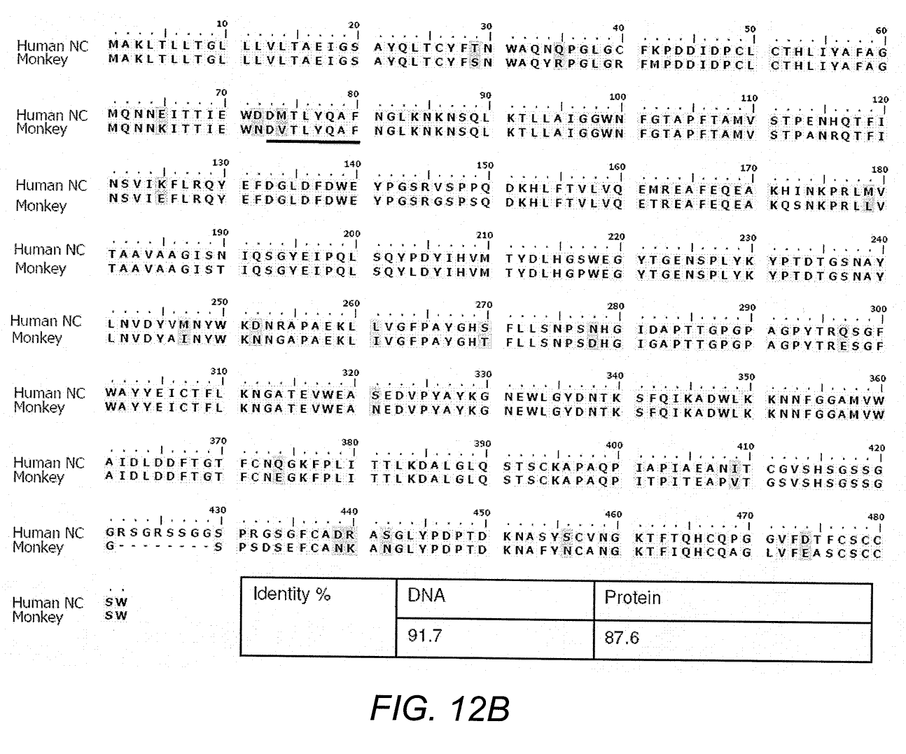

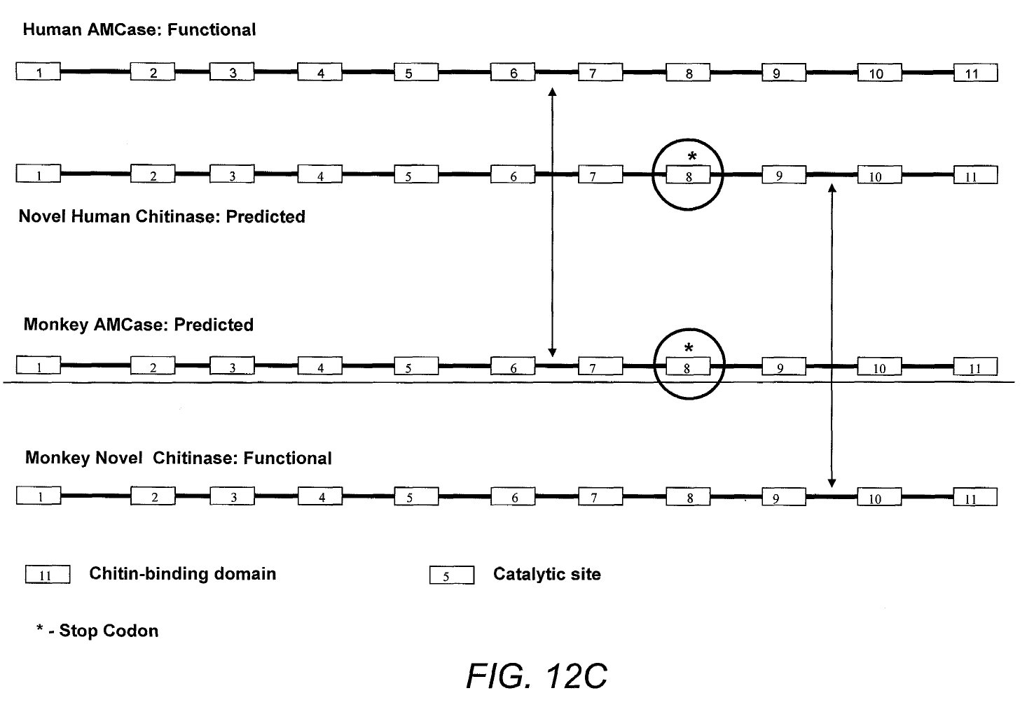

As used herein, the term “chitinase catalytic domain” includes an amino acid sequence about 200 to 400, more typically about 300 to 400 amino acids in length (e.g., from about amino acid residues 22 to 408 of SEQ ID NO:2 or SEQ ID NO:4) and which typically includes a consensus catalytic site having the amino acid sequence DGXDXDXE (SEQ ID NO:36). For example, the consensus catalytic site of mNC is located at amino acids 133 to 138 of SEQ ID NO:2 or SEQ ID NO:4 (FIGS. 12A-12B). In embodiments, pNC polypeptide or protein has a catalytic domain that includes at least about 200 to 400, more typically about 300 to 400 amino acids in length and has at least about 90% 95%, 99%, or 100% homology with amino acid residues 22 to 408 of SEQ ID NO:2 or SEQ ID NO:4.

A pNC protein or polypeptide can further include a “chitin-binding domain.” A “chitin-binding domain” includes an amino acid sequence about 20 to 70, more typically about 40 to 50 amino acids in length and which has at least about 90% 95%, 99%, or 100% homology with amino acid residues 428 to 474 of SEQ ID NO:2 or about 429 to 476 of SEQ ID NO:4.

A pNC protein or polypeptide can also include a linker region connecting the catalytic and chitinase-binding domains. In embodiments, the linker region can have an amino acid sequence about 10 to 30, 15 to 28 amino acid residues in length, and having an amino acid sequence identical or substantially homologous to amino acids 409 to 427 of SEQ ID NO:2, or 409 to 435 of SEQ ID NO:4.

As the pNC polypeptides of the invention may modulate pNC or chitinase-associated activities, they may be useful for developing novel diagnostic and therapeutic agents for chitinase-associated disorders, as described below.

As used herein, a “pNC activity,” “biological activity of pNC” or “functional activity of pNC” refers to an activity exerted by a pNC protein, polypeptide or nucleic acid molecule. For example, a pNC activity can be an activity exerted by pNC in a physiological milieu on, e.g., a pNC-responsive cell or on a pNC substrate, e.g., a chitin-like substrate. A pNC activity can be determined in vivo or in vitro. In one embodiment, a pNC activity is a direct activity, such as an association with a pNC target molecule. A “target molecule” or “binding partner” is a molecule with which a pNC protein binds or interacts in nature. In an exemplary embodiment, pNC is an enzyme for a chitin-like substrate. For example, the pNC proteins of the present invention can have one or more of the following activities or properties: (1) has the ability to bind chitin and/or chitin-like substrates; (2) catalyzes the hydrolysis of chitin and/or chitin-like substrates; (3) has optimal catalytic properties in acidic pH; (4) is inhibited by a chitinase-like inhibitor, such as allosamidin, glucoallosamindin-A or -B, methyl-N-demethylallosamidin, demethylallosamidin, didemethylallosamidin, styloguanidine, dipeptide cyclo-(L-Arg-D-Pro) or (L-Arg-L-Pro), or (D-Arg-D-Pro), riboflavin; (5) has a specific activity at least 10-, 20-, 30-, 40-fold or higher than a human AMCase; (6) is highly expressed in the stomach, and to a lesser extent in the lungs; (7) is upregulated during inflammatory conditions; (8) induces airway hyperresponsiveness in asthmatic patients, particularly patients having allergic asthma.

As used herein, a “biologically active portion” of a pNC protein includes a fragment of a pNC protein which participates in an interaction, e.g., an intramolecular or an inter-molecular interaction. An inter-molecular interaction can be a specific binding interaction or an enzymatic interaction (e.g., the interaction can be transient and a covalent bond is formed or broken). An inter-molecular interaction can be between a pNC molecule and a non-pNC molecule or between a first pNC molecule and a second pNC molecule (e.g., a dimerization interaction). Biologically active portions of a pNC protein include peptides comprising amino acid sequences sufficiently homologous to, or derived from, the amino acid sequence of the pNC protein, e.g., the amino acid sequence shown in SEQ ID NO:2, which include less amino acids than the full length pNC proteins, and exhibit at least one activity of a pNC protein. Typically, biologically active portions comprise a domain or motif with at least one activity of the pNC protein, e.g., chitin binding, or enzymatic degradation of a chitin or chitin-like substrate. A biologically active portion of a pNC protein can be a polypeptide which is, for example, 30, 50, 100, 200, 300 or more amino acids in length. Biologically active portions of a pNC protein can be used as targets for developing agents which modulate a pNC-associated activity or property as defined herein.

Thus, the pNC proteins and nucleic acids can act as novel diagnostic and therapeutic agents for detecting and/or treating chitinase-associated diseases, including, but not limited to, inflammatory disorders (e.g., lung inflammation), respiratory disorders (e.g., asthma, including allergic and non-allergic asthma, chronic obstructive pulmonary disease (COPD), emphysema), as well as conditions involving airway inflammation, eosinophilia, interstitial lung disease, chronic obstructive lung disease, bronchitis, pneumonia, fibrotic disorders (e.g., cystic fibrosis, liver fibrosis, and pulmonary fibrosis); scleroderma; atopic disorders (e.g., atopic dermatitis, urticaria, eczema, allergic rhinitis, and allergic enterogastritis), and inflammatory bowel disease.

Certain terms applicable to the pNC nucleic acids and polypeptides of the invention are collectively defined herein below. Additional terms are defined throughout the application.

The pNC protein, fragments thereof, and derivatives and other variants of the sequence in SEQ ID NO:2 or SEQ ID NO:4 thereof are collectively referred to as “polypeptides or proteins of the invention” or “pNC polypeptides or proteins.” Nucleic acid molecules encoding such polypeptides or proteins are collectively referred to as “nucleic acids of the invention” or “pNC nucleic acids.” pNC molecules refer to pNC nucleic acids, polypeptides and antibodies. The present invention is directed, in part, to pNC polypeptides or nucleic acids comprising, or consisting of, the amino acid or nucleotide sequence specified, or sequences substantially identical or similar thereto, e.g., sequences at least 85%, 90%, 95% identical or higher to the sequence specified.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to pNC nucleic acid (SEQ ID NO: 1) molecules of the invention. BLAST protein searches can be performed with the XBLAST program, score=50, wordlength=3 to obtain amino acid sequences homologous to pNC (SEQ ID NO: 1) protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

Particular pNC polypeptides of the present invention have an amino acid sequence substantially identical to the amino acid sequence of SEQ ID NO:2 or SEQ ID NO:4. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%. 91%, 92%, 93%, 94%, 95%, 96%, 97%, 98% or 99% identity to SEQ ID NO:2 or SEQ ID NO:4 are termed substantially identical.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%. 91%, 92%, 93%, 94%, 95%, 96%, 97%, 98% or 99% identity to SEQ ID NO:1 or 3 are termed substantially identical.

“About” and “approximately” shall generally mean an acceptable degree of error for the quantity measured given the nature or precision of the measurements. Typically, exemplary degrees of error are within 20 percent (%), preferably within 10%, and more preferably within 5% of a given value or range of values. In the context of residues in nucleic acid or amino acid sequences, “about” refers to variation of up to 5 residues (e.g., 5, 4, 3, 2, or 1 residue variation from a disclosed sequence or a particular residue in a disclosed sequence).

Various aspects of the invention are described in further detail below.

### I. Isolated Nucleic Acid Molecules and Fragments

In one aspect, the invention provides an isolated or purified nucleic acid molecule that encodes a pNC polypeptide described herein, e.g., a full-length pNC protein or a fragment thereof, e.g., a biologically active portion of pNC protein. Also included is a nucleic acid fragment suitable for use as a hybridization probe, which can be used, e.g., to identify a nucleic acid molecule encoding a polypeptide of the invention, pNC mRNA, and fragments suitable for use as primers, e.g., PCR primers for the amplification or mutation of nucleic acid molecules.

As used herein, the term “nucleic acid molecule” includes DNA molecules (e.g., a cDNA or genomic DNA), RNA molecules (e.g., an mRNA) and analogs of the DNA or RNA. A DNA or RNA analog can be synthesized from nucleotide analogs. The nucleic acid molecule can be single-stranded or double-stranded, but preferably is double-stranded DNA. As used herein, a “naturally-occurring” nucleic acid molecule refers to an RNA or DNA molecule having a nucleotide sequence that occurs in nature. For example a naturally occurring nucleic acid molecule can encode a natural protein.

As used herein, the terms “gene” and “recombinant gene” refer to nucleic acid molecules which include at least an open reading frame encoding a pNC protein. The gene can optionally further include non-coding sequences, e.g., regulatory sequences and introns. Preferably, a gene encodes a primate pNC protein or derivative thereof.

The term “isolated nucleic acid molecule” or “purified nucleic acid molecule” includes nucleic acid molecules that are separated from other nucleic acid molecules present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

A nucleic acid molecule of the invention can include only a portion of the nucleic acid sequence of SEQ ID NO: 1 or SEQ ID NO:3. For example, such a nucleic acid molecule can include a fragment which can be used as a probe or primer or a fragment encoding a portion of a pNC protein, e.g., an immunogenic or biologically active portion of a pNC protein. A fragment can comprise those nucleotides of SEQ ID NO: 1 or SEQ ID NO:3, which encode a chitinase-binding domain, a chitinase catalytic domain, or a linker, of pNC. The nucleotide sequence determined from the cloning of the pNC gene allows for the generation of probes and primers designed for use in identifying and/or cloning other chitinase family members, or fragments thereof, as well as pNC homologues, or fragments thereof, from other species.

In another embodiment, a nucleic acid includes a nucleotide sequence that includes part, or all, of the coding region and extends into either (or both) the 5′ or 3′ noncoding region. Other embodiments include a fragment which includes a nucleotide sequence encoding an amino acid fragment described herein.

In one embodiment, the isolated nucleic acid molecule encodes a polypeptide comprising, or consisting of, the amino acid sequence of SEQ ID NO:2 or SEQ ID NO:4, or an amino acid sequence substantially identical thereto (e.g., an amino acid sequence about 83%, 84%, 85%, 87%, 90%, 95%, 98% or more identical to the amino acid sequence of SEQ ID NO:2 or SEQ ID NO:4), or a fragment thereof (e.g., a biologically active portion thereof as described herein). In other embodiment, the invention provides isolated pNC nucleic acid molecules comprising, or consisting of, the nucleotide sequence shown in SEQ ID NO:1 or SEQ ID NO:3, or a sequence substantially identical thereto (e.g., a nucleotide sequence about 85%, 87%, 90%, 95%, 98% or more identical to the nucleotide sequence shown in SEQ ID NO:1 or SEQ ID NO:3), or a fragment thereof. In embodiments, the pNC nucleic acid is a fragment of at least about 100, 300, 350, 400, 450, 500, 600, 700, 800, 900, 1000, 1100, 1200 or more contiguous nucleotides of SEQ ID NO:1 or SEQ ID NO:3. In other embodiments, the invention provides a nucleic acid molecule which hybridizes under a stringent hybridization condition described herein to a nucleic acid molecule comprising the nucleotide sequence of SEQ ID NO:1 or SEQ ID NO:3 (or a complement thereof), wherein the nucleic acid encodes a full length pNC protein or an active fragment thereof (e.g., a fragment as described herein).

In another related aspect, the invention provides nucleic acid fragments suitable as primers or hybridization probes for the detection of pNC-encoding nucleic acids. A probe or primer can be derived from the sense or anti-sense strand of a nucleic acid which encodes.

In embodiments, the primers or hybridization probes distinguish between the pNCs disclosed herein, or the sequences disclosed herein and chitinases from other species, e.g., human AMCase or human chitotriosidase. The nucleic acid fragments of the invention can also be used to distinguish between a chitinase nucleotide sequence encoding a functional or a non-functional chitinase, e.g., a truncated or a pseudogene chitinase. Exemplary nucleotide sequences that can be used to detect specifically the pNC-encoding nucleic acids include the following nucleotides: about nucleotides 513-524, 780-784, 830-835, 843-847, 1117-1124, 1211-1237, 1211-1226, 1220-1237, 1262-1288, 1295-1305, 1312-1320, 1360-1376, 1404-1430, 1404-1414 and 1418-1430, of SEQ ID NO:1 or SEQ ID NO: 3, or a complementary nucleotide sequence thereof. Examples of primers or probes that can be used as disclosed herein, e.g., as SEQ ID NOs: 30-35.

In embodiments, the nucleic acid is a probe which is at least 5 or 10, and less than 200, more preferably less than 100, or less than 50, base pairs in length. It should be identical, or differ by 1, or less than in 5 or 10 bases, from a sequence disclosed herein. If alignment is needed for this comparison the sequences should be aligned for maximum homology. “Looped” out sequences from deletions or insertions, or mismatches, are considered differences.

In another embodiment, a set of primers is provided, e.g., primers suitable for use in a PCR, which can be used to amplify a selected region of a pNC sequence, e.g., a catalytic or chitin-binding domain, region, site or other sequence described herein. The primers should be at least 5, 10, or 50 base pairs in length and less than 100, or less than 200, base pairs in length. The primers should be identical, or differs by one base from a sequence disclosed herein or from a naturally occurring variant. For example, primers suitable for amplifying all or a portion of any of the following regions are provided: a chitinase catalytic domain from about amino acid 22 to 408 of SEQ ID NO:2 or SEQ ID NO:4; a chitin-binding domain from about amino acid 428 to 474 of SEQ ID NO:2, or 436 to 482 of SEQ ID NO:4; and/or a linker from about amino acids 409 to 427 of SEQ ID NO:2, or about amino acids 409 to 435 of SEQ ID NO:4. A nucleic acid fragment can encode an epitope bearing region of a polypeptide described herein.

A nucleic acid fragment encoding a “biologically active portion of a pNC polypeptide” can be prepared by isolating a portion of the nucleotide sequence of SEQ ID NO:1, which encodes a polypeptide having a pNC biological activity (e.g., the biological activities of the pNC proteins are described herein), expressing the encoded portion of the pNC protein (e.g., by recombinant expression in vitro) and assessing the activity of the encoded portion of the pNC protein. For example, a nucleic acid fragment encoding a biologically active portion of pNC includes; a pNC catalytic domain and a pNC chitin binding domain, or a pNC catalytic domain in the absence of a pNC chitin binding domain, or a pNC chitin binding domain in the absence of a pNC catalytic domain

### pNC Nucleic Acid Variants

The invention further encompasses nucleic acid molecules that differ from the nucleotide sequence shown in SEQ ID NO: 1 or SEQ ID NO:3. Such differences can be due to degeneracy of the genetic code (and result in a nucleic acid which encodes the same pNC proteins as those encoded by the nucleotide sequence disclosed herein. In another embodiment, an isolated nucleic acid molecule of the invention has a nucleotide sequence encoding a protein having an amino acid sequence which differs, by at least 1, but less than 5, 10, 20, 50, or 100 amino acid residues that shown in SEQ ID NO:2 or SEQ ID NO:4. If alignment is needed for this comparison the sequences should be aligned for maximum homology. “Looped” out sequences from deletions or insertions, or mismatches, are considered differences.

Nucleic acids of the inventor can be chosen for having codons, which are preferred, or non-preferred, for a particular expression system. E.g., the nucleic acid can be one in which at least one codon, at preferably at least 10%, or 20% of the codons has been altered such that the sequence is optimized for expression in E. coli, yeast, human, insect, or CHO cells.

Nucleic acid variants can be naturally occurring, such as allelic variants (same locus), homologs (different locus), and orthologs (different organism) or can be non naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

In a preferred embodiment, the nucleic acid differs from that of SEQ ID NO: 1 or SEQ ID NO:3, e.g., as follows: by at least one but less than 10, 20, 30, or 40 nucleotides; at least one but less than 1%, 5%, 10% or 20% of the nucleotides in the subject nucleic acid. If necessary for this analysis the sequences should be aligned for maximum homology. “Looped” out sequences from deletions or insertions, or mismatches, are considered differences.

Orthologs, homologs, and allelic variants can be identified using methods known in the art. These variants comprise a nucleotide sequence encoding a polypeptide that is 50%, at least about 55%, typically at least about 70-75%, more typically at least about 80-85%, and most typically at least about 90-95% or more identical to the nucleotide sequence shown in SEQ ID NO:2 or a fragment of this sequence. Such nucleic acid molecules can readily be identified as being able to hybridize under a stringency condition described herein, to the nucleotide sequence shown in SEQ ID NO 2 or SEQ ID NO:4, or a fragment of the sequence. Nucleic acid molecules corresponding to orthologs, homologs, and allelic variants of the pNC cDNAs of the invention can further be isolated by mapping to the same chromosome or locus as the pNC gene.

Preferred variants include those that are correlated with pNC enzymatic activity, e.g., enzymatic degradation of a chitin or chitin-like substrate.

Allelic variants of pNC, e.g., mouse, rat, human pNC, include both functional and non-functional proteins. Functional allelic variants are naturally occurring amino acid sequence variants of the pNC protein within a population that maintain the ability to enzymatically degrade chitin or a chitin-like substrate. Functional allelic variants will typically contain only conservative substitution of one or more amino acids of SEQ ID NO:2 or SEQ ID NO:4, or substitution, deletion or insertion of non-critical residues in non-critical regions of the protein. Non-functional allelic variants are naturally-occurring amino acid sequence variants of the pNC, e.g., human pNC, protein within a population that do not possess enzymatic activity. These variants may, however, be capable of binding to chitin or a chitin like substrate, including, for example, monkey AMCase. Non-functional allelic variants will typically contain a non-conservative substitution, a deletion, or insertion, or premature truncation of the amino acid sequence of SEQ ID NO:2 or SEQ ID NO:4, or a substitution, insertion, or deletion in critical residues or critical regions of the protein.

Moreover, nucleic acid molecules encoding other pNC family members and, thus, which have a nucleotide sequence which differs from the pNC sequences of SEQ ID NO:1 or SEQ ID NO:3 are intended to be within the scope of the invention.

### Antisense Nucleic Acid Molecules, RNAi, Ribozymes and Modified pNC Nucleic Acid Molecules

In embodiments, nucleic acid antagonists are used to decrease expression of an endogenous gene encoding pNC. In one embodiment, the nucleic acid antagonist is an siRNA that targets mRNA encoding pNC. Other types of antagonistic nucleic acids can also be used, e.g., a dsRNA, a ribozyme, a triple-helix former, or an antisense nucleic acid. Accordingly, isolated nucleic acid molecules that are nucleic acid inhibitors, e.g., antisense, RNAi, to a pNC-encoding nucleic acid molecule are provided. Exemplary nucleotide sequences that can be used to modify the expression of the pNC-encoding nucleic acids include the following nucleotides: about nucleotides 513-524, 780-784, 830-835, 843-847, 1117-1124, 1211-1237, 1211-1226, 1220-1237, 1262-1288, 1295-1305, 1312-1320, 1360-1376, 1404-1430, 1404-1414 and 1418-1430, of SEQ ID NO:1 or SEQ ID NO: 3, or a complementary nucleotide sequence thereof. Examples of nucleic acid molecules that can be used as disclosed herein, e.g., as SEQ ID NOs: 30-35.

An “antisense” nucleic acid can include a nucleotide sequence which is complementary to a “sense” nucleic acid encoding a protein, e.g., complementary to the coding strand of a double-stranded cDNA molecule or complementary to an mRNA sequence. The antisense nucleic acid can be complementary to an entire pNC coding strand, or to only a portion thereof. In another embodiment, the antisense nucleic acid molecule is antisense to a “noncoding region” of the coding strand of a nucleotide sequence encoding pNC (e.g., the 5′ and 3′ untranslated regions). Anti-sense agents can include, for example, from about 8 to about 80 nucleobases (i.e., from about 8 to about 80 nucleotides), e.g., about 8 to about 50 nucleobases, or about 12 to about 30 nucleobases. Anti-sense compounds include ribozymes, external guide sequence (EGS) oligonucleotides (oligozymes), and other short catalytic RNAs or catalytic oligonucleotides which hybridize to the target nucleic acid and modulate its expression. Anti-sense compounds can include a stretch of at least eight consecutive nucleobases that are complementary to a sequence in the target gene. An oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

Hybridization of antisense oligonucleotides with mRNA can interfere with one or more of the normal functions of mRNA. The functions of mRNA to be interfered with include all key functions such as, for example, translocation of the RNA to the site of protein translation, translation of protein from the RNA, splicing of the RNA to yield one or more mRNA species, and catalytic activity which may be engaged in by the RNA. Binding of specific protein(s) to the RNA may also be interfered with by antisense oligonucleotide hybridization to the RNA.

Exemplary antisense compounds include DNA or RNA sequences that specifically hybridize to the target nucleic acid, e.g., the mRNA encoding pNC. The complementary region can extend for between about 8 to about 80 nucleobases. The compounds can include one or more modified nucleobases. Modified nucleobases may include, e.g., 5-substituted pyrimidines such as 5-iodouracil, 5-iodocytosine, and C5-propynyl pyrimidines such as C5-propynylcytosine and C5-propynyluracil. Other suitable modified nucleobases include N⁴—(C₁-C₁₂) alkylaminocytosines and N⁴,N⁴—(C₁-C₁₂) dialkylaminocytosines. Modified nucleobases may also include 7-substituted-8-aza-7-deazapurines and 7-substituted-7-deazapurines such as, for example, 7-iodo-7-deazapurines, 7-cyano-7-deazapurines, 7-aminocarbonyl-7-deazapurines. Examples of these include 6-amino-7-iodo-7-deazapurines, 6-amino-7-cyano-7-deazapurines, 6-amino-7-aminocarbonyl-7-deazapurines, 2-amino-6-hydroxy-7-iodo-7-deazapurines, 2-amino-6-hydroxy-7-cyano-7-deazapurines, and 2-amino-6-hydroxy-7-aminocarbonyl-7-deazapurines. Furthermore, N⁶—(C₁-C₁₂) alkylaminopurines and N⁶,N⁶—(C₁-C₁₂) dialkylaminopurines, including N⁶-methylaminoadenine and N⁶,N⁶-dimethylaminoadenine, are also suitable modified nucleobases. Similarly, other 6-substituted purines including, for example, 6-thioguanine may constitute appropriate modified nucleobases. Other suitable nucleobases include 2-thiouracil, 8-bromoadenine, 8-bromoguanine, 2-fluoroadenine, and 2-fluoroguanine. Derivatives of any of the aforementioned modified nucleobases are also appropriate. Substituents of any of the preceding compounds may include C₁-C₃₀ alkyl, C₂-C₃₀ alkenyl, C₂-C₃₀ alkynyl, aryl, aralkyl, heteroaryl, halo, amino, amido, nitro, thio, sulfonyl, carboxyl, alkoxy, alkylcarbonyl, alkoxycarbonyl, and the like. Descriptions of other types of nucleic acid agents are also available. See, e.g., U.S. Pat. Nos. 4,987,071; 5,116,742; and 5,093,246; Woolf et al. (1992) Proc Natl Acad Sci USA; Antisense RNA and DNA, D. A. Melton, Ed., Cold Spring Harbor Laboratory, Cold Spring Harbor, N.Y. (1988); 89:7305-9; Haselhoff and Gerlach (1988) Nature 334:585-59; Helene, C. (1991) Anticancer Drug Des. 6:569-84; Helene (1992) Ann. N.Y. Acad. Sci. 660:27-36; and Maher (1992) Bioassays 14:807-15.

The antisense nucleic acid molecules of the invention are typically administered to a subject (e.g., by direct injection at a tissue site), or generated in situ such that they hybridize with or bind to cellular mRNA and/or genomic DNA encoding a pNC protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

siRNAs are small double stranded RNAs (dsRNAs) that optionally include overhangs. For example, the duplex region of an siRNA is about 18 to 25 nucleotides in length, e.g., about 19, 20, 21, 22, 23, or 24 nucleotides in length. Typically, the siRNA sequences are exactly complementary to the target mRNA. dsRNAs and siRNAs in particular can be used to silence gene expression in mammalian cells (e.g., human cells). siRNAs also include short hairpin RNAs (shRNAs) with 29-base-pair stems and 2-nucleotide 3′ overhangs. See, e.g., Clemens et al. (2000) Proc. Natl. Acad. Sci. USA 97:6499-6503; Billy et al. (2001) Proc. Natl. Sci. USA 98:14428-14433; Elbashir et al. (2001) Nature. 411:494-8; Yang et al. (2002) Proc. Natl. Acad. Sci. USA 99:9942-9947; Siolas et al. (2005), Nat. Biotechnol. 23(2):227-31; 20040086884; U.S. 20030166282; 20030143204; 20040038278; and 20030224432.

In still another embodiment, an antisense nucleic acid of the invention is a ribozyme. A ribozyme having specificity for a pNC-encoding nucleic acid can include one or more sequences complementary to the nucleotide sequence of a pNC cDNA disclosed herein (i.e., SEQ ID NO: 1 or SEQ ID NO:3), and a sequence having known catalytic sequence responsible for mRNA cleavage (see U.S. Pat. No. 5,093,246 or Haselhoff and Gerlach (1988) Nature 334:585-591). For example, a derivative of a Tetrahymena L-19 IVS RNA can be constructed in which the nucleotide sequence of the active site is complementary to the nucleotide sequence to be cleaved in a pNC-encoding mRNA. See, e.g., Cech et al. U.S. Pat. No. 4,987,071; and Cech et al. U.S. Pat. No. 5,116,742. Alternatively, pNC mRNA can be used to select a catalytic RNA having a specific ribonuclease activity from a pool of RNA molecules. See, e.g., Bartel, D. and Szostak, J. W. (1993) Science 261:1411-1418.

pNC gene expression can be inhibited by targeting nucleotide sequences complementary to the regulatory region of the pNC (e.g., the pNC promoter and/or enhancers) to form triple helical structures that prevent transcription of the pNC gene in target cells. See generally, Helene, C. (1991) Anticancer Drug Des. 6:569-84; Helene, C. i (1992) Ann. N.Y. Acad. Sci. 660:27-36; and Maher, L. J. (1992) Bioassays 14:807-15. The potential sequences that can be targeted for triple helix formation can be increased by creating a so-called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

The invention also provides detectably labeled oligonucleotide primer and probe molecules. Typically, such labels are chemiluminescent, fluorescent, radioactive, or colorimetric.

A pNC nucleic acid molecule can be modified at the base moiety, sugar moiety or phosphate backbone to improve, e.g., the stability, hybridization, or solubility of the molecule. For non-limiting examples of synthetic oligonucleotides with modifications see Toulmé (2001) Nature Biotech. 19:17 and Faria et al. (2001) Nature Biotech. 19:40-44. Such phosphoramidite oligonucleotides can be effective antisense agents.

For example, the deoxyribose phosphate backbone of the nucleic acid molecules can be modified to generate peptide nucleic acids (see Hyrup B. et al. (1996) Bioorganic & Medicinal Chemistry 4: 5-23). As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup B. et al. (1996) supra and Perry-O'Keefe et al. Proc. Natl. Acad. Sci. 93: 14670-675.

PNAs of pNC nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of pNC nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. et al. (1996) supra)); or as probes or primers for DNA sequencing or hybridization (Hyrup B. et al. (1996) supra; Perry-O'Keefe supra).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; WO88/09810) or the blood-brain barrier (see, e.g., WO 89/10134). In addition, oligonucleotides can be modified with hybridization-triggered cleavage agents (see, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

The invention also includes molecular beacon oligonucleotide primer and probe molecules having at least one region which is complementary to a pNC nucleic acid of the invention, two complementary regions one having a fluorophore and one a quencher such that the molecular beacon is useful for quantitating the presence of the pNC nucleic acid of the invention in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. No. 5,876,930.

### II. Isolated pNC Polypeptides

In another aspect, the invention features an isolated pNC protein or polypeptides, or fragment, e.g., a biologically active portion, for use as immunogens or antigens to raise or test (or more generally to bind) anti-pNC antibodies. The terms “proteins” and “polypeptides” are used interchangeably herein. pNC protein can be isolated from cells or tissue sources using standard protein purification techniques. pNC protein or fragments thereof can be produced by recombinant DNA techniques or synthesized chemically. Polypeptides of the invention include those which arise as a result of the existence of multiple genes, alternative transcription events, alternative RNA splicing events, and alternative translational and post-translational events. The polypeptide can be expressed in systems, e.g., cultured cells, which result in substantially the same post-translational modifications present when expressed the polypeptide is expressed in a native cell, or in systems which result in the alteration or omission of post-translational modifications, e.g., glycosylation or cleavage, present when expressed in a native cell.

Accordingly, the invention provides an isolated or purified pNC protein or polypeptide, e.g., a biologically active or antigenic fragment thereof (e.g., a mNC portion comprising, or consisting of, a chitinase catalytic domain (e.g., about amino acids 22 to 408 of SEQ ID NO:2), a linker (e.g., about amino acids 409 to 427 of SEQ ID NO:2), and/or a chitin-binding domain (e.g., about amino acids 428 to 474 of SEQ ID NO:2); or e.g., a hNC portion comprising, or consisting of, a chitinase catalytic domain (e.g., about amino acid 22 to 408 of SEQ ID NO:4), a linker (e.g., from about amino acids 409 to 435 of SEQ ID NO:4), and/or a chitin-binding domain (e.g., from about amino acid 436 to 482 of SEQ ID NO:4)). The, pNC can be a mature pNC protein or an unprocessed full length pNC protein (including the signal sequence). In one embodiment, the pNC polypeptide or fragment thereof includes at least one, two, three, five, ten, twenty amino acid residue that differs from human AMCase or human chitotriosidase. Also featured are related pNC polypeptides that differ from human AMCase at one or more of amino acid residues 114, 149, 162, 173, 190, 217, 246, 266, 270, 279, 288, 295, 300, 317, 321, 332, 339, 343, 347, 351, 353, 354, 385, 392, 395, 402, 406, 409, 410, 414, 416, 418, 424, 425, 426, 428, 432, 440, 441, 442, 451, 454, 457, 468, or 471 of the human AMCase sequence shown in FIG. 12A (SEQ ID NO:29). In certain embodiments the differences are conservative substitutions. In other embodiments, the differences are non-conservative substitutions. In yet other embodiments, the differences correspond to the amino acid residues found in the corresponding position of the mNC amino acid sequence shown in FIG. 12A.

The invention also provides an isolated or purified fragment, e.g., a biologically active or antigenic fragment thereof, of any one of SEQ ID NOs:42-47.

In one embodiment, the pNC protein or polypeptide comprises, or consists of, the amino acid sequence shown in SEQ ID NO:2 or SEQ ID NO:4, or an amino acid sequence substantially identical thereto (e.g., an amino acid sequence about 83%, 84%, 85%, 87%, 90%, 95%, 98% or more identical to the amino acid sequence of SEQ ID NO:2 or SEQ ID NO:4), or a fragment thereof (e.g., a biologically active portion thereof as described herein); or an amino acid sequence encoded by a nucleic acid molecule comprising, or consisting of, the nucleotide sequence of SEQ ID NO:1 or SEQ ID NO:3, or a nucleotide sequence substantially identical thereto (e.g., a nucleotide sequence about 85%, 87%, 90%, 95%, 98% or more identical to the nucleotide sequence shown in SEQ ID NO:1 or SEQ ID NO:3), or a fragment thereof; or a nucleotide sequence that hybridizes under a stringent hybridization condition described herein to a nucleic acid molecule comprising the nucleotide sequence of SEQ ID NO:1 or SEQ ID NO:3, wherein the nucleic acid encodes a full length pNC protein or an active fragment thereof, e.g., a fragment as described herein. In other embodiments, the pNC polypeptide is a fragment of at least 90, 100, 150, 200, 250, 300, 350, 400 or more contiguous amino acids of SEQ ID NO:2 or SEQ ID NO:4. Exemplary mNC fragments that can be used as an immunogen comprise, or consist of, about amino acids 22-475, 22-408, 22-427, 22-474, 409-427, 428-474, or about amino acids 1-21, 22-120, 22-180, 22-240, 22-300, 22-360, 22-400, 120-150, 150-170, 170-180, 180-190, 190-220, 220-250, 250-270, 270-280, 280-290, 290-300, 300-320, 320-330, 330-340, 340-350, 350-360, 360-390, 390-400, 400-410, 410-420, 420-430, 430-440, 440-450, 450-460, 460-470, or 470-476, of SEQ ID NO:2. The invention also features peptides, e.g., of at least 5 or 6 amino acids from the above sequence. The peptides can be included in a heterologous protein (e.g., a protein other than a pNC), a chimeric protein or can be in an isolated peptide, e.g., one that does not include other sequences.

An “isolated” or “purified” polypeptide or protein is substantially free of cellular material or other contaminating proteins from the cell or tissue source from which the protein is derived, or substantially free from chemical precursors or other chemicals when chemically synthesized. “Substantially free” means that a preparation of pNC protein is at least 10% pure. In a preferred embodiment, the preparation of pNC protein has less than about 30%, 20%, 10% and more preferably 5% (by dry weight), of non-pNC protein (also referred to herein as a “contaminating protein”), or of chemical precursors or non-pNC chemicals. When the pNC protein or biologically active portion thereof is recombinantly produced, it is also preferably substantially free of culture medium, i.e., culture medium represents less than about 20%, more preferably less than about 10%, and most preferably less than about 5% of the volume of the protein preparation. The invention includes isolated or purified preparations of at least 0.01, 0.1, 1.0, and 10 milligrams in dry weight.

A “non-essential” amino acid residue is a residue that can be altered from the wild-type sequence of pNC without abolishing or substantially altering a pNC activity. Preferably the alteration does not substantially alter the pNC activity, e.g., the activity is at least 20%, 40%, 60%, 70% or 80% of wild-type. An “essential” amino acid residue is a residue that, when altered from the wild-type sequence of pNC, results in abolishing a pNC activity such that less than 20% of the wild-type activity is present. For example, conserved amino acid residues in pNC are predicted to be particularly unamenable to alteration.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a pNC protein is preferably replaced with another amino acid residue from the same side chain family. Alternatively, in another embodiment, mutations can be introduced randomly along all or part of a pNC coding sequence, such as by saturation mutagenesis, and the resultant mutants can be screened for pNC biological activity to identify mutants that retain activity. Following mutagenesis of SEQ ID NO:1 or 3, the encoded protein can be expressed recombinantly and the activity of the protein can be determined.

In embodiments, a pNC polypeptide has one or more of the following characteristics or activities:

(i) it has a molecular weight, e.g., a deduced molecular weight, preferably ignoring any contribution of post translational modifications, amino acid composition or other physical characteristic of SEQ ID NO:2;

(ii) it has an overall sequence similarity of at least 80, 85% 90, or 95%, with a polypeptide a of SEQ ID NO:2;

(iii) it can be found in cynomolgus monkeys;

(iv) it has the ability to bind chitin-like substrates;

(v) it catalyzes the hydrolysis of chitin-like substrates;

(vi) it has optimal catalytic properties in acidic pH;

(vii) it can be inhibited by a chitinase-like inhibitor, such as allosamidin, glucoallosamindin-A or -B, methyl-N-demethylallosamidin, demethylallosamidin, didemethylallosamidin, styloguanidine, dipeptide cyclo-(L-Arg-D-Pro) or (L-Arg-L-Pro), or (D-Arg-D-Pro), riboflavin;

(viii) it has a specific activity at least 10-, 20-, 30-, 40-fold or higher than a human AMCase;

(ix) it can be highly expressed in the stomach, and to a lesser extent in the lungs;

(x) its expression can be upregulated during inflammatory conditions;

(xi) it can induce airway hyperresponsiveness in asthmatic patients, particularly patients having allergic asthma.

In embodiments, the pNC protein, or fragment thereof, differs from the corresponding sequence in SEQ ID NO:2. In one embodiment it differs by at least one but by less than 15, 10 or 5 amino acid residues. In another it differs from the corresponding sequence in SEQ ID NO:2 by at least one residue but less than 20%, 15%, 10% or 5% of the residues in it differ from the corresponding sequence in SEQ ID NO:2. (If this comparison requires alignment the sequences should be aligned for maximum homology. “Looped” out sequences from deletions or insertions, or mismatches, are considered differences.) The differences are, preferably, differences or changes at a non essential residue or a conservative substitution. In embodiments, the differences are not in the catalytic domain.

In embodiments, a pNC polypeptide has one or more of the following characteristics or activities:

(i) it has a molecular weight, e.g., a deduced molecular weight, preferably ignoring any contribution of post translational modifications, amino acid composition or other physical characteristic of SEQ ID NO:4;

(ii) it has an overall sequence similarity of at least 80, 85% 90, or 95%, with a polypeptide a of SEQ ID NO:4;

(iii) it can be found in humans;

(iv) it has the ability to bind chitin-like substrates;

(v) it catalyzes the hydrolysis of chitin-like substrates;

(vi) it has optimal catalytic properties in acidic pH;

(vii) it can be inhibited by a chitinase-like inhibitor, such as allosamidin, glucoallosamindin-A or -B, methyl-N-demethylallosamidin, demethylallosamidin, didemethylallosamidin, styloguanidine, dipeptide cyclo-(L-Arg-D-Pro) or (L-Arg-L-Pro), or (D-Arg-D-Pro), riboflavin;

(viii) it has a specific activity at least 10-, 20-, 30-, 40-fold or lower than a cynomolgus monkeys mNC;

(ix) it can be highly expressed in the stomach, and to a lesser extent in the lungs;

(x) its expression can be upregulated during inflammatory conditions;

(xi) it can induce airway hyperresponsiveness in asthmatic patients, particularly patients having allergic asthma.

In embodiments, the pNC protein, or fragment thereof, differs from the corresponding sequence in SEQ ID NO:4. In one embodiment it differs by at least one but by less than 15, 10 or 5 amino acid residues. In another it differs from the corresponding sequence in SEQ ID NO:4 by at least one residue but less than 20%, 15%, 10% or 5% of the residues in it differ from the corresponding sequence in SEQ ID NO:4. (If this comparison requires alignment the sequences should be aligned for maximum homology. “Looped” out sequences from deletions or insertions, or mismatches, are considered differences.) The differences are, preferably, differences or changes at a non essential residue or a conservative substitution. In embodiments, the differences are not in the catalytic domain.

Other embodiments include a protein that contains one or more changes in amino acid sequence, e.g., a change in an amino acid residue which is not essential for activity. Such pNC proteins differ in amino acid sequence from SEQ ID NO:2 or 4, yet retain biological activity.

In one embodiment, a biologically active portion of a pNC protein includes a chitinase domain. Moreover, other biologically active portions, in which other regions of the protein are deleted, can be prepared by recombinant techniques and evaluated for one or more of the functional activities of a native pNC protein.

### pNC Chimeric or Fusion Proteins

In another aspect, the invention provides pNC chimeric or fusion proteins. As used herein, a pNC “chimeric protein” or “fusion protein” includes a pNC polypeptide linked to a non-pNC polypeptide. A “non-pNC polypeptide” refers to a polypeptide having an amino acid sequence corresponding to a protein which is not substantially homologous to the pNC protein, e.g., a protein which is different from the pNC protein and which is derived from the same or a different organism. The pNC polypeptide of the fusion protein can correspond to all or a portion e.g., a fragment described herein of a pNC amino acid sequence. In a preferred embodiment, a pNC fusion protein includes at least one (or two) biologically active portion of a pNC protein. The non-pNC polypeptide can be fused to the N-terminus or C-terminus of the pNC polypeptide.

The fusion protein can include a moiety which has a high affinity for a ligand. For example, the fusion protein can be a GST-pNC fusion protein in which the pNC sequences are fused to the C-terminus of the GST sequences. Such fusion proteins can facilitate the purification of recombinant pNC. Alternatively, the fusion protein can be a pNC protein containing a heterologous signal sequence at its N-terminus. In certain host cells (e.g., mammalian host cells), expression and/or secretion of pNC can be increased through use of a heterologous signal sequence.

Fusion proteins can include all or a part of a serum protein, e.g., an IgG constant region, or human serum albumin.

The pNC fusion proteins of the invention can be incorporated into pharmaceutical compositions and administered to a subject in vivo. The pNC fusion proteins can be used to affect the bioavailability of a pNC substrate. pNC fusion proteins may be useful therapeutically for the treatment of disorders caused by, for example, (i) aberrant modification or mutation of a gene encoding a pNC protein; (ii) mis-regulation of the pNC gene; and (iii) aberrant post-translational modification of a pNC protein.

Moreover, the pNC-fusion proteins of the invention can be used as immunogens to produce anti-pNC antibodies in a subject, to purify pNC ligands and in screening assays to identify molecules which inhibit the interaction of pNC with a pNC substrate.

Expression vectors are commercially available that already encode a fusion moiety (e.g., a GST polypeptide). A pNC-encoding nucleic acid can be cloned into such an expression vector such that the fusion moiety is linked in-frame to the pNC protein.

### Variants of pNC Proteins

In another aspect, the invention also features a variant of a pNC polypeptide, e.g., which functions as an agonist (mimetics) or as an antagonist. Variants of the pNC proteins can be generated by mutagenesis, e.g., discrete point mutation, the insertion or deletion of sequences or the truncation of a pNC protein. An agonist of the pNC proteins can retain substantially the same, or a subset, of the biological activities of the naturally occurring form of a pNC protein. An antagonist of a pNC protein can inhibit one or more of the activities of the naturally occurring form of the pNC protein by, for example, competitively modulating a pNC-mediated activity of a pNC protein. Thus, specific biological effects can be elicited by treatment with a variant of limited function. Preferably, treatment of a subject with a variant having a subset of the biological activities of the naturally occurring form of the protein has fewer side effects in a subject relative to treatment with the naturally occurring form of the pNC protein.

Variants of a pNC protein can be identified by screening combinatorial libraries of mutants, e.g., truncation mutants, of a pNC protein for agonist or antagonist activity.

Libraries of fragments e.g., N terminal, C terminal, or internal fragments, of a pNC protein coding sequence can be used to generate a variegated population of fragments for screening and subsequent selection of variants of a pNC protein. Variants in which a cysteine residues is added or deleted or in which a residue which is glycosylated is added or deleted are particularly preferred.

Methods for screening gene products of combinatorial libraries made by point mutations or truncation, and for screening cDNA libraries for gene products having a selected property are known in the art. Such methods are adaptable for rapid screening of the gene libraries generated by combinatorial mutagenesis of pNC proteins. Recursive ensemble mutagenesis (REM), a new technique which enhances the frequency of functional mutants in the libraries, can be used in combination with the screening assays to identify pNC variants (Arkin and Yourvan (1992) Proc. Natl. Acad. Sci. USA 89:7811-7815; Delgrave et al. (1993) Protein Engineering 6:327-331).

Cell based assays can be exploited to analyze a variegated pNC library. For example, a library of expression vectors can be transfected into a cell line, e.g., a cell line, which ordinarily responds to pNC in a substrate-dependent manner. The transfected cells are then contacted with pNC and the effect of the expression of the mutant on signaling by the pNC substrate can be detected, e.g., by measuring the enzymatic degradation of a chitin or chitin-like substrate. Plasmid DNA can then be recovered from the cells which score for inhibition, or alternatively, potentiation of signaling by the pNC substrate, and the individual clones further characterized.

A wide variety of assays may be utilized to determine whether the test agent inhibits the activity of the pNC. For example, the amount of reactants remaining and/or products formed in reactions catalyzed by pNC may be quantified. A nonlimiting example of such a reaction is the conversion of chitin or a chitin-like compound to N-acetyl-D-glucosamine. Other reactions include, without limitation, the release of 4-methylumbelliferyl from 4-methylumbelliferyl-tri-N-acetyl chitotrioxide, 4-methylumbelliferyl-D-N,N′-diacetylchitobiose or 4-methylumbelliferyl-D-N,N′,N″-triacetylchitotriose; or the release of p-nitrophenyl from p-nitrophenyl P-D-0-D-N,N′-diacetylchitobiose or p-nitrophenyl-D-N,N′,N″-triacetylchitotriose. To this end, the amount of chitin remaining after contacting pNC with the test agent as a function of time may be determined. Similarly, the amount of N-acetyl-D-glucosamine or 4-methylumbelliferyl or p-nitrophenyl produced after contacting chitinase with the test agent in the presence of, for example, chitin as a function of time may be determined. Various assays may be used to determine the quantity of these products and/or reactants. For example, colorimetric assays may be utilized to determine the quantity of N-acetyl-D-glucosamine as described in, for example, Reissig, J. L., J. Biol. Chers. 217:959-966, 1955. Alternatively, the amount of glucosamine may be determined by chromatographic methods known to the skilled artisan, including high performance liquid chromatography, as described in, for example, Ekblad, A., Plant and Soil, 178:29-35, 1996. Fluorometric assays may be utilized to determine the quantity of 4-methylumbelliferylorp-nitrophenyl as described in, for example, U.S. Pat. No. 5,561,051 (Silverman); Houston, D. R., et al., PNAS, 99: 9127-9232, 2002; Hollak, C. E. M., et al., J. Clin. Invest. 93:1288-1292, 1994; and Hu et al., J. Biol. Chem., 27:19415-194520.

In another aspect, the invention features a method of making a pNC polypeptide, e.g., a peptide having a non-wild type activity, e.g., an antagonist, agonist, or super agonist of a naturally occurring pNC polypeptide, e.g., a naturally occurring pNC polypeptide. The method includes: altering the sequence of a pNC polypeptide, e.g., altering the sequence, e.g., by substitution or deletion of one or more residues of a non-conserved region, a domain or residue disclosed herein, and testing the altered polypeptide for the desired activity.

In another aspect, the invention features a method of making a fragment or analog of a pNC polypeptide a biological activity of a naturally occurring pNC polypeptide. The method includes: altering the sequence, e.g., by substitution or deletion of one or more residues, of a pNC polypeptide, e.g., altering the sequence of a non-conserved region, or a domain or residue described herein, and testing the altered polypeptide for the desired activity. The AMCase may be isolated and purified by techniques known to the skilled artisan, including, but not limited to, chromatographic, electrophoretic and centrifugation techniques.

### III. Anti-pNC Antibodies

In another aspect, the invention provides antibody molecules, e.g., antibodies and antigen-binding fragments thereof, which interact with, or more preferably specifically bind to pNC polypeptides or fragments thereof. In one embodiment, the antibody molecules bind to mNC and other chitinases, e.g., human AMCase or human chitotriosidase. In other embodiments, the antibody molecules bind specifically to a pNC polypeptide and does not substantially cross-reacts with other chitinases (e.g., binds to other chitinases with an affinity that is less than 50%, 40%, 30%, 20%, 10%, 5% or less compared to the affinity of the antibody molecule to the pNC). In one embodiment, the antibody molecule binds to an pNC epitope located on a catalytic a catalytic domain (e.g., about amino acids 22 to 408 of SEQ ID NO:2 or about amino acid 22 to 408 of SEQ ID NO:4), a linker (e.g., about amino acids 409 to 427 of SEQ ID NO:2 or about amino acids 409 to 435 of SEQ ID NO:4), and/or a chitin-binding domain (e.g., about amino acids 428 to 474 of SEQ ID NO:2 or about amino acid 436 to 482 of SEQ ID NO:4). In another embodiment, the antibody molecules reduce or inhibit one or more activities of a chitinase, e.g., a pNC polyptide as described herein, and/or a human AMCase or a human chitotriosidase. For example, the antibody molecule can inhibit or reduce about 20, 50, 60, 70, 80, 85, 90, 95% of one or more activities of a chitinase, e.g., a pNC polyptide as described herein, and/or a human AMCase or a human chitotriosidase.

In one aspect, the invention features a method of providing an antibody molecule that specifically binds to a primate, e.g., a human, chitinase protein. The method includes: providing a non-human chitinase, e.g., a mNC, or fragment thereof (e.g., an antigen that comprises at least a portion of the mNC protein as described herein, the portion being homologous to (at least about 70, 75, 80, 85, 87, 90, 92, 94, 95, 96, 97, or 98% identical to) a corresponding portion of a human chitinase protein, but differing by at least one amino acid (e.g., at least one, two, three, four, five, six, seven, eight, or nine amino acids)); obtaining an antibody molecule that specifically binds to the non-human chitinase or fragment thereof; and evaluating if the antibody molecule specifically binds to the human chitinase protein, or evaluating efficacy of the antibody molecule in modulating, e.g., inhibiting, the activity of the human chitinase protein. The method can further include administering the antibody molecule to a subject, e.g., a human or non-human primate. In one embodiment, the human chitinase protein is a human AMCase or a human chitotriosidase. The non-human protein can be from a non-human primate, e.g., a rhesus monkey, a cynomolgus monkey, or a pigtail macaque. In embodiments, the non-human protein comprises, or consists of, a full-length mature or precursor sequence, or at least a portion of the mNC protein, as described herein. Exemplary mNC fragments that can be used as an immunogen comprise, or consist of, about amino acids 22-475, 22-408, 409-427, 428-474, or about amino acids 1-21, 22-120, 22-180, 22-240, 22-300, 22-360, 22-400, 120-150, 150-170, 170-180, 180-190, 190-220, 220-250, 250-270, 270-280, 280-290, 290-300, 300-320, 320-330, 330-340, 340-350, 350-360, 360-390, 390-400, 400-410, 410-420, 420-430, 430-440, 440-450, 450-460, 460-470, or 470-476, of SEQ ID NO:2. Exemplary hNC fragments that can be used as an immunogen comprise, or consist of, about amino acids 22-482, 22-408, 409-427, 428-482, or about amino acids 1-21, 22-120, 22-180, 22-240, 22-300, 22-360, 22-400, 120-150, 150-170, 170-180, 180-190, 190-220, 220-250, 250-270, 270-280, 280-290, 290-300, 300-320, 320-330, 330-340, 340-350, 350-360, 360-390, 390-400, 400-410, 410-420, 420-430, 430-440, 440-450, 450-460, 460-470, 470-476, or 472-482 of SEQ ID NO:4.

The invention also provides fragments that can be used as an immunogen that comprise, or consist of any one of SEQ ID NOs:42-47.

In one embodiment, the step of obtaining the antibody molecule comprises using a protein expression library, e.g., a phage or ribosome display library. For example, the library displays antibody molecules such as Fab's or scFv's. In one embodiment, the step of obtaining the antibody molecule comprises immunizing an animal using the non-human chitinase antigen as an immunogen. For example, the animal can be a rodent, e.g., a mouse or rat. The animal can be a transgenic animal that has at least one human immunoglobulin gene.

As used herein, the term “antibody molecule” refers to a protein comprising at least one immunoglobulin variable domain sequence. The term antibody molecule includes, for example, full-length, mature antibodies and antigen-binding fragments of an antibody. For example, an antibody molecule can include a heavy (H) chain variable domain sequence (abbreviated herein as VH), and a light (L) chain variable domain sequence (abbreviated herein as VL). In another example, an antibody molecule includes two heavy (H) chain variable domain sequences and two light (L) chain variable domain sequence, thereby forming two antigen binding sites. Examples of antigen-binding fragments include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment, which consists of a VH domain; (vi) a camelid or camelized variable domain; and (vii) a single chain Fv (scFv), see e.g., Bird et al. (1988) Science 242:423-426; and Huston et al. (1988) Proc. Natl. Acad. Sci. USA 85:5879-5883). These antibody fragments are obtained using conventional techniques known to those with skill in the art, and the fragments are screened for utility in the same manner as are intact antibodies.

The VH and VL regions can be further subdivided into regions of hypervariability, termed “complementarity determining regions” (CDR), interspersed with regions that are more conserved, termed “framework regions” (FR). The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modelling software. See, generally, e.g., Protein Sequence and Structure Analysis of Antibody Variable Domains. In: Antibody Engineering Lab Manual (Ed.: Duebel, S, and Kontermann, R., Springer-Verlag, Heidelberg). Generally, unless specifically indicated, the following definitions are used: AbM definition of CDR1 of the heavy chain variable domain and Kabat definitions for the other CDRs. In addition, embodiments of the invention described with respect to Kabat or AbM CDRs may also be implemented using Chothia hypervariable loops. Each VH and VL typically includes three CDRs and four FRs, arranged from amino-terminus to carboxy-terminus in the following order: FR1, CDR1, FR2, CDR2, FR3, CDR3, FR4.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding site” refers to the part of an antibody molecule that comprises determinants that form an interface that binds to the pNC or an epitope thereof. With respect to proteins (or protein mimetics), the antigen-binding site typically includes one or more loops (of at least four amino acids or amino acid mimics) that form an interface that binds to pNC. Typically, the antigen-binding site of an antibody molecule includes at least one or two CDRs, or more typically at least three, four, five or six CDRs.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

An “effectively human” protein is a protein that does not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

The term “antibody” includes intact molecules as well as functional fragments thereof, such as Fab, Fab′, F(ab′)₂, Fc, Fd, Fd′, Fv, single chain antibodies (scFv for example), single variable domain antibodies (Dab), diabodies (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The antibodies of the present invention can be monoclonal or polyclonal. The antibody can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. Constant regions of the antibodies can be altered, e.g., mutated, to modify the properties of the antibody (e.g., to increase or decrease one or more of: Fc receptor binding, antibody glycosylation, the number of cysteine residues, effector cell function, or complement function). Typically, the antibody specifically binds to a predetermined antigen, e.g., an antigen associated with a disorder, e.g., a neurodegenerative, metabolic, inflammatory, autoimmune and/or a malignant disorder.

Antibodies of the present invention can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. In one aspect of the invention, a single domain antibody can be derived from a variable region of the immunoglobulin found in fish, such as, for example, that which is derived from the immunoglobulin isotype known as Novel Antigen Receptor (NAR) found in the serum of shark. Methods of producing single domain antibodies dervied from a variable region of NAR (“IgNARs”) are described in WO 03/014161 and Streltsov (2005) Protein Sci. 14:2901-2909. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The anti-pNC antibody can be a polyclonal or a monoclonal antibody. In other embodiments, the antibody can be recombinantly produced, e.g., produced by phage display or by combinatorial methods.

Phage display and combinatorial methods for generating anti-pNC antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J. 12:725-734; Hawkins et al. (1992) J Mol Biol 226:889-896; Clackson et al. (1991) Nature 352:624-628; Gram et al. (1992)PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the anti-pNC antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Method of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An anti-pNC antibody can be one in which the variable region, or a portion thereof, e.g., the CDR's, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDR's (of heavy and or light immuoglobulin chains) replaced with a donor CDR. The antibody may be replaced with at least a portion of a non-human CDR or only some of the CDR's may be replaced with non-human CDR's. It is only necessary to replace the number of CDR's required for binding of the humanized antibody to a pNC or a fragment thereof. Preferably, the donor will be a rodent antibody, e.g., a rat or mouse antibody, and the recipient will be a human framework or a human consensus framework. Typically, the immunoglobulin providing the CDR's is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art. Humanized antibodies can be generated by replacing sequences of the Fv variable region which are not directly involved in antigen binding with equivalent sequences from human Fv variable regions. General methods for generating humanized antibodies are provided by Morrison, S. L., 1985, Science 229:1202-1207, by Oi et al., 1986, BioTechniques 4:214, and by Queen et al. U.S. Pat. No. 5,585,089, U.S. Pat. No. 5,693,761 and U.S. Pat. No. 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acid are known to those skilled in the art and, for example, may be obtained from a hybridoma producing an antibody against a pNC polypeptide or fragment thereof. The recombinant DNA encoding the humanized antibody, or fragment thereof, can then be cloned into an appropriate expression vector.

Humanized or CDR-grafted antibodies can be produced by CDR-grafting or CDR substitution, wherein one, two, or all CDR's of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibodies in which specific amino acids have been substituted, deleted or added. Preferred humanized antibodies have amino acid substitutions in the framework region, such as to improve binding to the antigen. For example, a humanized antibody will have framework residues identical to the donor framework residue or to another amino acid other than the recipient framework residue. To generate such antibodies, a selected, small number of acceptor framework residues of the humanized immunoglobulin chain can be replaced by the corresponding donor amino acids. Preferred locations of the substitutions include amino acid residues adjacent to the CDR, or which are capable of interacting with a CDR (see e.g., U.S. Pat. No. 5,585,089). Criteria for selecting amino acids from the donor are described in U.S. Pat. No. 5,585,089, e.g., columns 12-16 of U.S. Pat. No. 5,585,089, the e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

In one embodiment, an antibody can be made by immunizing with purified pNC antigen, or a fragment thereof, e.g., a fragment described herein, membrane associated antigen, tissue, e.g., crude tissue preparations, whole cells, preferably living cells, lysed cells, or cell fractions, e.g., membrane fractions.

The anti-pNC antibody can be a single chain antibody. A single-chain antibody (scFV) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target pNC protein.

In a preferred embodiment the antibody has: effector function; and can fix complement. In other embodiments the antibody does not; recruit effector cells; or fix complement.

In a preferred embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

An anti-pNC antibody (e.g., monoclonal antibody) can be used to isolate pNC by standard techniques, such as affinity chromatography or immunoprecipitation. Moreover, an anti-pNC antibody can be used to detect pNC protein (e.g., in a cellular lysate or cell supernatant) in order to evaluate the abundance and pattern of expression of the protein. Anti-pNC antibodies can be used diagnostically to monitor protein levels in tissue as part of a clinical testing procedure, e.g., to determine the efficacy of a given treatment regimen. Detection can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinyl amine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

The invention also includes a nucleic acid which encodes an anti-pNC antibody, e.g., an anti-pNC antibody described herein. Also included are vectors which include the nucleic acid and cells transformed with the nucleic acid, particularly cells which are useful for producing an antibody, e.g., mammalian cells, e.g., CHO or lymphatic cells.

The invention also includes cell lines, e.g., hybridomas, which make an anti-pNC antibody, e.g., and antibody described herein, and method of using said cells to make a pNC antibody.

Also featured are nucleic acids encoding the pNC sequence and variants thereof. The polypeptide can be used to provide a pNC binding agent that binds pNC, and optionally, also a chitinase from another species.

In one aspect, the invention features a method of providing a target binding molecule that specifically binds to pNC. For example, the target binding molecule is an antibody molecule. The method includes: providing a target protein that comprises at least a portion of non-human protein, the portion being homologous to (at least 70, 75, 80, 85, 87, 90, 92, 94, 95, 96, 97, 98% identical to) a corresponding portion of a human target protein, but differing by at least one amino acid (e.g., at least one, two, three, four, five, six, seven, eight, or nine amino acids); obtaining a binding agent that specifically binds to the antigen; and evaluating efficacy of the binding agent in modulating activity of the target protein. The method can further include administering the binding agent (e.g., antibody molecule) or a derivative (e.g., a humanized antibody molecule) to a human subject. In one embodiment, the target protein is pNC.

In one embodiment, the step of obtaining comprises using a protein expression library, as described above. For example, the library displays antibody molecules such as Fab's of scFv's. In one embodiment, the step of obtaining comprises immunizing an animal using the antigen as an immungen. For example, the animal can be a rodent, e.g., a mouse or rat. The animal can be a transgenic animal.

### IV. Chitinase Modulators

A wide variety of chitinase modulators (e.g., inhibitors) may be tested in the screening methods of the present invention. For example, small molecule compounds, synthetic small molecule chemicals, chitinase-like inhibitor, variant fusion proteins, nucleic acids such as antisense oligonucleotides, RNA inhibitors such as siRNA, ribozymes, and aptamers.

In one embodiment, the chitinase modulator (e.g., chitinase inhibitor) is a binding domain-immunoglobulin fusion protein which includes a binding domain polypeptide fused or otherwise connected to an immunoglobulin hinge or hinge-acting region polypeptide, which in turn is fused or otherwise connected to a region comprising one or more native or engineered constant regions from an immunoglobulin heavy chain, other than CH1, for example, the CH2 and CH3 regions of IgG and IgA, or the CH3 and CH4 regions of IgE. The binding domain-immunoglobulin fusion protein can further include a region that includes a native or engineered immunoglobulin heavy chain CH2 constant region polypeptide (or CH3 in the case of a construct derived in whole or in part from IgE) that is fused or otherwise connected to the hinge region polypeptide and a native or engineered immunoglobulin heavy chain CH3 constant region polypeptide (or CH4 in the case of a construct derived in whole or in part from IgE) that is fused or otherwise connected to the CH2 constant region polypeptide (or CH3 in the case of a construct derived in whole or in part from IgE). Typically, such binding domain-immunoglobulin fusion proteins are capable of at least one immunological activity selected from the group consisting of antibody dependent cell-mediated cytotoxicity, complement fixation, and/or binding to a target, for example, a pNC.

A chitinase-like inhibitor also encompasses a chemical compound that inhibits the activity of a chitinase-like molecule. Chitinase-like molecule inhibitors are known in the art, and some of the key critical elements of one class of chitinase-like molecule inhibitors have been defined (Spindler and Spindler-Barth, 1999, Chitin and Chitinases, Birkhauser Verlag Basel, Switzerland). Additionally, a chitinase-like molecule inhibitor encompasses a chemically modified compound, and derivatives, as is known to one of skill in the chemical arts. Examples of chitinase-like inhibitors include, but not limited to, allosamidin (Allosamidine, Carbohydrate Chemistry Industrial Research Limited, Lower Hutt, New Zealand, and Eli Lilly and Co., Greenfield, Ind.) and its derivatives (see, e.g., U.S. Pat. No. 5,413,991), glucoallosamidin A, glucoallosamidin B, methyl-N-demethylallosamidin (Nishimoto et al., 1991, J. Antibiotics 44:716-722) demethylallosamidin (U.S. Pat. No. 5,070,191), and didemthylallosamidin (Zhou et al., 1993, J. Antibiotics 46:1582-1588). Further contemplated chitinase-like molecule inhibitors include stylogaunidine and its derivatives (Kato et al., 1995, Tetrahedron. Lett. 36:2133-2136), dipeptide cyclo-(L-Arg-D-Pro) (Izumida et al., 1996, J. Antibiotics 49:76-80), divalent cations (e.g., Cu.sup.2+, Zn.sup.2+, and Hg.sup.2+) (Izumida et al., 1995, J. Mar. Biotechnol. 2:163-166; Funke and Spindler, 1989, Comp. Biochem Physiol. 94B:691-695), and riboflavin and flavin derivatives (International Publication No. WO 02/23991).

### V. Recombinant Expression Vectors, Host Cells and Genetically Engineered Cells

In another aspect, the invention includes, vectors, preferably expression vectors, containing a nucleic acid encoding a polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a pNC nucleic acid in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., pNC proteins, mutant forms of pNC proteins, fusion proteins, and the like).

The recombinant expression vectors of the invention can be designed for expression of pNC proteins in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be used in pNC activity assays, (e.g., direct assays or competitive assays described in detail below), or to generate antibodies specific for pNC proteins. In a preferred embodiment, a fusion protein expressed in a retroviral expression vector of the present invention can be used to infect bone marrow cells which are subsequently transplanted into irradiated recipients. The pathology of the subject recipient is then examined after sufficient time has passed (e.g., six weeks).

To maximize recombinant protein expression in E. coli is to express the protein in a host bacteria with an impaired capacity to proteolytically cleave the recombinant protein (Gottesman, S., (1990) Gene Expression Technology: Methods in Enzymology 185, Academic Press, San Diego, Calif. 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

The pNC expression vector can be a yeast expression vector, a vector for expression in insect cells, e.g., a baculovirus expression vector or a vector suitable for expression in mammalian cells.

When used in mammalian cells, the expression vector's control functions can be provided by viral regulatory elements. For example, commonly used promoters are derived from polyoma, Adenovirus 2, cytomegalovirus and Simian Virus 40.

In another embodiment, the promoter is an inducible promoter, e.g., a promoter regulated by a steroid hormone, by a polypeptide hormone (e.g., by means of a signal transduction pathway), or by a heterologous polypeptide (e.g., the tetracycline-inducible systems, “Tet-On” and “Tet-Off”; see, e.g., Clontech Inc., CA, Gossen and Bujard (1992) Proc. Natl. Acad. Sci. USA 89:5547, and Paillard (1989) Human Gene Therapy 9:983).

In another embodiment, the recombinant mammalian expression vector is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus.

Another aspect the invention provides a host cell which includes a nucleic acid molecule described herein, e.g., a pNC nucleic acid molecule within a recombinant expression vector or a pNC nucleic acid molecule containing sequences which allow it to homologously recombine into a specific site of the host cell's genome. The terms “host cell” and “recombinant host cell” are used interchangeably herein. Such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a pNC protein can be expressed in bacterial cells (such as E. coli), insect cells, yeast or mammalian cells (such as Chinese hamster ovary cells (CHO) or COS cells e.g., COS-7 cells, CV-1 origin SV40 cells; Gluzman (1981) Cell I 23:175-182). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell of the invention can be used to produce (i.e., express) a pNC protein. Accordingly, the invention further provides methods for producing a pNC protein using the host cells of the invention. In one embodiment, the method includes culturing the host cell of the invention (into which a recombinant expression vector encoding a pNC protein has been introduced) in a suitable medium such that a pNC protein is produced. In another embodiment, the method further includes isolating a pNC protein from the medium or the host cell.

In another aspect, the invention features, a cell or purified preparation of cells which include a pNC transgene, or which otherwise misexpress pNC. The cell preparation can consist of human or non-human cells, e.g., rodent cells, e.g., mouse or rat cells, rabbit cells, or pig cells. In preferred embodiments, the cell or cells include a pNC transgene, e.g., a heterologous form of a pNC, e.g., a gene derived from humans (in the case of a non-human cell). The pNC transgene can be misexpressed, e.g., overexpressed or underexpressed. In other preferred embodiments, the cell or cells include a gene that mis-expresses an endogenous pNC, e.g., a gene the expression of which is disrupted, e.g., a knockout. Such cells can serve as a model for studying disorders that are related to mutated or mis-expressed pNC alleles or for use in drug screening.

Also provided are cells, preferably human cells, e.g., fibroblast cells, in which an endogenous pNC is under the control of a regulatory sequence that does not normally control the expression of the endogenous pNC gene. The expression characteristics of an endogenous gene within a cell, e.g., a cell line or microorganism, can be modified by inserting a heterologous DNA regulatory element into the genome of the cell such that the inserted regulatory element is operably linked to the endogenous pNC gene. For example, an endogenous pNC gene which is “transcriptionally silent,” e.g., not normally expressed, or expressed only at very low levels, may be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published in May 16, 1991.

In a preferred embodiment, recombinant cells described herein can be used for replacement therapy in a subject. For example, a nucleic acid encoding a pNC polypeptide operably linked to an inducible promoter (e.g., a steroid hormone receptor-regulated promoter) is introduced into a human or nonhuman, e.g., mammalian, e.g., porcine recombinant cell. The cell is cultivated and encapsulated in a biocompatible material, such as poly-lysine alginate, and subsequently implanted into the subject. See, e.g., Lanza (1996) Nat. Biotechnol. 14:1107; Joki et al. (2001) Nat. Biotechnol. 19:35; and U.S. Pat. No. 5,876,742. Production of pNC polypeptide can be regulated in the subject by administering an agent (e.g., a steroid hormone) to the subject. In another preferred embodiment, the implanted recombinant cells express and secrete an antibody specific for a pNC polypeptide. The antibody can be any antibody or any antibody derivative described herein.

vi. Transgenic Animals

The invention provides non-human transgenic animals. Such animals are useful for studying the function and/or activity of a pNC protein and for identifying and/or evaluating modulators of pNC activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA or a rearrangement, e.g., a deletion of endogenous chromosomal DNA, which preferably is integrated into or occurs in the genome of the cells of a transgenic animal. A transgene can direct the expression of an encoded gene product in one or more cell types or tissues of the transgenic animal, other transgenes, e.g., a knockout, reduce expression. Thus, a transgenic animal can be one in which an endogenous pNC gene has been altered by, e.g., by homologous recombination between the endogenous gene and an exogenous DNA molecule introduced into a cell of the animal, e.g., an embryonic cell of the animal, prior to development of the animal.

Intronic sequences and polyadenylation signals can also be included in the transgene to increase the efficiency of expression of the transgene. A tissue-specific regulatory sequence(s) can be operably linked to a transgene of the invention to direct expression of a pNC protein to particular cells. A transgenic founder animal can be identified based upon the presence of a pNC transgene in its genome and/or expression of pNC mRNA in tissues or cells of the animals. A transgenic founder animal can then be used to breed additional animals carrying the transgene. Moreover, transgenic animals carrying a transgene encoding a pNC protein can further be bred to other transgenic animals carrying other transgenes.

pNC proteins or polypeptides can be expressed in transgenic animals or plants, e.g., a nucleic acid encoding the protein or polypeptide can be introduced into the genome of an animal. In preferred embodiments the nucleic acid is placed under the control of a tissue specific promoter, e.g., a milk or egg specific promoter, and recovered from the milk or eggs produced by the animal. Suitable animals are mice, pigs, cows, goats, and sheep.

The invention also includes a population of cells from a transgenic animal, as discussed, e.g., below.

### VII. Uses

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic).

The isolated nucleic acid molecules of the invention can be used, for example, to express a pNC protein (e.g., via a recombinant expression vector in a host cell in gene therapy applications), to detect a pNC mRNA (e.g., in a biological sample) or a genetic alteration in a pNC gene, and to modulate pNC activity, as described further below. The pNC proteins can be used to treat disorders characterized by insufficient or excessive production of a pNC substrate or production of pNC inhibitors. In addition, the pNC proteins can be used to screen for naturally occurring pNC substrates, to screen for drugs or compounds which modulate pNC activity, as well as to treat disorders characterized by insufficient or excessive production of pNC protein or production of pNC protein forms which have decreased, aberrant or unwanted activity compared to pNC wild type protein. In one embodiment, as described above, the invention provides a method of screening for agents for treating asthma in a mammal by screening for an agent that modulates (e.g., inhibits or activates) the activity of pNC, including the expression levels of pNC. The method includes contacting a nucleotide sequence encoding a reporter gene product operably linked to an AMCase promoter, with a test agent; determining if the test agent inhibits production of the reporter gene product; and classifying the test agent as an agent for treating asthma if the test agent inhibits production of the reporter gene product. In one embodiment, the mammal is a human. “Asthma”, as used herein includes, but is not limited to, atopic asthma, nonatopic asthma, allergic asthma, exercise-induced asthma, drug-induced asthma, occupational asthma and late stage asthma. In another embodiment, the anti-pNC antibodies of the invention can be used to detect and isolate pNC proteins, regulate the bioavailability of pNC proteins, and modulate pNC activity.

A method of evaluating a compound for the ability to interact with, e.g., bind, a subject pNC polypeptide is provided. The method includes: contacting the compound with the subject pNC polypeptide; and evaluating ability of the compound to interact with, e.g., to bind or form a complex with the subject pNC polypeptide. This method can be performed in vitro, e.g., in a cell free system, or in vivo, e.g., in a two-hybrid interaction trap assay. This method can be used to identify naturally occurring molecules that interact with subject pNC polypeptide. It can also be used to find natural or synthetic inhibitors of subject pNC polypeptide. Screening methods are discussed in more detail below.

### Screening Assays

The invention provides methods (also referred to herein as “screening assays”) for identifying modulators, i.e., candidate or test compounds or agents (e.g., proteins, peptides, peptidomimetics, peptoids, small molecules or other drugs) which bind to pNC proteins, have a stimulatory or inhibitory effect on, for example, pNC expression or pNC activity, or have a stimulatory or inhibitory effect on, for example, the expression or activity of a pNC substrate. Compounds thus identified can be used to modulate the activity of target gene products (e.g., pNC genes) in a therapeutic protocol, to elaborate the biological function of the target gene product, or to identify compounds that disrupt normal target gene interactions.

In one embodiment, the invention provides assays for screening candidate or test compounds which are substrates of a pNC protein or polypeptide or a biologically active portion thereof. In another embodiment, the invention provides assays for screening candidate or test compounds that bind to or modulate an activity of a pNC protein or polypeptide or a biologically active portion thereof.

In one embodiment, an activity of a pNC protein can be assayed as follows. The sample (e.g., tissue, cell culture, or amount of pNC protein) is typically contacted with a test agent for a time period sufficient to inhibit the activity or expression of the chitinase. This time period may vary depending on the nature of the test agent, the pNC, the activity or expression detection method selected, and the sample tissue selected. The skilled artisan without undue experimentation may readily determine such times. An exemplary test agent is one that binds to or otherwise decreases the catalytic activity of a pNC, although test agents that inhibit pNC activity or expression by, for example, binding to the substrate for pNC, or binding to a component of the signal pathway, such asIL-13R, IL-4R or IL-13 are also envisioned. An exemplary substrate for chitinase is chitin, although derivatives thereof that may participate in a reaction catalyzed by chitinase are also encompassed by the invention.

A wide variety of assays may be utilized to determine whether the test agent inhibits the activity of the AMCase. For example, the amount of reactants remaining and/or products formed in reactions catalyzed by pNC may be quantified. A non-limiting example of such a reaction is the conversion of chitin or a chitin-like compound to N-acetyl-D-glucosamine. Other reactions include, without limitation, the release of 4-methylumbelliferyl from 4-methylumbelliferyl-tri-N-acetyl chitotrioxide, 4-methylumbelliferyl-D-N,N′-diacetylchitobiose or 4-methylumbelliferyl-D-N,N′,N″-triacetylchitotriose; or the release of p-nitrophenylfrom p-nitrophenyl P-D-0-D-N,N′-diacetylchitobiose or p-nitrophenyl-D-N,N′,N″-triacetylchitotriose. To this end, the amount of chitin remaining after contacting pNC with the test agent as a function of time may be determined. Similarly, the amount of N-acetyl-D-glucosamine or 4-methylumbelliferyl or p-nitrophenyl produced after contacting chitinase with the test agent in the presence of, for example, chitin as a function of time may be determined. Various assays may be used to determine the quantity of these products and/or reactants. For example, colorimetric assays may be utilized to determine the quantity of N-acetyl-D-glucosamine as described in, for example, Reissig, J. L., J. Biol. Chers. 217:959-966, 1955. Alternatively, the amount of glucosamine may be determined by chromatographic methods known to the skilled artisan, including high performance liquid chromatography, as described in, for example, Ekblad, A. (1996) Plant and Soil 178:29-35. Fluorometric assays may be utilized to determine the quantity of 4-methylumbelliferylorp-nitrophenyl as described in, for example, U.S. Pat. No. 5,561,051 (Silverman); Houston, D. R., et al., PNAS 99:9127-9232, 2002; Hollak, C. E. M., et al., J. Clin. Invest. 93:1288-1292, 1994; and Hu et al., J. Biol. Chem. 271:19415-194520. Methods of quantitating chitin are known to the art, including use of various immunoassays, such as enzyme-linked immunosorbents assays. Such assays, and others, are discussed in Muzzarelli R. A. A. and M. G. Peter, eds., Chitin Handbook, European Chitin Society, Atec, Grottammare (1997). A wide variety of test agents may be tested in the screening methods of the present invention. For example, small molecule compounds, known in the art, synthetic small molecule chemicals, nucleic acids such as antisense oligonucleotides, RNA inhibitors such as siRNA, ribozymes, and aptamers, peptides and proteins such as hormones, antibodies, and portions thereof, may act as test agents.

In one non-limiting example, the three-dimensional structure of the active site of acidic mammalian chitinase is determined by crystallizing the complex formed by the enzyme and a known inhibitor. Rational drug design is then used to identify new test agents by making alterations in the structure of a known inhibitor or by designing small molecule compounds that bind to the active site of the enzyme. Similarly, the skilled artisan would recognize that rational drug design could also be used to design antagonists of IL-13R and/or IL-4R, which would also be useful in modulating the production of pNC. As discussed elsewhere herein, test agents include inhibitors of chitinase enzymatic activity as well as inhibitors of chitinase expression or production. Nonlimiting examples of inhibitors of chitinase activity include allosamidin argifin, argadin, and antibodies to pNC, small molecule inhibitors, and proteins and peptides such as hormones and cytokines. Inhibitors of chitinase production include, without limitation, test agents that inhibit the production of pNC mRNA or protein. Non-limiting examples of such test agents include antagonists of IL-13, siRNA, antisense nucleic acids, ribozymes, aptamers, neutralizing antibodies to IL-13, IL-13R and IL-4R. Antagonists of IL-13 include without limitation test agents that block the ability of IL-13 to bind to either the IL-13 receptor or the IL-4 receptor. Such antagonists include without limitation IL-13R-Fc, sIL-13Ra2-Fc, and neutralizing antibodies against IL-13, IL-13R or IL-4R. In one embodiment, the invention also provides a method of screening for agents for treating asthma in a mammal by screening for an agent that modulates (e.g., inhibits or activates) the activity of pNC, including the expression level of pNC. The method includes contacting a nucleotide sequence encoding a reporter gene product operably linked to an pNC promoter, with a test agent; determining if the test agent inhibits production of the reporter gene product; and classifying the test agent as an agent for treating asthma if the test agent inhibits production of the reporter gene product. In one embodiment, the mammal is a human. “Asthma,” as used herein includes, but is not limited to, atopic asthma, nonatopic asthma, allergic asthma, exercise-induced asthma, drug-induced asthma, occupational asthma and late stage asthma.

The test compounds of the present invention can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In one embodiment, an assay is a cell-based assay in which a cell which expresses a pNC protein or biologically active portion thereof is contacted with a test compound, and the ability of the test compound to modulate pNC activity is determined. Determining the ability of the test compound to modulate pNC activity can be accomplished by monitoring, for example, enzymatic degradation of chitin or chitin-like substrate. The cell, for example, can be of mammalian origin, e.g., human.

The ability of the test compound to modulate pNC binding to a compound, e.g., a pNC substrate, or to bind to pNC can also be evaluated. This can be accomplished, for example, by coupling the compound, e.g., the substrate, with a radioisotope or enzymatic label such that binding of the compound, e.g., the substrate, to pNC can be determined by detecting the labeled compound, e.g., substrate, in a complex. Alternatively, pNC could be coupled with a radioisotope or enzymatic label to monitor the ability of a test compound to modulate pNC binding to a pNC substrate in a complex. For example, compounds (e.g., pNC substrates) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a pNC substrate) to interact with pNC with or without the labeling of any of the interactants can be evaluated. For example, a microphysiometer can be used to detect the interaction of a compound with pNC without the labeling of either the compound or the pNC. McConnell, H. M. et al. Science 257:1906-1912, 1992. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and pNC.

In yet another embodiment, a cell-free assay is provided in which a pNC protein or biologically active portion thereof is contacted with a test compound and the ability of the test compound to bind to the pNC protein or biologically active portion thereof is evaluated. Preferred biologically active portions of the pNC proteins to be used in assays of the present invention include fragments which participate in interactions with non-pNC molecules, e.g., fragments with high surface probability scores.

Soluble and/or membrane-bound forms of isolated proteins (e.g., pNC proteins or biologically active portions thereof) can be used in the cell-free assays of the invention. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, TRITON® X-100, TRITON® X-114, THESIT®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FET) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means known in the art (e.g., using a fluorimeter).

In another embodiment, determining the ability of the pNC protein to bind to a target molecule can be accomplished using real-time Biomolecular Interaction Analysis (BIA) (see, e.g., Sjolander, S, and Urbaniczky, C. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize either pNC, an anti-pNC antibody or its target molecule to facilitate separation of complexed from uncomplexed forms of one or both of the proteins, as well as to accommodate automation of the assay. Binding of a test compound to a pNC protein, or interaction of a pNC protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/pNC fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or pNC protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of pNC binding or activity determined using standard techniques.

Other techniques for immobilizing either a pNC protein or a target molecule on matrices include using conjugation of biotin and streptavidin. Biotinylated pNC protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with pNC protein or target molecules but which do not interfere with binding of the pNC protein to its target molecule. Such antibodies can be derivatized to the wells of the plate, and unbound target or pNC protein trapped in the wells by antibody conjugation. Methods for detecting such complexes, in addition to those described above for the GST-immobilized complexes, include immunodetection of complexes using antibodies reactive with the pNC protein or target molecule, as well as enzyme-linked assays which rely on detecting an enzymatic activity associated with the pNC protein or target molecule.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., (1993) Trends Biochem Sci 18:284-7); chromatography (gel filtration chromatography, ion-exchange chromatography); electrophoresis (see, e.g., Ausubel, F. et al., eds. Current Protocols in Molecular Biology 1999, J. Wiley: New York); and immunoprecipitation (see, for example, Ausubel, F. et al., eds. (1999) Current Protocols in Molecular Biology, J. Wiley: New York). Such resins and chromatographic techniques are known to one skilled in the art (see, e.g., Heegaard, N. H., (1998) J Mol Recognit 11:141-8; Hage, D. S., and Tweed, S. A. (1997) J Chromatogr B Biomed Sci Appl. 699:499-525). Further, fluorescence energy transfer may also be conveniently utilized, as described herein, to detect binding without further purification of the complex from solution.

In a preferred embodiment, the assay includes contacting the pNC protein or biologically active portion thereof with a known compound which binds pNC to form an assay mixture, contacting the assay mixture with a test compound, and determining the ability of the test compound to interact with a pNC protein, wherein determining the ability of the test compound to interact with a pNC protein includes determining the ability of the test compound to preferentially bind to pNC or biologically active portion thereof, or to modulate the activity of a target molecule, as compared to the known compound.

The target gene products of the invention can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the target gene product. Such compounds can include, but are not limited to molecules such as antibodies, peptides, and small molecules. The preferred target genes/products for use in this embodiment are the pNC genes herein identified. In an alternative embodiment, the invention provides methods for determining the ability of the test compound to modulate the activity of a pNC protein through modulation of the activity of a downstream effector of a pNC target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

In yet another aspect, the pNC proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with pNC (“pNC-binding proteins” or “pNC-bp”) and are involved in pNC activity. Such pNC-bps can be activators or inhibitors of signals by the pNC proteins or pNC targets as, for example, downstream elements of a pNC-mediated signaling pathway.

The two-hybrid system is based on the modular nature of most transcription factors, which consist of separable DNA-binding and activation domains. Briefly, the assay utilizes two different DNA constructs. In one construct, the gene that codes for a pNC protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. (Alternatively the: pNC protein can be the fused to the activator domain.) If the “bait” and the “prey” proteins are able to interact, in vivo, forming a pNC-dependent complex, the DNA-binding and activation domains of the transcription factor are brought into close proximity. This proximity allows transcription of a reporter gene (e.g., lacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the pNC protein.

In another embodiment, modulators of pNC expression are identified. For example, a cell or cell free mixture is contacted with a candidate compound and the expression of pNC mRNA or protein evaluated relative to the level of expression of pNC mRNA or protein in the absence of the candidate compound. When expression of pNC mRNA or protein is greater in the presence of the candidate compound than in its absence, the candidate compound is identified as a stimulator of pNC mRNA or protein expression. Alternatively, when expression of pNC mRNA or protein is less (statistically significantly less) in the presence of the candidate compound than in its absence, the candidate compound is identified as an inhibitor of pNC mRNA or protein expression. The level of pNC mRNA or protein expression can be determined by methods described herein for detecting pNC mRNA or protein.

In another aspect, the invention pertains to a combination of two or more of the assays described herein. For example, a modulating agent can be identified using a cell-based or a cell free assay, and the ability of the agent to modulate the activity of a pNC protein can be confirmed in vivo, e.g., in an animal such as an animal model for inflammatory lung disease, such asthma.

This invention further pertains to novel agents identified by the above-described screening assays. Accordingly, it is within the scope of this invention to further use an agent identified as described herein (e.g., a pNC modulating agent, an antisense pNC nucleic acid molecule, a pNC-specific antibody, or a pNC-binding partner) in an appropriate animal model to determine the efficacy, toxicity, side effects, or mechanism of action, of treatment with such an agent. Furthermore, novel agents identified by the above-described screening assays can be used for treatments as described herein.

The screening methods of the invention are performed either in vitro (for example by monitoring pNC activity in a cell-based assay or in an enzymatic activity assay) or in vivo (for example by monitoring pNC activity or expression in tissue samples such as BAL after administering a test agent to a mammal). Exemplary mammals include without limitation, human, mouse, rat, and dog.

### Detection Assays

Portions or fragments of the nucleic acid sequences identified herein can be used as polynucleotide reagents. For example, these sequences can be used to: (i) map their respective genes on a chromosome e.g., to locate gene regions associated with genetic disease or to associate pNC with a disease; (ii) identify an individual from a minute biological sample (tissue typing); and (iii) aid in forensic identification of a biological sample. These applications are described in the subsections below.

### Diagnostic and Prognostic Assays

Diagnostic and prognostic assays of the invention include method for assessing the expression level of pNC molecules and for identifying variations and mutations in the sequence of pNC molecules.

### Expression Monitoring and Profiling.

The presence, level, or absence of pNC protein or nucleic acid in a biological sample can be evaluated by obtaining a biological sample from a test subject and contacting the biological sample with a compound or an agent capable of detecting pNC protein or nucleic acid (e.g., mRNA, genomic DNA) that encodes pNC protein such that the presence of pNC protein or nucleic acid is detected in the biological sample. The term “biological sample” includes tissues, cells and biological fluids isolated from a subject, as well as tissues, cells and fluids present within a subject. A preferred biological sample is serum. The level of expression of the pNC gene can be measured in a number of ways, including, but not limited to: measuring the mRNA encoded by the pNC genes; measuring the amount of protein encoded by the pNC genes; or measuring the activity of the protein encoded by the pNC genes.

The level of mRNA corresponding to the pNC gene in a cell can be determined both by in situ and by in vitro formats.

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length pNC nucleic acid, such as the nucleic acid of SEQ ID NO:1, or a portion thereof, such as an oligonucleotide of at least 7, 15, 30, 50, 100, 250 or 500 nucleotides in length and sufficient to specifically hybridize under stringent conditions to pNC mRNA or genomic DNA. The probe can be disposed on an address of an array, e.g., an array described below. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array described below. A skilled artisan can adapt known mRNA detection methods for use in detecting the level of mRNA encoded by the pNC genes.

The level of mRNA in a sample that is encoded by one of pNC can be evaluated with nucleic acid amplification, e.g., by rtPCR (Mullis (1987) U.S. Pat. No. 4,683,202), ligase chain reaction (Barany (1991) Proc. Natl. Acad. Sci. USA 88:189-193), self sustained sequence replication (Guatelli et al., (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al. U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the pNC gene being analyzed.

In another embodiment, the methods further contacting a control sample with a compound or agent capable of detecting pNC mRNA, or genomic DNA, and comparing the presence of pNC mRNA or genomic DNA in the control sample with the presence of pNC mRNA or genomic DNA in the test sample. In still another embodiment, serial analysis of gene expression, as described in U.S. Pat. No. 5,695,937, is used to detect pNC transcript levels.

A variety of methods can be used to determine the level of protein encoded by pNC. In general, these methods include contacting an agent that selectively binds to the protein, such as an antibody with a sample, to evaluate the level of protein in the sample. In a preferred embodiment, the antibody bears a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods can be used to detect pNC protein in a biological sample in vitro as well as in vivo. In vitro techniques for detection of pNC protein include enzyme linked immunosorbent assays (ELISAs), immunoprecipitations, immunofluorescence, enzyme immunoassay (EIA), radioimmunoassay (RIA), and Western blot analysis. In vivo techniques for detection of pNC protein include introducing into a subject a labeled anti-pNC antibody. For example, the antibody can be labeled with a radioactive marker whose presence and location in a subject can be detected by standard imaging techniques. In another embodiment, the sample is labeled, e.g., biotinylated and then contacted to the antibody, e.g., an anti-pNC antibody positioned on an antibody array (as described below). The sample can be detected, e.g., with avidin coupled to a fluorescent label.

In another embodiment, the methods further include contacting the control sample with a compound or agent capable of detecting pNC protein, and comparing the presence of pNC protein in the control sample with the presence of pNC protein in the test sample.

The invention also includes kits for detecting the presence of pNC in a biological sample. For example, the kit can include a compound or agent capable of detecting pNC protein or mRNA in a biological sample; and a standard. The compound or agent can be packaged in a suitable container. The kit can further comprise instructions for using the kit to detect pNC protein or nucleic acid.

For antibody-based kits, the kit can include: (1) a first antibody (e.g., attached to a solid support) which binds to a polypeptide corresponding to a marker of the invention; and, optionally, (2) a second, different antibody which binds to either the polypeptide or the first antibody and is conjugated to a detectable agent.

For oligonucleotide-based kits, the kit can include: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also includes a buffering agent, a preservative, or a protein stabilizing agent. The kit can also includes components necessary for detecting the detectable agent (e.g., an enzyme or a substrate). The kit can also contain a control sample or a series of control samples which can be assayed and compared to the test sample contained. Each component of the kit can be enclosed within an individual container and all of the various containers can be within a single package, along with instructions for interpreting the results of the assays performed using the kit.

The diagnostic methods described herein can identify subjects having, or at risk of developing, a disease or disorder associated with misexpressed or aberrant or unwanted pNC expression or activity. As used herein, the term “unwanted” includes an unwanted phenomenon involved in a biological response such as airway inflammation.

In one embodiment, a disease or disorder associated with aberrant or unwanted pNC expression or activity is identified. A test sample is obtained from a subject and pNC protein or nucleic acid (e.g., mRNA or genomic DNA) is evaluated, wherein the level, e.g., the presence or absence, of pNC protein or nucleic acid is diagnostic for a subject having or at risk of developing a disease or disorder associated with aberrant or unwanted pNC expression or activity. As used herein, a “test sample” refers to a biological sample obtained from a subject of interest, including a biological fluid (e.g., serum), cell sample, or tissue.

The prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted pNC expression or activity. For example, such methods can be used to determine whether a subject can be effectively treated with an agent to antagonize or otherwise inhibit pNC expression or activity.

In another aspect, the invention features a computer medium having a plurality of digitally encoded data records. Each data record includes a value representing the level of expression of pNC in a sample, and a descriptor of the sample. The descriptor of the sample can be an identifier of the sample, a subject from which the sample was derived (e.g., a patient), a diagnosis, or a treatment (e.g., a preferred treatment). In a preferred embodiment, the data record further includes values representing the level of expression of genes other than pNC (e.g., other genes associated with a pNC-disorder, or other genes on an array). The data record can be structured as a table, e.g., a table that is part of a database such as a relational database (e.g., a SQL database of the Oracle or Sybase database environments).

Also featured is a method of evaluating a sample. The method includes providing a sample, e.g., from the subject, and determining a gene expression profile of the sample, wherein the profile includes a value representing the level of pNC expression. The method can further include comparing the value or the profile (i.e., multiple values) to a reference value or reference profile. The gene expression profile of the sample can be obtained by any of the methods described herein (e.g., by providing a nucleic acid from the sample and contacting the nucleic acid to an array). The method can be used to diagnose a airway inflammatory disorder in a subject wherein an increase in pNC expression or activity is an indication that the subject has or is disposed to having a chronic airway inflammatory disorder, including, for example, asthma. The method can be used to monitor a treatment for asthma in a subject. For example, the gene expression profile can be determined for a sample from a subject undergoing treatment. The profile can be compared to a reference profile or to a profile obtained from the subject prior to treatment or prior to onset of the disorder (see, e.g., Golub et al. (1999) Science 286:531).

In yet another aspect, the invention features a method of evaluating a test compound (see also, “Screening Assays”, above). The method includes providing a cell and a test compound; contacting the test compound to the cell; obtaining a subject expression profile for the contacted cell; and comparing the subject expression profile to one or more reference profiles. The profiles include a value representing the level of pNC expression. In a preferred embodiment, the subject expression profile is compared to a target profile, e.g., a profile for a normal cell or for desired condition of a cell. The test compound is evaluated favorably if the subject expression profile is more similar to the target profile than an expression profile obtained from an uncontacted cell.

In another aspect, the invention features, a method of evaluating a subject. The method includes: a) obtaining a sample from a subject, e.g., from a caregiver, e.g., a caregiver who obtains the sample from the subject; b) determining a subject expression profile for the sample. Optionally, the method further includes either or both of steps: c) comparing the subject expression profile to one or more reference expression profiles; and d) selecting the reference profile most similar to the subject reference profile. The subject expression profile and the reference profiles include a value representing the level of pNC expression. A variety of routine statistical measures can be used to compare two reference profiles. One possible metric is the length of the distance vector that is the difference between the two profiles. Each of the subject and reference profile is represented as a multi-dimensional vector, wherein each dimension is a value in the profile.

The method can further include transmitting a result to a caregiver. The result can be the subject expression profile, a result of a comparison of the subject expression profile with another profile, a most similar reference profile, or a descriptor of any of the aforementioned. The result can be transmitted across a computer network, e.g., the result can be in the form of a computer transmission, e.g., a computer data signal embedded in a carrier wave.

Also featured is a computer medium having executable code for effecting the following steps: receive a subject expression profile; access a database of reference expression profiles; and either i) select a matching reference profile most similar to the subject expression profile or ii) determine at least one comparison score for the similarity of the subject expression profile to at least one reference profile. The subject expression profile, and the reference expression profiles each include a value representing the level of pNC expression.

### Arrays and Uses Thereof

In another aspect, the invention features an array that includes a substrate having a plurality of addresses. At least one address of the plurality includes a capture probe that binds specifically to a pNC molecule (e.g., a pNC nucleic acid or a pNC polypeptide). The array can have a density of at least than 10, 50, 100, 200, 500, 1,000, 2,000, or 10,000 or more addresses/cm², and ranges between. In a preferred embodiment, the plurality of addresses includes at least 10, 100, 500, 1,000, 5,000, 10,000, 50,000 addresses. In a preferred embodiment, the plurality of addresses includes equal to or less than 10, 100, 500, 1,000, 5,000, 10,000, or 50,000 addresses. The substrate can be a two-dimensional substrate such as a glass slide, a wafer (e.g., silica or plastic), a mass spectroscopy plate, or a three-dimensional substrate such as a gel pad. Addresses in addition to address of the plurality can be disposed on the array.

In a preferred embodiment, at least one address of the plurality includes a nucleic acid capture probe that hybridizes specifically to a pNC nucleic acid, e.g., the sense or anti-sense strand. In one preferred embodiment, a subset of addresses of the plurality of addresses has a nucleic acid capture probe for pNC. Each address of the subset can include a capture probe that hybridizes to a different region of a pNC nucleic acid. In another preferred embodiment, addresses of the subset include a capture probe for a pNC nucleic acid. Each address of the subset is unique, overlapping, and complementary to a different variant of pNC (e.g., an allelic variant, or all possible hypothetical variants). The array can be used to sequence pNC by hybridization (see, e.g., U.S. Pat. No. 5,695,940).

An array can be generated by various methods, e.g., by photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT US/93/04145).

In another preferred embodiment, at least one address of the plurality includes a polypeptide capture probe that binds specifically to a pNC polypeptide or fragment thereof. The polypeptide can be a naturally-occurring interaction partner of pNC polypeptide. Preferably, the polypeptide is an antibody, e.g., an antibody described herein (see “Anti-pNC Antibodies,” above), such as a monoclonal antibody or a single-chain antibody.

In another aspect, the invention features a method of analyzing the expression of pNC. The method includes providing an array as described above; contacting the array with a sample and detecting binding of a pNC-molecule (e.g., nucleic acid or polypeptide) to the array. In a preferred embodiment, the array is a nucleic acid array. Optionally the method further includes amplifying nucleic acid from the sample prior or during contact with the array.

In another embodiment, the array can be used to assay gene expression in a tissue to ascertain tissue specificity of genes in the array, particularly the expression of pNC. If a sufficient number of diverse samples is analyzed, clustering (e.g., hierarchical clustering, k-means clustering, Bayesian clustering and the like) can be used to identify other genes which are co-regulated with pNC. For example, the array can be used for the quantitation of the expression of multiple genes. Thus, not only tissue specificity, but also the level of expression of a battery of genes in the tissue is ascertained. Quantitative data can be used to group (e.g., cluster) genes on the basis of their tissue expression per se and level of expression in that tissue.

For example, array analysis of gene expression can be used to assess the effect of cell-cell interactions on pNC expression. A first tissue can be perturbed and nucleic acid from a second tissue that interacts with the first tissue can be analyzed. In this context, the effect of one cell type on another cell type in response to a biological stimulus can be determined, e.g., to monitor the effect of cell-cell interaction at the level of gene expression.

In another embodiment, cells are contacted with a therapeutic agent. The expression profile of the cells is determined using the array, and the expression profile is compared to the profile of like cells not contacted with the agent. For example, the assay can be used to determine or analyze the molecular basis of an undesirable effect of the therapeutic agent. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor expression of one or more genes in the array with respect to time. For example, samples obtained from different time points can be probed with the array. Such analysis can identify and/or characterize the development of a pNC-associated disease or disorder; and processes, such as a cellular transformation associated with a pNC-associated disease or disorder. The method can also evaluate the treatment and/or progression of a pNC-associated disease or disorder

The array is also useful for ascertaining differential expression patterns of one or more genes in normal and abnormal cells. This provides a battery of genes (e.g., including pNC) that could serve as a molecular target for diagnosis or therapeutic intervention.

In another aspect, the invention features an array having a plurality of addresses. Each address of the plurality includes a unique polypeptide. At least one address of the plurality has disposed thereon a pNC polypeptide or fragment thereof. Methods of producing polypeptide arrays are described in the art, e.g., in De Wildt et al. (2000). Nature Biotech. 18, 989-994; Lueking et al. (1999). Anal. Biochem. 270, 103-111; Ge, H. (2000). Nucleic Acids Res. 28, e3, I-VII; MacBeath, G., and Schreiber, S. L. (2000). Science 289, 1760-1763; and WO 99/51773A1. In a preferred embodiment, each addresses of the plurality has disposed thereon a polypeptide at least 60, 70, 80, 85, 90, 95 or 99% identical to a pNC polypeptide or fragment thereof. For example, multiple variants of a pNC polypeptide (e.g., encoded by allelic variants, site-directed mutants, random mutants, or combinatorial mutants) can be disposed at individual addresses of the plurality. Addresses in addition to the address of the plurality can be disposed on the array.

The polypeptide array can be used to detect a pNC binding compound, e.g., an antibody in a sample from a subject with specificity for a pNC polypeptide or the presence of a pNC-binding protein or ligand.

The array is also useful for ascertaining the effect of the expression of a gene on the expression of other genes in the same cell or in different cells (e.g., ascertaining the effect of pNC expression on the expression of other genes). This provides, for example, for a selection of alternate molecular targets for therapeutic intervention if the ultimate or downstream target cannot be regulated.

In another aspect, the invention features a method of analyzing a plurality of probes. The method is useful, e.g., for analyzing gene expression. The method includes: providing a two dimensional array having a plurality of addresses, each address of the plurality being positionally distinguishable from each other address of the plurality having a unique capture probe, e.g., wherein the capture probes are from a cell or subject which express pNC or from a cell or subject in which a pNC mediated response has been elicited, e.g., by contact of the cell with pNC nucleic acid or protein, or administration to the cell or subject pNC nucleic acid or protein; providing a two dimensional array having a plurality of addresses, each address of the plurality being positionally distinguishable from each other address of the plurality, and each address of the plurality having a unique capture probe, e.g., wherein the capture probes are from a cell or subject which does not express pNC (or does not express as highly as in the case of the pNC positive plurality of capture probes) or from a cell or subject which in which a pNC mediated response has not been elicited (or has been elicited to a lesser extent than in the first sample); contacting the array with one or more inquiry probes (which is preferably other than a pNC nucleic acid, polypeptide, or antibody), and thereby evaluating the plurality of capture probes. Binding, e.g., in the case of a nucleic acid, hybridization with a capture probe at an address of the plurality, is detected, e.g., by signal generated from a label attached to the nucleic acid, polypeptide, or antibody.

In another aspect, the invention features a method of analyzing a plurality of probes or a sample. The method is useful, e.g., for analyzing gene expression. The method includes: providing a two dimensional array having a plurality of addresses, each address of the plurality being positionally distinguishable from each other address of the plurality having a unique capture probe, contacting the array with a first sample from a cell or subject which express or mis-express pNC or from a cell or subject in which a pNC-mediated response has been elicited, e.g., by contact of the cell with pNC nucleic acid or protein, or administration to the cell or subject pNC nucleic acid or protein; providing a two dimensional array having a plurality of addresses, each address of the plurality being positionally distinguishable from each other address of the plurality, and each address of the plurality having a unique capture probe, and contacting the array with a second sample from a cell or subject which does not express pNC (or does not express as highly as in the case of the pNC positive plurality of capture probes) or from a cell or subject which in which a pNC mediated response has not been elicited (or has been elicited to a lesser extent than in the first sample); and comparing the binding of the first sample with the binding of the second sample. Binding, e.g., in the case of a nucleic acid, hybridization with a capture probe at an address of the plurality, is detected, e.g., by signal generated from a label attached to the nucleic acid, polypeptide, or antibody. The same array can be used for both samples or different arrays can be used. If different arrays are used the plurality of addresses with capture probes should be present on both arrays.

In another aspect, the invention features a method of analyzing pNC, e.g., analyzing structure, function, or relatedness to other nucleic acid or amino acid sequences. The method includes: providing a pNC nucleic acid or amino acid sequence; comparing the pNC sequence with one or more preferably a plurality of sequences from a collection of sequences, e.g., a nucleic acid or protein sequence database; to thereby analyze pNC.

### Detection of Variations or Mutations

The methods of the invention can also be used to detect genetic alterations in a pNC gene, thereby determining if a subject with the altered gene is at risk for a disorder characterized by misregulation in pNC protein activity or nucleic acid expression, such as a chronic airway inflammatory disorder, including, for example, asthma. In preferred embodiments, the methods include detecting, in a sample from the subject, the presence or absence of a genetic alteration characterized by at least one of an alteration affecting the integrity of a gene encoding a pNC-protein, or the mis-expression of the pNC gene. For example, such genetic alterations can be detected by ascertaining the existence of at least one of 1) a deletion of one or more nucleotides from a pNC gene; 2) an addition of one or more nucleotides to a pNC gene; 3) a substitution of one or more nucleotides of a pNC gene, 4) a chromosomal rearrangement of a pNC gene; 5) an alteration in the level of a messenger RNA transcript of a pNC gene, 6) aberrant modification of a pNC gene, such as of the methylation pattern of the genomic DNA, 7) the presence of a non-wild type splicing pattern of a messenger RNA transcript of a pNC gene, 8) a non-wild type level of a pNC-protein, 9) allelic loss of a pNC gene, and 10) inappropriate post-translational modification of a pNC-protein.

An alteration can be detected without a probe/primer in a polymerase chain reaction, such as anchor PCR or RACE PCR, or, alternatively, in a ligation chain reaction (LCR), the latter of which can be particularly useful for detecting point mutations in the pNC-gene. This method can include the steps of collecting a sample of cells from a subject, isolating nucleic acid (e.g., genomic, mRNA or both) from the sample, contacting the nucleic acid sample with one or more primers which specifically hybridize to a pNC gene under conditions such that hybridization and amplification of the pNC-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein. Alternatively, other amplification methods described herein or known in the art can be used.

In another embodiment, mutations in a pNC gene from a sample cell can be identified by detecting alterations in restriction enzyme cleavage patterns. For example, sample and control DNA is isolated, amplified (optionally), digested with one or more restriction endonucleases, and fragment length sizes are determined, e.g., by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in pNC can be identified by hybridizing a sample and control nucleic acids, e.g., DNA or RNA, two-dimensional arrays, e.g., chip based arrays. Such arrays include a plurality of addresses, each of which is positionally distinguishable from the other. A different probe is located at each address of the plurality. A probe can be complementary to a region of a pNC nucleic acid or a putative variant (e.g., allelic variant) thereof. A probe can have one or more mismatches to a region of a pNC nucleic acid (e.g., a destabilizing mismatch). The arrays can have a high density of addresses, e.g., can contain hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in pNC can be identified in two-dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the pNC gene and detect mutations by comparing the sequence of the sample pNC with the corresponding wild-type (control) sequence. Automated sequencing procedures can be utilized when performing the diagnostic assays ((1995) Biotechniques 19:448), including sequencing by mass spectrometry.

Other methods for detecting mutations in the pNC gene include methods in which protection from cleavage agents is used to detect mismatched bases in RNA/RNA or RNA/DNA heteroduplexes (Myers et al. (1985) Science 230:1242; Cotton et al. (1988) Proc. Natl. Acad Sci USA 85:4397; Saleeba et al. (1992) Methods Enzymol. 217:286-295).

In still another embodiment, the mismatch cleavage reaction employs one or more proteins that recognize mismatched base pairs in double-stranded DNA (so called “DNA mismatch repair” enzymes) in defined systems for detecting and mapping point mutations in pNC cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662; U.S. Pat. No. 5,459,039).

In other embodiments, alterations in electrophoretic mobility will be used to identify mutations in pNC genes. For example, single strand conformation polymorphism (SSCP) may be used to detect differences in electrophoretic mobility between mutant and wild type nucleic acids (Orita et al. (1989) Proc Natl. Acad. Sci. USA: 86:2766, see also Cotton (1993) Mutat. Res. 285:125-144; and Hayashi (1992) Genet. Anal. Tech. Appl. 9:73-79). Single-stranded DNA fragments of sample and control pNC nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension (Saiki et al. (1986) Nature 324:163); Saiki et al. (1989) Proc. Natl. Acad. Sci USA 86:6230). A further method of detecting point mutations is the chemical ligation of oligonucleotides as described in Xu et al. ((2001) Nature Biotechnol. 19:148). Adjacent oligonucleotides, one of which selectively anneals to the query site, are ligated together if the nucleotide at the query site of the sample nucleic acid is complementary to the query oligonucleotide; ligation can be monitored, e.g., by fluorescent dyes coupled to the oligonucleotides.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

In another aspect, the invention features a set of oligonucleotides. The set includes a plurality of oligonucleotides, each of which is at least partially complementary (e.g., at least 50%, 60%, 70%, 80%, 90%, 92%, 95%, 97%, 98%, or 99% complementary) to a pNC nucleic acid.

In a preferred embodiment the set includes a first and a second oligonucleotide. The first and second oligonucleotide can hybridize to the same or to different locations of SEQ ID NO:1 or the complement of SEQ ID NO:1. Different locations can be different but overlapping, or non-overlapping on the same strand. The first and second oligonucleotide can hybridize to sites on the same or on different strands.

The set can be useful, e.g., for identifying SNP'S, or identifying specific alleles of pNC. In a preferred embodiment, each oligonucleotide of the set has a different nucleotide at an interrogation position. In one embodiment, the set includes two oligonucleotides, each complementary to a different allele at a locus, e.g., a biallelic or polymorphic locus.

In another embodiment, the set includes four oligonucleotides, each having a different nucleotide (e.g., adenine, guanine, cytosine, or thymidine) at the interrogation position. The interrogation position can be a SNP or the site of a mutation. In another preferred embodiment, the oligonucleotides of the plurality are identical in sequence to one another (except for differences in length). The oligonucleotides can be provided with differential labels, such that an oligonucleotide that hybridizes to one allele provides a signal that is distinguishable from an oligonucleotide that hybridizes to a second allele. In still another embodiment, at least one of the oligonucleotides of the set has a nucleotide change at a position in addition to a query position, e.g., a destabilizing mutation to decrease the Tₘ of the oligonucleotide. In another embodiment, at least one oligonucleotide of the set has a non-natural nucleotide, e.g., inosine. In a preferred embodiment, the oligonucleotides are attached to a solid support, e.g., to different addresses of an array or to different beads or nanoparticles.

In a preferred embodiment the set of oligo nucleotides can be used to specifically amplify, e.g., by PCR, or detect, a pNC nucleic acid.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a pNC gene.

### VIII. Methods of Treating Inflammatory Diseases

The present invention includes a method of treating an inflammatory disease wherein the disease is associated with an increased level of a chitinase-like molecule. Contemplated in the present invention are methods of treating an inflammatory disease in a mammal, preferably a human, using a chitinase-like molecule inhibitor (e.g., a chitinase inhibitor identified using the methods and assays disclosed herein).

A chitinase-like molecule inhibitor identified using the methods and assays disclosed herein can be administered to treat the disease even when there is no detectable chitinase activity. Thus, the skilled artisan would appreciate, based upon the disclosure provided herein, that the present invention is not limited to treatment of a disease where detectable chitinase activity is present; instead, the present invention encompasses treatment of a disease associated with or mediated by expression of a chitinase-like molecule even when there is no detectable chitinase activity.

It would be understood by one skilled in the art that inhibition of a chitinase-like molecule encompasses inhibition of a chitinase-like molecule expression, such as that mediated by, among other things, a ribozyme and/or antisense molecule that inhibits expression of a nucleic acid encoding a chitinase-like molecule. Additionally, inhibition of a chitinase-like molecule includes inhibition of a chitinase-like molecule activity in a cell. Such inhibition of a chitinase-like molecule activity can be effected using inhibitors of chitinase enzymatic activity, including, inter alia, allosamidin, 1,10-phenanthroline, glucoallosamidin A, glucoallosamidin B, methyl-N-demethylallosamidin, demethylallosamidin, didemthylallosamidin, stylogaunidine, a styloguanidine derivative, dipeptide cyclo-(L-Arg-D-Pro), dipeptide cyclo-(L-Arg-L-Pro), dipeptide cyclo-(D-Arg-D-Pro), dipeptide cyclo-(D-Arg-L-Pro), riboflavin, a flavin derivative, copper, zinc, mercury and the like. Further, inhibitors of chitinase-like molecule activity include an antibody molecule as described herein that specifically binds with a chitinase-like molecule thereby preventing the enzyme from functioning. Thus, a chitinase-like molecule inhibitor includes, but is not limited to, inhibiting transcription, translation, or both, of a nucleic acid encoding a chitinase-like molecule; and it also includes inhibiting any activity of the peptide as well, including, but not limited to, the ability to cleave chitin.

The present invention includes a method of treating or preventing an inflammatory disease in a mammal. The method comprises administering a chitinase-like molecule inhibitor to a mammal in need of such treatment. This is because, as would be appreciated by one skilled in the art armed with the teachings of the present invention, inhibiting a chitinase-like molecule is useful for treating or preventing an inflammatory disease. Inhibition of a chitinase-like molecule prevents, in turn, the pathology associated with an inflammatory disease.

More specifically, the invention relates to inhibiting a chitinase-like molecule using various inhibitors, e.g., an inhibitor identified using the methods and assays disclosed herein. That is, one skilled in the art would understand, based upon the disclosure provided herein, that compounds that inhibit the expression, activity, and/or function of a chitinase-like molecule encompass, but are not limited to, an antibody, an antisense nucleic acid, a ribozyme, a small molecule, a peptidomimetic and a chemical compound, either known or to be developed, which inhibits a chitinase-like molecule, and thereby an inflammatory disease.

One skilled in the art would appreciate that an inhibitor of the invention includes molecules and compounds that prevent or inhibit the expression, activity or function of a chitinase-like molecule in a mammal. That is, the invention contemplates that an antisense and/or antisense molecule that inhibits, decreases, and/or abolishes expression of a chitinase-like molecule such that the chitinase-like molecule is not detectable in the cell or tissue is an inhibitor of the invention. For instance, a compound that degrades a chitinase-like molecule can decrease its function, and can be an inhibitor as contemplated in the present invention.

Inhibition of a chitinase-like molecule can be assessed using a wide variety of methods, including those disclosed herein, as well as methods known in the art or to be developed in the future. That is, the routineer would appreciate, based upon the disclosure provided herein, that inhibition of chitinase-like molecule expression can be readily assessed using methods that assess the level of a nucleic acid encoding a chitinase-like molecule (e.g., mRNA) and/or the level of a chitinase-like molecule present in a cell or fluid. Moreover, the routineer would understand that inhibition of a chitinase-like molecule can be assessed by determining the inhibition of chitinase enzymatic activity in a cell or bodily fluid as exemplified elsewhere herein and/or using methods known in the art or to be developed in the future.

One skilled in the art would understand that the invention encompasses treatment of a variety of inflammatory diseases, including, but not limited to, asthma, chronic obstructive pulmonary disease, interstitial lung disease, chronic obstructive lung disease, chronic bronchitis, eosinophilic bronchitis, eosinophilic pneumonia, pneumonia, inflammatory bowel disease, atopic dermatitis, atopy, allergy, allergic rhinitis, idiopathic pulmonary fibrosis, scleroderma, and emphysema, and the like. As disclosed herein, these diseases involve and/or are mediated by, increased chitinase-like molecules in tissues where increased chitinase-like molecules includes, and is not limited to, increased chitinase-like molecule expression, increased chitinase-like molecule activity, or both. Further, the diseases encompass any disease comprising increased chitinase-like molecule in a tissue including, among others, a disease mediated by increased IL-13 and/or increased IL-4 production. This is because increased IL-13 and/or increased IL-4 mediates an increase in chitinase-like molecules which, in turns, mediates and/or is associated with a variety of changes associated with inflammatory disease including, but not limited to, tissue inflammation, increased lung volume, increased eosinophils in bronchioalveolar lavage (BAL) fluid, increased lymphocytes in BAL fluid, increased total cells in BAL fluid, increased alveolus size, increased deposition of crystals comprising chitinase-like molecules in lung tissue, increased airway resistance, increased mucus metaplasia, increased mucin expression, increased parenchymal fibrosis, increased airway remodeling, increased subepithelial fibrosis, increased collagen deposition in airway tissue, epithelial hypertrophy in the lung tissue, focal organization of crystalline material into Masson body-like fibrotic foci, and the like.

Further, the skilled artisan would appreciate that the term “chitinase associated diseases” encompasses any disease comprising increased chitinase-like molecule in a tissue including, among others, a disease mediated by increased Th2 inflammatory response. This is because, as more fully set forth elsewhere herein, the data disclosed herein demonstrate that increased Th2 inflammatory responses result in, inter alia, increased IL-13 and/or increased IL-4 activity and/or expression, an increase in chitinase-like molecules which, in turns, mediates and/or is associated with a variety of changes associated with inflammatory disease including, but not limited to, increased total cells in BAL fluid, increased alveolus size, increased deposition of crystals comprising chitinase-like molecules in lung tissue, increased airway resistance, increased mucus metaplasia, increased mucin expression, increased parenchymal fibrosis, increased airway remodeling, increased subepithelial fibrosis, increased eosinophils in bronchioalveolar lavage (BAL) fluid, increased lymphocytes in BAL fluid, and the like.

A chitinase-like molecule is a molecule that exhibits a substantial degree of homology to known chitinases, such that it has been or can be classified as a chitinase family molecule based upon, inter alia, its amino acid sequence. While a chitinase-like molecule can exhibit homology to a known chitinase, a chitinase-molecule need not demonstrate detectable chitinase activity, in that they may not detectably cleave chitin an in assay known in the art. Such chitinase like molecules include, but are not limited to, acidic mammalian chitinase (eosinophil chemotactic cytokine), YM1 (chitinase 3-like 3, ECF-L precursor), YM2, oviductal glycoprotein 1, cartilage glycoprotein 1 (BRP-39, chitinase 3-like 1, GP-39, YKL-40), chitotriosidase, oviductal glycoprotein 1 (mucin 9, oviductin), cartilage glycoprotein-39 (chitinase 3-like 1, GP-39, YKL-40), and chondrocyte protein 39 (chitinase 3-like 2, YKL-39).

A chitinase-like molecule inhibitor can include, but should not be construed as being limited to a chemical compound, a protein, a peptidomemetic, an antibody, a ribozyme, and an antisense nucleic acid molecule. Other examples of chitinase inhibitors are disclosed in Section IV, above.

Further methods of identifying and producing a chitinase-like molecule inhibitor are known to those of ordinary skill in the art, including, but not limited, obtaining an inhibitor from a naturally occurring source (i.e., Streptomyces sp., Pseudomonas sp., Stylotella aurantium). Alternatively, a chitinase-like molecule inhibitor can be synthesized chemically. Further, the routineer would appreciate, based upon the teachings provided herein, that a chitinase-like molecule inhibitor can be obtained from a recombinant organism. Compositions and methods for chemically synthesizing chitinase-like molecule inhibitors and for obtaining them from natural sources are known in the art and are described in, among others, Yamada et al., U.S. Pat. Nos. 5,413,991, and 5,070,191.

The skilled artisan would also appreciate, based on the disclosure provided herein, that a chitinase-like molecule inhibitor encompasses an antibody that specifically binds with a chitinase-like molecule, for example, AMCase, thereby inhibiting the action of these proteins. For instance, antibodies that specifically bind to YM are known to those of ordinary skill in the art (Webb et al., 2001, J. Biol. Chem. 276:41969-41976). Similarly, antibodies to chitinase-like molecules can be produced using standard methods disclosed herein or known to those of ordinary skill in the art (Harlow et al., 1988, Antibodies: A Laboratory Manual, Cold Spring Harbor, N.Y.). Thus, the present invention is not limited in any way to any particular antibody; instead, the invention includes any antibody that specifically binds with a chitinase-like molecule either known in the art and/or identified in the future.

One of skill in the art will appreciate that an antibody can be administered as a protein, a nucleic acid construct encoding a protein, or both. Numerous vectors and other compositions and methods are known for administering a protein or a nucleic acid construct encoding a protein to cells or tissues. Therefore, the invention includes a method of administering an antibody or nucleic acid encoding an antibody (e.g., synthetic antibody) that is specific for a chitinase-like molecule. (Sambrook et al., 1989, Molecular Cloning: A Laboratory Manual, Cold Spring Harbor Laboratory, New York; Ausubel et al., 1997, Current Protocols in Molecular Biology, John Wiley & Sons, New York).

The skilled artisan would understand, based upon the disclosure provided herein, that the invention encompasses administering an antibody that specifically binds with a chitinase-like molecule of interest, or a nucleic acid encoding the antibody, wherein the antibody molecule further comprises an intracellular retention sequence such that the antibody binds with the chitinase-like molecule and prevents its expression at the cell surface and/or its export from a cell. Such antibodies, frequently referred to as “intrabodies”, are known in the art and are described in, for example, Marasco et al. (U.S. Pat. No. 6,004,490) and Beerli et al. (1996, Breast Cancer Research and Treatment 38:11-17). Thus, the invention encompasses methods comprising inhibiting expression of a chitinase-like molecule on a cell and/or its secretion from a cell, where the skilled artisan would understand such inhibition would provide a benefit based upon the disclosure provided herein.

The present invention is not limited to chemical compounds and antibodies against a chitinase-like molecule. One of skill in the art would appreciate that inhibiting the expression of a polypeptide is like wise an effective method of inhibiting the activity and function of the polypeptide. Thus, a method is provided for the inhibition of a chitinase-like molecule by inhibiting the expression of a nucleic acid encoding a chitinase-like molecule. Methods to inhibit the expression of a gene are known to those of ordinary skill in the art, and include the use of ribozymes or antisense oligonucleotide, e.g., ribozymes or antisense oligonucleotides as described herein.

One of skill in the art will appreciate that inhibitors of chitinase-like molecule gene expression can be administered singly or in any combination thereof. Further, chitinase-like molecule inhibitors can be administered singly or in any combination thereof in a temporal sense, in that they may be administered simultaneously, before, and/or after each other. One of ordinary skill in the art will appreciate, based on the disclosure provided herein, that chitinase-like molecule inhibitors to inhibit gene expression can be used to treat asthma, COPD, and other inflammatory diseases and that an inhibitor can be used alone or in any combination with another inhibitor to effect a therapeutic result.

It will be appreciated by one of skill in the art, when armed with the present disclosure including the methods detailed herein, that the invention is not limited to treatment of an inflammatory disease once the disease is established. Particularly, the symptoms of the disease need not have manifested to the point of detriment to the mammal; indeed, the disease need not be detected in a mammal before treatment is administered. That is, significant pathology from an inflammatory disease does not have to occur before the present invention may provide benefit. Therefore, the present invention, as described more fully herein, includes a method for preventing an inflammatory disease in a mammal, in that a chitinase-like molecule inhibitor, as discussed previously elsewhere herein, can be administered to a mammal prior to the onset of an inflammatory disease, thereby preventing the disease as demonstrated by the data disclosed herein.

One of skill in the art would appreciate that the prevention of inflammatory disease encompasses administering to a mammal a chitinase-like molecule inhibitor as a preventative measure against inflammatory disease. As detailed herein, the symptoms and etiologies of chitinase-like molecule-associated inflammatory disease include tissue inflammation, increased lung volume, increased eosinophils in bronchioalveolar lavage (BAL) fluid, increased lymphocytes in BAL fluid, iricreased total cells in BAL fluid, increased alveolus size, increased deposition of crystals comprised of chitinase-like molecules in lung tissue, increased airway resistance, increased mucus metaplasia, increased mucin expression, increased parenchymal fibrosis, increased airway remodeling, increased subepithelial fibrosis, increased collagen deposition in airway tissue, epithelial hypertrophy in the lung tissue, focal organization of crystalline material into Masson body-like fibrotic foci, and the like.

The invention encompasses administration of a chitinase-like molecule inhibitor to practice the methods of the invention; the skilled artisan would understand, based on the disclosure provided herein, how to formulate and administer the appropriate chitinase-like molecule inhibitor to a mammal. Indeed, the successful administration of chitinase-like molecule inhibitors have been extensively reduced to practice as exemplified herein. However, the present invention is not limited to any particular method of administration or treatment regimen.

### IX. Pharmaceutical Compositions

The nucleic acid and polypeptides, fragments thereof, as well as anti-pNC antibodies, and small molecules (e.g., small molecule pNC inhibitors or activators) (also referred to herein as “active compounds”) of the invention can be incorporated into pharmaceutical compositions. Such compositions typically include the nucleic acid molecule, protein, or antibody and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

For antibodies, the preferred dosage is 0.1 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The present invention encompasses agents which modulate expression or activity. An agent may, for example, be a small molecule. For example, such small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

An antibody (or fragment thereof) may be conjugated to a therapeutic moiety such as a cytotoxin, a therapeutic agent or a radioactive ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020), CC-1065 (see U.S. Pat. Nos. 5,475,092, 5,585,499, 5,846,545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids). Radioactive ions include, but are not limited to iodine, yttrium and praseodymium.

The conjugates can be used for modifying a given biological response, the drug moiety is not to be construed as limited to classical chemical therapeutic agents. For example, the drug moiety may be a protein or polypeptide possessing a desired biological activity. Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

The Examples that follow are set forth to aid in the understanding of the inventions but are not intended to, and should not be construed to, limit its scope in any way.

### EXAMPLE

The Examples below show the cloning and characterization of two novel primate chitinases and nucleic acids encoding these chitinases, referred to herein collectively as “primate Novel Chitinases” or “pNCs,” or by their individual names “macaque Novel Chitinase” or “mNC,” and “human Novel Chitinase” or “hNC.” mNC was cloned from cynomolgus monkey. The hNC is a nucleic acid sequence based on a human pseudogene which has been engineered so that the deletion in exon 8 of the human chitinase genomic sequence is repaired and a sequence encoding a full length protein is obtained. FIGS. 1 and 2 show the nucleotide and amino acid sequences, respectively, derived for macaque novel chitinase, herein designated mNC. FIGS. 3A and 4A show the nucleotide and amino acid sequences, respectively, derived for the engineered human novel chitinase, herein designated hNC. FIGS. 3B and 4B show the nucleotide and the amino acid sequences, respectively, of the human chitinase pseudogene.

### Example 1

### Identification of Human Novel Chitinase

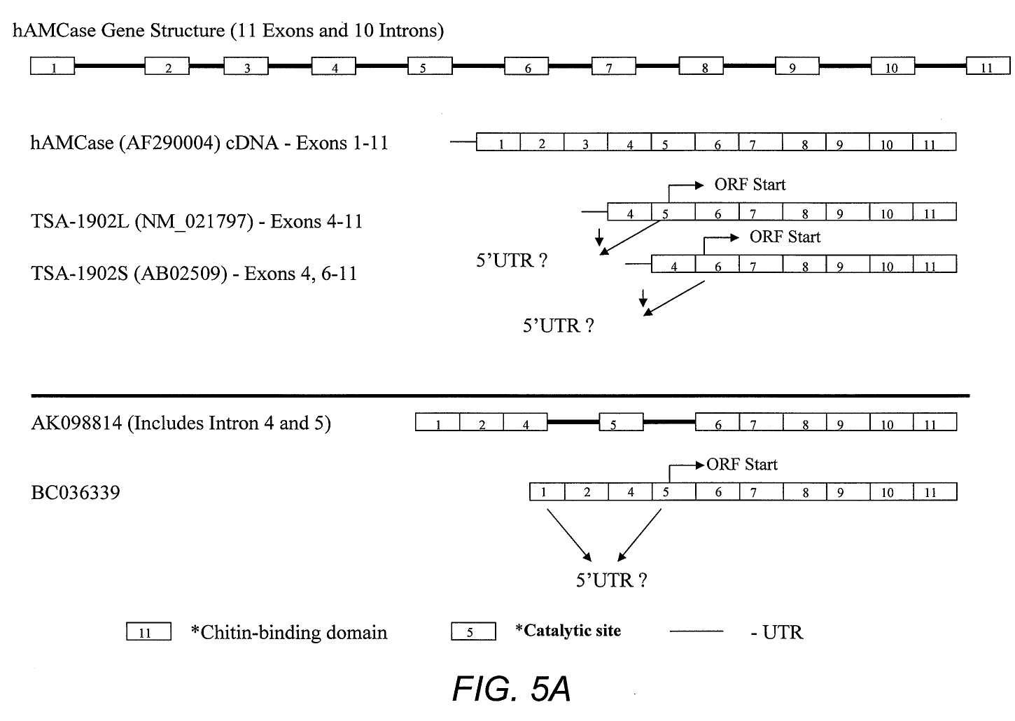

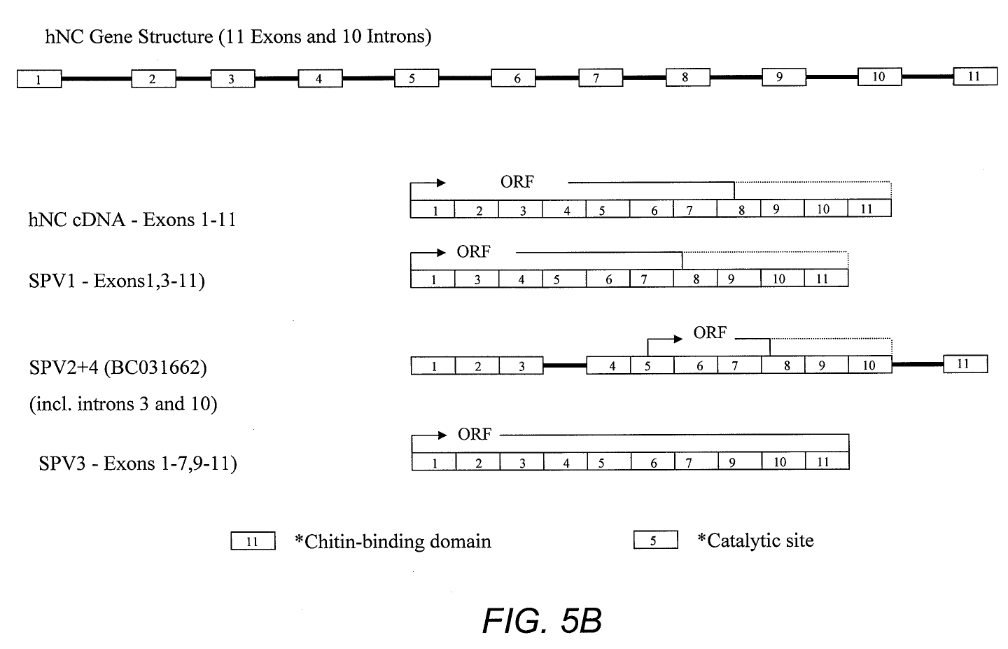

FIG. 5A (top) shows the gene structure of human AMCase (hAMCase) including exons 1 through 11, and introns 1 through 10, as well as several published AMCase full length and splice variants. Of those shown, only AMCase (AF290004 is reported to show enzymatic activity.

As shown in FIG. 5B (top), by in silico screening ensemble genomic database, EST, and Geneseqn sequences, a putative hNC was identified. Similar to hAMCase, this putative hNC was predicted to encode 11 exons and 10 introns. Analysis of this sequence revealed a stop codon in exon 8 (see FIG. 5B star) caused by a single nucleotide upstream frameshifting deletion. FIG. 5B also shows other available EST sequences, namely, SPV1, SPV2+4, SPV3.

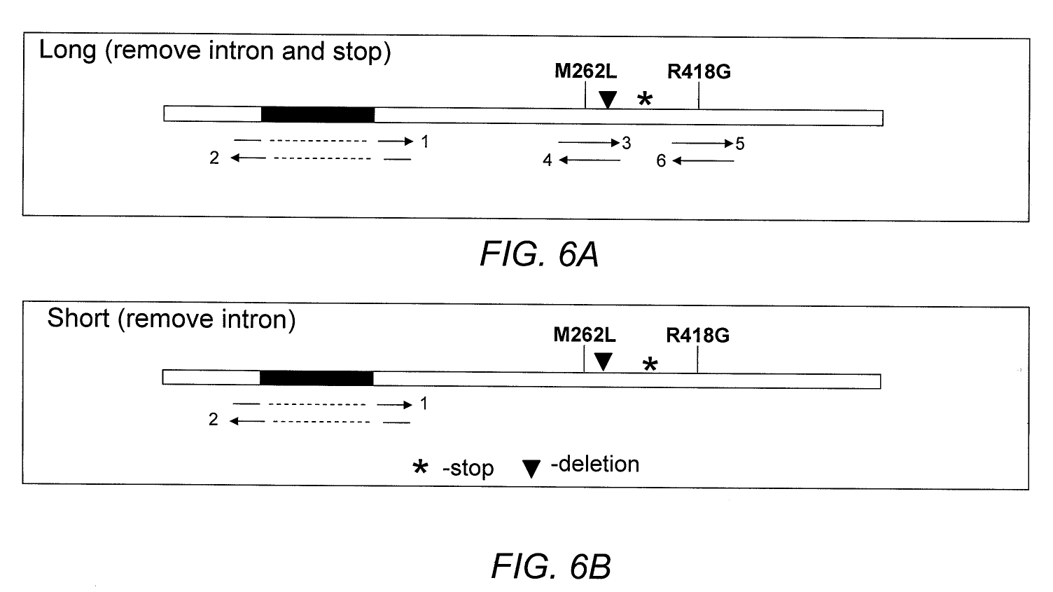

In an attempt to express this gene and verify its enzymatic activity, an hNC cDNA was engineered using a commercially available EST (LIFESEQ3645544; Openbiosystems), using the strategy illustrated in FIGS. 6A and 6B. The aforementioned stop codon was removed by replacing the above described single nucleotide frameshifting deletion to allow full length protein translation, as represented in FIG. 5B-hNC cDNA.

Briefly, two hNC constructs (see FIGS. 6A and 6B) were engineered to encode exons 1 to 11 as follows. A full-length hNC cDNA (FIG. 6A) was generated using, for example, SEQ ID NO: 5 and SEQ ID NO: 8. The intron sequence shown in FIG. 6A as a black shaded region was deleted using SEQ ID NO: 30 and SEQ ID NO: 31.

(SEQ ID NO: 30)

5′-GCTTTCAATGGCCTGAAAAACAAGAATAGTCAACTGAAAACTCTCTT
GGCTATTGG-3′

(SEQ ID NO: 31)

5′-CCAATAGCCAAGAGAGTTTTCAGTTGACTATTCTTGTTTTTCAGGCC
ATTGAAAGC-3′

An M262L substitution was then introduced to remove an EST error and the single nucleotide frameshifting deletion was corrected using SEQ ID NO: 32 and SEQ ID NO: 33.

(SEQ ID NO: 32)

5′-CCCCAGCTGAGAAGCTCTTGGTTGGATTCCCAGCCTATGGAC-3′

(SEQ ID NO: 33)

5′-GTCCATAGGCTGGGAATCCAACCAAGAGCTTCTCAGCTGGGG-3′

A R418G substitution was then introduced using SEQ ID NO: 34 and SEQ ID NO: 35.

(SEQ ID NO: 34)

5′-GCGGTGTCAGCCACAGTGGTAGCTCTGGGGGCCGCT-3′

(SEQ ID NO: 35)

5′-AGCGGCCCCCAGAGCTACCACTGTGGCTGACACCGC-3′

As shown in FIG. 6B, a truncated hNC construct was generated (mut-hNC) in which the intron sequence shown as a black shaded region was deleted using SEQ ID NO: 30 and SEQ ID NO: 31. As the single nucleotide frameshifting deletion was not replaced using SEQ ID NO: 32 and SEQ ID NO: 33, this construct is expected to be expressed as a truncated mutant. This second construct was generated to analyze whether a truncated hNC is enzymatically active. Both engineered hNC constructs were cloned into a cloning vector using GATEWAY® technology (Invitrogen).

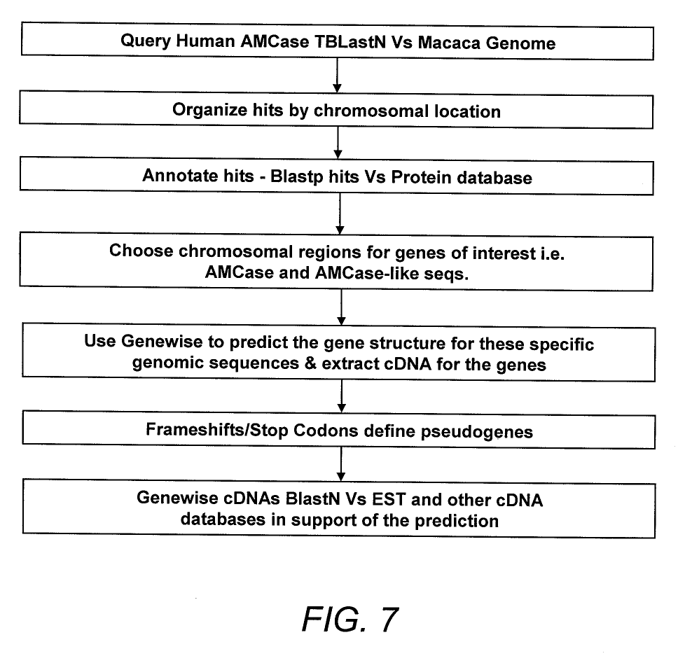

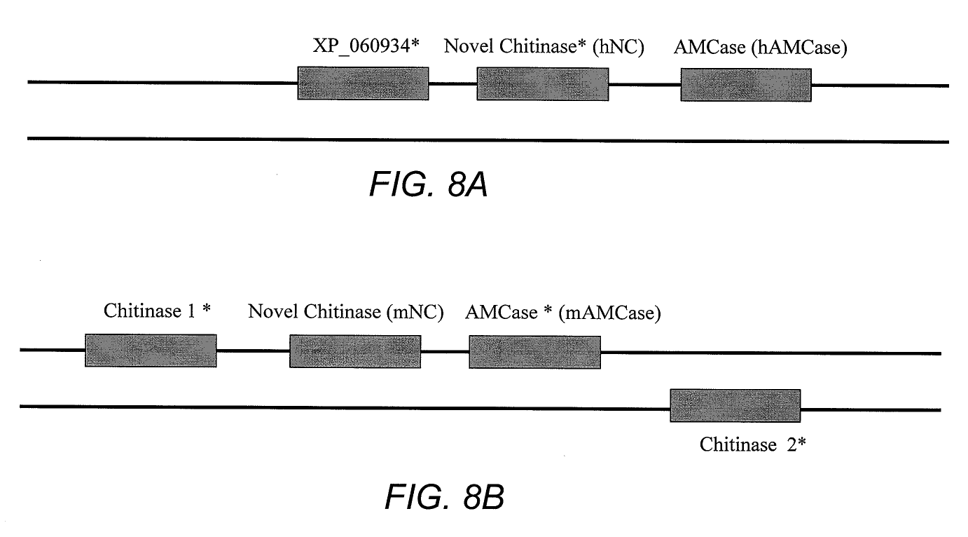

Additional in silico screenings were performed, according to the strategy illustrated in FIG. 7, to predict further human and monkey AMCase-like sequences from Homo Sapiens and Macaca Mulatta (Rhesus) genome databases. The results of these in silico screens are illustrated in FIG. 8.

As illustrated in FIG. 8A, using the in silico search strategy illustrated in FIG. 7, two novel human AMCase-like genes, designated herein as XP_{—}060934, and hNC (which is described above) were identified.

Detailed analysis of the XP_{—}060934 sequence identified a stop codon, strongly indicating that this gene is likely a pseudogene. As described supra, hNC also contains a stop codon in exon 8. hAMCase is a previously described enzymatically active chitinase (Boot R. G., et al., J. Biol. Chem., 276:6770-6778, 2001).

As illustrated in FIG. 8B, the in silico search strategy illustrated in FIG. 7 also predicted 4 novel monkey AMCase-like genes, which are designated herein as, chitinase 1*, mNC, mAMCase *, and chitinase 2*. Asterisk indicates frameshifting deletion in a sequence leading to a truncated protein sequence. Analysis of the sequences revealed that of the newly identified genes described above, chitinase 1* is 53% homologous to hAMCase. However, due to the detection of a stop codon, chitinase 1* is predicted to be a pseudogene. Chitinase 2* was observed to be 46% homologous to hAMCase, however, it does not encode a catalytic domain or a chitin binding domain and is thus believed to be non-functional.

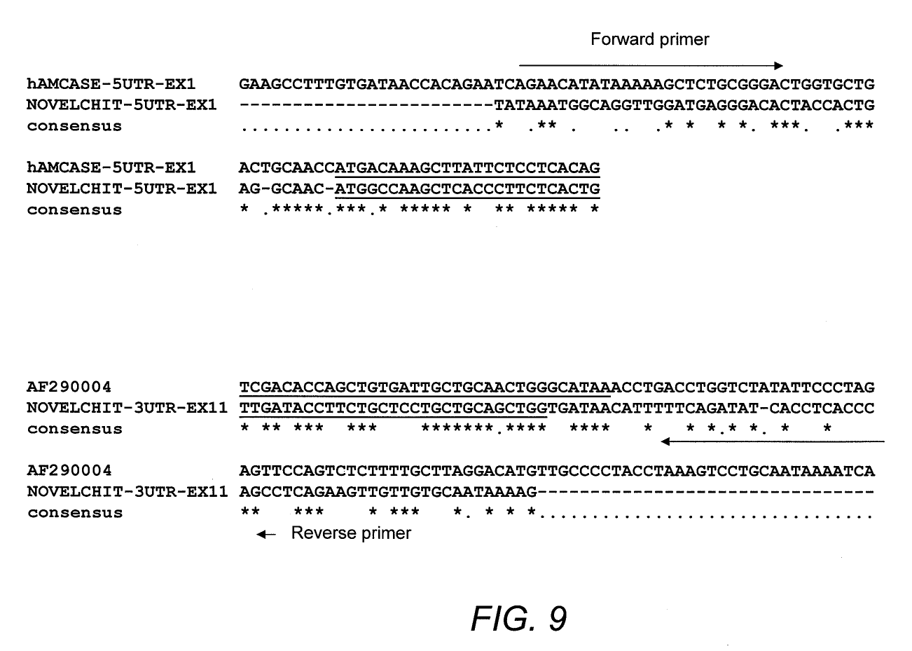

Due to the absence of a published monkey genome at the time of this study, mNC and macaque AMCase (mAMCase) genes were amplified using RT-PCR coupled with primers that were designed to be complementary to hAMCase (see SEQ ID NOs: 5 and 6, below) and to hNC, described above (see SEQ ID NOs: 7 and 8, below), respectively. SEQ ID NOs: 5 and 8 are represented in FIG. 9 as forward primer and reverse primer, respectively. As shown in FIG. 9, SEQ ID NOs: 5 and 8, as well as SEQ ID NOs: 6 and 7 may be used to clone hAMCase, mAMCase, hNC, and mNC. Also, shown in FIG. 9, the overall sequence homology was lower in the untranslated regions than the sequence homology in the coding region.

| | |
|---|---|
| 5′-TCAGAACATATAAAAAGCTCTGCGG-3′ | (SEQ ID NO: 5) |
| 5′-CTCTAGGGAATATAGACCAGGTCAGGT-3 | (SEQ ID NO: 6) |
| 5′-TATAAATGGCAGGTTGGATGAGGG-3′ | (SEQ ID NO: 7) |
| 5′-CTGGGTGAGGTGATATCTGAAAAATG-3′ | (SEQ ID NO: 8) |

Sequencing was performed of DNA fragments obtained by PCR using a combination of SEQ ID NO: 5 and SEQ ID NO: 6 to amplify the newly identified AMCase-like gene, and a combination of SEQ ID NO: 7 and SEQ ID NO:8 to amplify the novel chitinase.

mNC and mAMCase are highly homologous to human AMCase. mNC and mAMCase were further investigated as follows.

### Example 2

### Expression and Tissue Distribution of Primate Chitinases

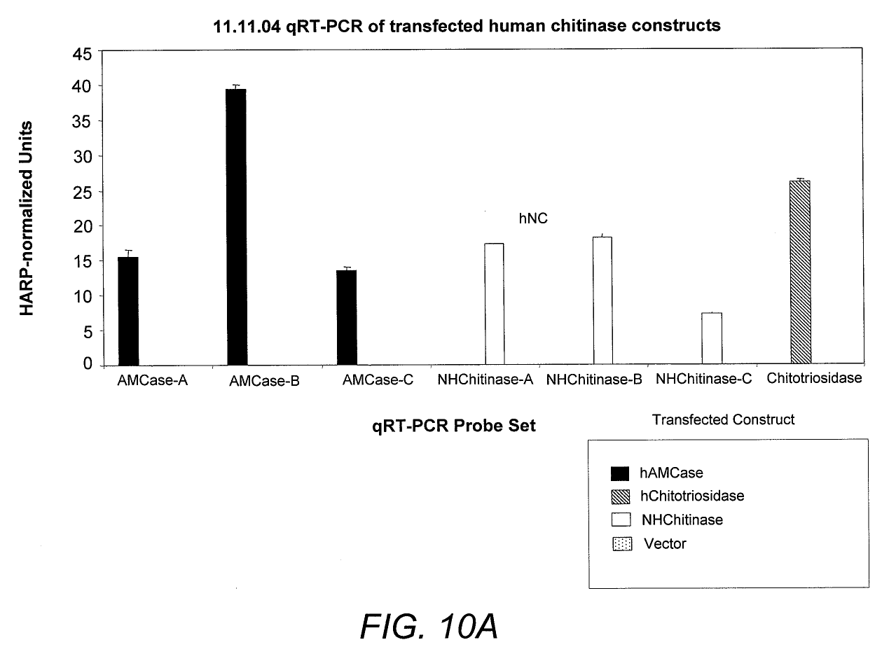

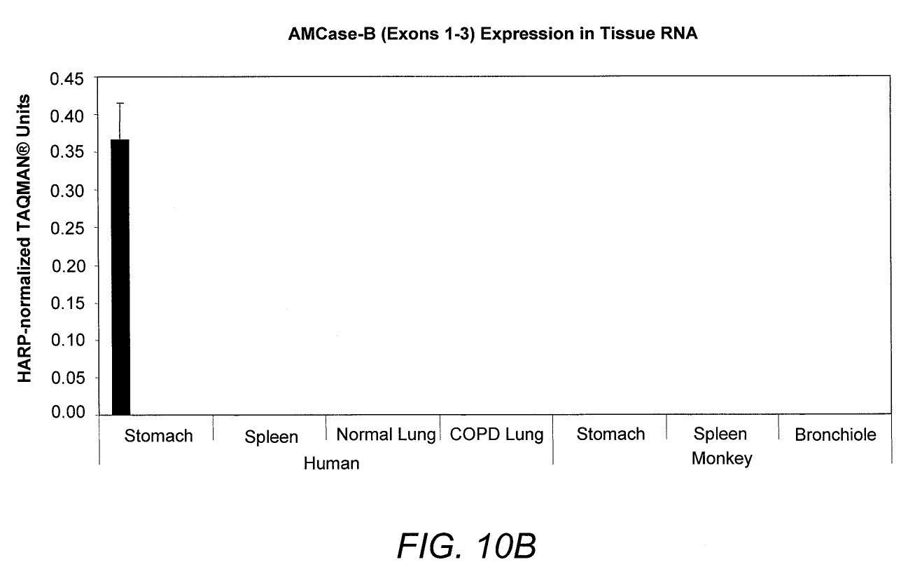

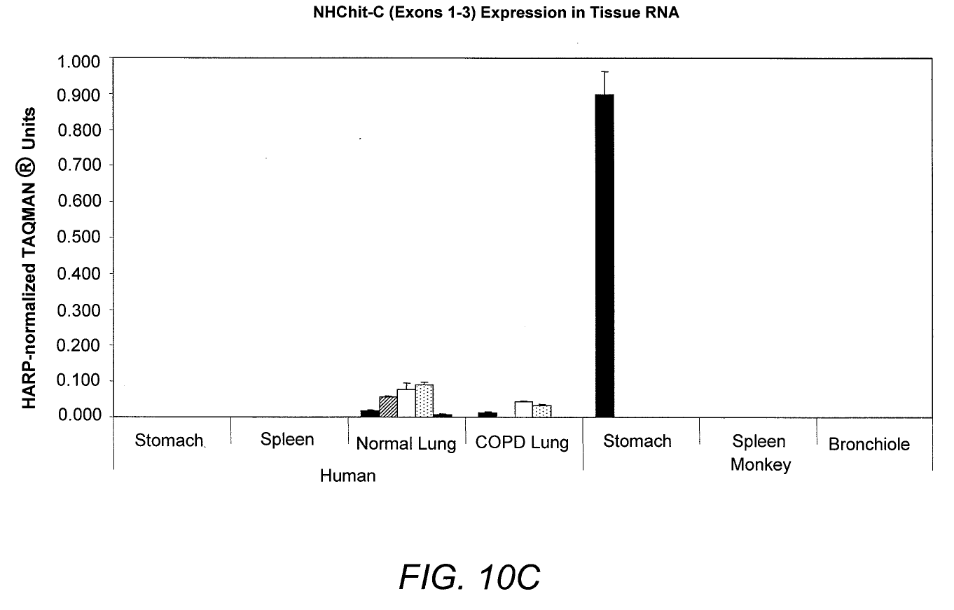

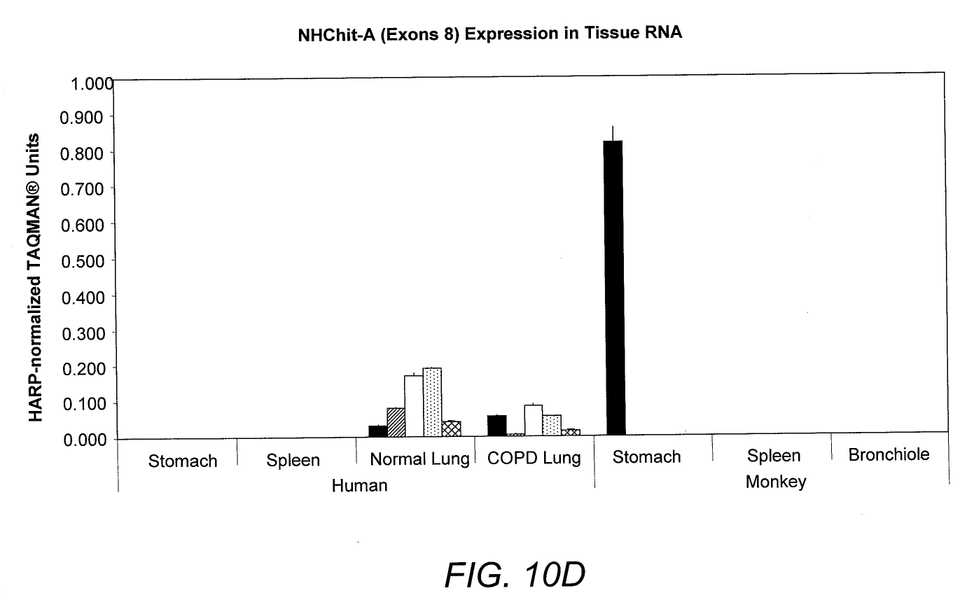

The expression levels of hNC and mNC in cells in culture and tissues were analyzed using qRT-PCR by TAQMAN®. The results are summarized in FIGS. 10A-10D, as follows: Human and macaque qRT-PCR data for transfected COS-M6 cells expressing hAMCase (hMCase), human chitotriosidase (hChitotriosidase), hNC, and vector is shown in FIG. 10A. FIG. 10B depicts endogenous mRNA expression levels of exons 1-3 of hAMCase and mAMCase. FIG. 10C shows endogenous mRNA expression levels of exons 1-3 of the putative hNC and mNC. FIG. 10D depicts endogenous expression levels of exon 8 mRNA of the putative hNC and mNC. More detailed experimental conditions and results are provided herein.

Briefly, TAQMAN® technology relies on standard RT-PCR with the addition of a third gene-specific oligonucleotide (referred to as a probe) which has a fluorescent dye coupled to its 5′ end (typically 6-FAM) and a quenching dye at the 3′ end (typically TAMRA). When the fluorescently tagged oligonucleotide is intact, the fluorescent signal from the 5′ dye is quenched. As PCR proceeds, the 5′ to 3′ nucleolytic activity of Taq polymerase digests the labeled primer, producing a free nucleotide labeled with 6-FAM, which is now detected as a fluorescent signal. The PCR cycle where fluorescence is first released and detected is directly proportional to the starting amount of the gene of interest in the test sample, thus providing a way of quantitating the initial template concentration. Samples can be internally controlled by the addition of a second set of primers/probe specific for a housekeeping gene such as GAPDH which has been labeled with a different fluorophore on the 5′ end (typically VIC).

Primer sets and probes were designed based on the primary sequences described above, using standard art recognized techniques, for example, PRIMER EXPRESS® (Perkin-Elmer) software.

hAMCase and mAMCase exons 1-3 were amplified using a combination of SEQ ID NO:9 (sense) and SEQ ID NO:10 (antisense) and probe SEQ ID NO:11.

| | |
|---|---|
| 5′-TTCTCCTCACAGGTCTTGTCCTT-3′ | (SEQ ID NO: 9) |
| 5′-CCAGTTGGTGAAGTAGCATGTCA-3′ | (SEQ ID NO: 10) |
| 5′-ATTTGCAGCTCGGCTCTGCCTACCAG-3′ | (SEQ ID NO: 11) |

Human AMCase and monkey AMCase exon 9 were amplified using a combination of SEQ ID NO:12 (sense) and SEQ ID NO:13 (antisense) and probe SEQ ID NO:14.

| | |
|---|---|
| 5′-GCATAAGGCACTTCCTGAGGG-3′ | (SEQ ID NO: 12) |
| 5′-TGACAACATCAAGAGCTTCGA-3′ | (SEQ ID NO: 13) |
| 5′-AGCCAACCCACACATTGCCCTGA-3′ | (SEQ ID NO: 14) |

Human AMCase and monkey AMCase exon 11 were amplified using a combination of SEQ ID NO:15 (sense) and SEQ ID NO:16 (antisense) and probe SEQ ID NO:17.

| | |
|---|---|
| 5′-TTCTGTTATTTGCCACGGGG-3′ | (SEQ ID NO: 15) |
| 5′-CCCGGCCTGGCAGTTC-3′ | (SEQ ID NO: 16) |
| 5′-CCTTCTGGCACTGCGTGAATGGAGT-3′ | (SEQ ID NO: 17) |

The engineered human novel chitinase (or the putative hNC) and macaque novel chitinase exons 1-3 were amplified using a combination of SEQ ID NO:18 (sense) and SEQ ID NO:19 (antisense) and probe SEQ ID NO:20.

(SEQ ID NO: 18)

5′-ACAAGGAGCAGACCAGTGAGG-3′

(SEQ ID NO: 19)

5′-TGACATGTTACTTCACCAACTGGG-3′

(SEQ ID NO: 20)

5′-CTGGTAGGCAGAGCCTATTTCAGCTGTCAG-3′

The engineered human novel chitinase (or the putative hNC) and macaque novel chitinase exon 8 were amplified using a combination of SEQ ID NO:21 (sense) and SEQ ID NO:22 (antisense) and probe SEQ ID NO:23.

| | |
|---|---|
| 5′-CAGGGCCCCAGCTGAGA-3′ | (SEQ ID NO: 21) |
| 5′-GGGTTGCTCAGAAGGAAGGAG-3′ | (SEQ ID NO: 22) |
| 5′-CTCTTGGTTGGATTCCCAGCCTATGGAC-3′ | (SEQ ID NO: 23) |

The engineered human novel chitinase (or the putative hNC) and macaque novel chitinase exon 9 were amplified using a combination of SEQ ID NO:24 (sense) and SEQ ID NO:25 (antisense) and probe SEQ ID NO:26.

| | |
|---|---|
| 5′-TCCTGAAGAATGGAGCTACTGAAGT-3′ | (SEQ ID NO: 24) |
| 5′-CCAAGCCACTCATTTCCTTTG-3′ | (SEQ ID NO: 25) |
| 5′-TGGGAGGCTTCTGAGGATGTTCCCTATG-3′ | (SEQ ID NO: 26) |

qRT-PCR reactions were performed as follows. Total RNA was prepared from a series of human tissues using an RNEASY® kit from Qiagen. First strand cDNA was prepared from 1 mg total RNA using an oligo-dT primer and SUPERSCRIPT™ II reverse transcriptase (Gibco/BRL). cDNA obtained from approximately 50 ng total RNA was used per TAQMAN® reaction.

As shown in FIG. 10A, comparable mRNA expression levels were detected in transfected COS-M6 cells expressing hAMCase (hMCase), human chitotriosidase (hChitotriosidase), the engineered hNC (hNC), and vector. hAMCase (triplicates shown as A, B, and C), engineered hNC, described above (triplicates shown as A, B, and C), and human chitotriosidase were transfected into COS-M6 cells, as follows. Cells were cultured in DME media and transfected at 90% confluency using TransIT®-LT1 transfection reagent (MIRUS). Cells were transfected with constructs encoding hAMCase, human chitotriosidase, hNC (engineered construct), and a vector control. 24 hours post transfection, cell culture media was replaced with serum-free R1CD1 media. Cells were harvested 48 later and RNA was isolated using the Qiagen RNEASY® Mini RNA isolation kit, according to the manufacturer's instruction (Qiagen). qRT-PCR was then performed using a ABI PRISM® 7000 qRT-PCR machine and a combination of SEQ ID NOs: 9, 10, and 11 to amplify hAMCase and mAMCase, and a combination of SEQ ID NOs: 18, 19, and 20 to amplify hNC and mNC.

Endogenous hAMCase, mAMCase, putative hNC, and mNC expression levels were analyzed using qRT-PCR, as follows. RNA was isolated from human stomach, spleen, normal lung and COPD lung, as well as macaque stomach spleen and bronchiole as described above. qRT-PCR reactions were performed using a ABI PRISM® 7000 qRT-PCR machine, as described above. hAMCase and mAMCase exons 1-3 expression was analyzed using SEQ ID NOs: 9, 10, and 11.

As shown in FIG. 10B, a high level of mRNA expression of hAMCase exons 1-3 was detected in human stomach. However, expression of hAMCase exons 1-3 mRNA was not detected in human spleen, normal lung, or COPD lung. Expression of mAMCase exons 1-3 mRNA was not detected in monkey stomach, spleen or bronchiole.

Putative hNC and mNC exons 1-3 expression was analyzed using SEQ ID NOs: 18, 19, and 20. As shown in FIG. 10C, a low level of putative hNC exons 1-3 mRNA expression was detected in human normal lung and COPD lung. Putative hNC exons 1-3 mRNA expression was not detected in human stomach or spleen. In contrast, a high level of mNC exons 1-3 mRNA expression was detected in macaque stomach. mNC exons 1-3 mRNA expression was not detected in macaque spleen or bronchiole.

Putative hNC and mNC exon 8 expression was analyzed using SEQ ID NOs: 21, 22, and 23. As shown in FIG. 10D, consistent with FIG. 10C, hNC mRNA exon 8 expression was detected in human normal lung and COPD lung. Interestingly, primers directed against exon 8 detected significantly higher putative hNC mRNA exon 8 expression levels than those observed using primers directed against exons 1-3, as shown in FIG. 10C. Also consistent with FIG. 10C, hNC exon 8 mRNA expression was not detected in human stomach or spleen. Again consistent with FIG. 10C, a high level of mNC exon 8 mRNA expression was detected in macaque stomach. mNC exon 8 mRNA expression was not detected in macaque spleen or bronchiole.

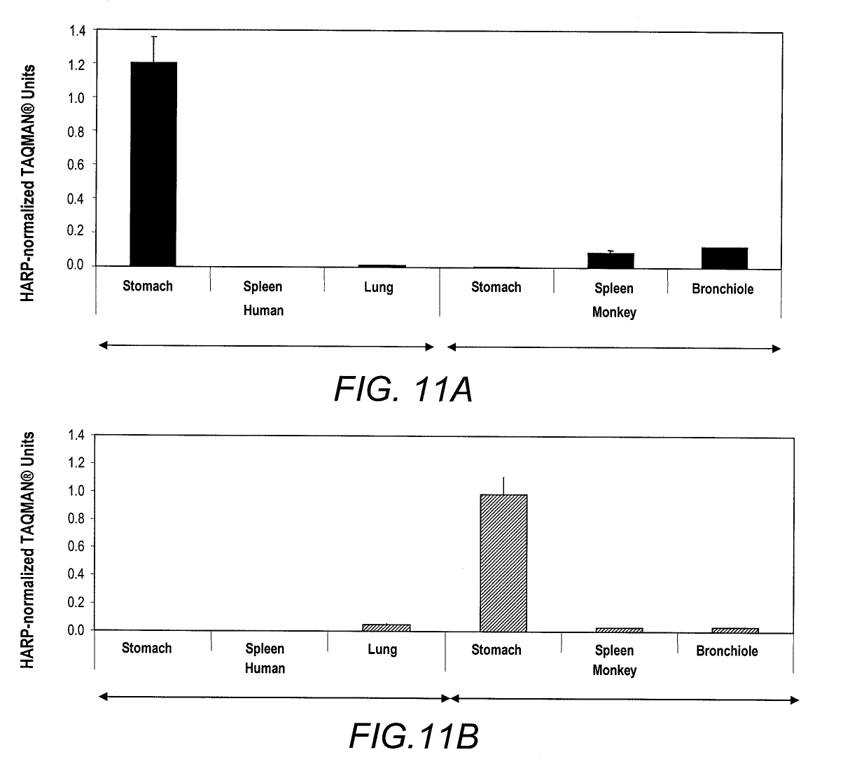

hAMCase and mAMCase exon 11 mRNA expression was analyzed using SEQ ID NOs: 9, 10, and 11. As shown in FIG. 11A, consistent with those data presented in FIG. 10B, hAMCase exon 11 mRNA expression was detectable in human stomach. In contrast, hAMCase exon 11 mRNA expression was not detected in human spleen or lung. mAMCase exon 11 mRNA expression was not detected in macaque stomach. In contrast to mAMCase exons 1-3 (see FIG. 10B), a low level of mAMCase exon 11 mRNA expression was observed in macaque spleen and bronchiole.

Putative hNC and mNC exon 9 expression was analyzed using SEQ ID NOs: 24, 25, and 26. As shown in FIG. 11B, consistent with those data presented in FIGS. 10C and 10D, putative hNC exon 9 mRNA expression was not detectable in human stomach or spleen, however, low level expression was observed in human lung. Also consistent with FIGS. 10C and 10D, a high level of mNC exon 9 mRNA expression was observed in macaque stomach. A low level of mNC exon 9 mRNA expression was observed in macaque spleen and lung.

Together, these qRT-PCR data show that AMCase is expressed in human, but not macaque samples. In contrast, NC is expressed predominantly in monkey. Low level putative hNC expression was observed in human. Interestingly, this low level putative hNC expression was detected most readily using primers designed to target sites in exons 4-11.

As shown in FIGS. 12A-12B, sequence alignment of the above described genes revealed mNC is most homologous to the engineered hNC, with 91.7% and 87.6% homology at the nucleotide and amino acid level, respectively. Alignment of mNC with hAMCase (SEQ ID NO: 29) revealed 84% and 82.1% homology at the nucleotide and amino acid level, respectively (see FIG. 12B). Interestingly, the family 18 catalytic site motif DGXDXDXE (SEQ ID NO:36) (amino acids 133-140 in FIGS. 12A and 12B) was fully conserved in all three genes.

Using the sequence alignment data presented in FIGS. 12A-12B, the gene structure of mNC, mAMCase, and putative hNC was predicted based on previously described hAMCase (SEQ ID NO:29), as shown in FIG. 12C.

Based on the low level of detection of putative hNC (see FIG. 10) and the sequence analysis data presented supra (deletion leading to stop codon), it is believed that the putative hNC is an inactive pseudogene. Likewise, based on the lack of detection of mAMCase expression as sequence analysis data presented supra, it is believed that mAMCase is an inactive pseudogene. Conversely, mNC has an expression profile similar to hAMCase (SEQ ID NO:29). It is, therefore, predicted that mNC may represent a hAMCase functional homologue.

### Example 3

### Cloning and Enzymatic Characterization of Macaque Chitinase

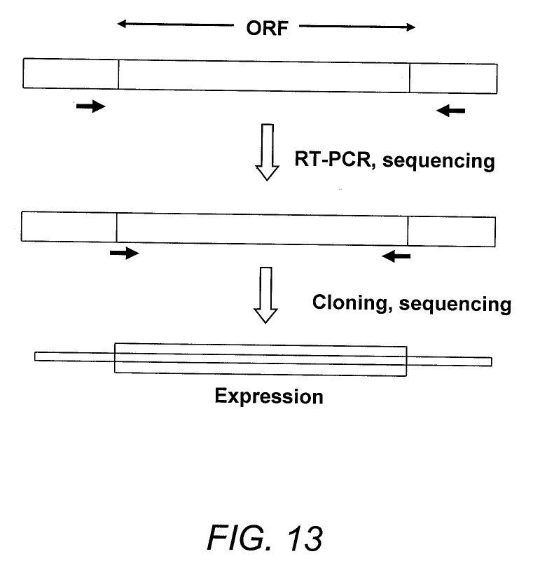

To further investigate the above predictions, mNC was cloned, as follows. Macaque sequences acquired using primers SEQ ID NOs: 5, 6, 7, and 8, were used to design and generate monkey specific primers (designated SEQ ID NO: 27 and SEQ ID NO: 28) to amplify and clone mNC, according to FIG. 13.

(SEQ ID NO: 27)

5′-GGGGACAAGTTTGTACAAAAAAGCAGGCTTCCACCATGGCCAAGCTT
ACCCTCCTCACTG-3′

(SEQ ID NO: 28)

5′-GGGGACCACTTTGTACAAGAAAGCTGGGTCCTAGTGATGGTGATGGT
GATGCCAGCTGCAGCAGGAGCAGAAGGT-3′

Monkey stomach RNA was purified from monkey stomach tissue. “One step” RT-PCR was then performed to generate a cDNA using a one step RT-PCR kit, according to the manufacturer's instructions (Qiagen). Briefly, reactions were performed using SEQ ID NO: 27 and SEQ ID NO:28 and the following conditions. Reverse transcription was performed at 50° C. for 30 minutes. PCR was performed using an initial detnaturization of 95° C. for 15 minutes, and 45 cycles of denaturing at 94° C. for 30 seconds, annealing at 60° C. for 30 seconds, and primer extension at 68° C. for 90 seconds. Sufficient quantities of mNC DNA for cloning purposes were then generated using a second independent PCR, as follows. PCR was performed using the Pfx DNA polymerase kit, according to the manufacturer's instructions (Invitrogen). Briefly, PCR was performed using an initial denaturation of 94° C. for 5 minutes, and 30 cycles of denaturing at 94° C. for 15 seconds, annealing at 60° C. for 15 seconds, primer extension at 60° C. for 15 seconds. A final primer extension was performed at 68° C. for 90 seconds.

mNC DNA was then cloned into a cloning vector using GATEWAY® technology (Invitrogen). Similar attempts to clone hNC from human stomach RNA and mAMCase from monkey stomach were unsuccessful. hAMCase was amplified from human stomach RNA, isolated as described above, by RT-PCR, as described above, using SEQ ID NOs: 27 and 28. The resulting hAMCase DNA was then cloned into a cloning vector using GATEWAY® technology (Invitrogen).

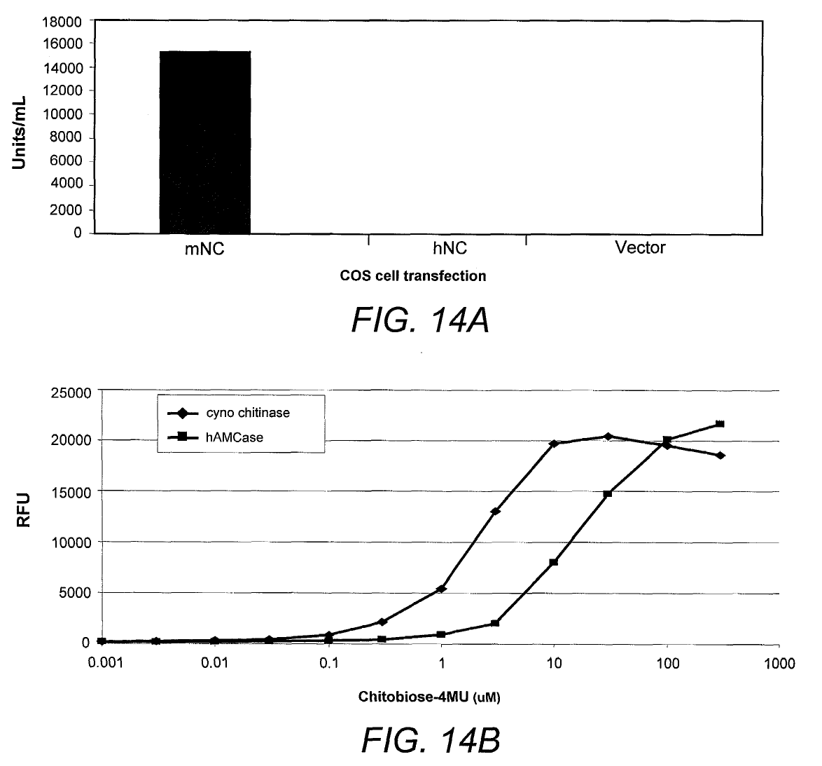

Enzymatic activity assays were performed using cloned mNC, engineered hNC (stop codon substituted to allow for translation of full protein), engineered mut-hNC (truncated), and cloned hAMCase in COS cells. The full length mNC construct was expressed and the chitinase activity of the resultant gene products was measured. As shown in FIG. 14A, mNC shows significant enzymatic activity. In contrast, no significant enzymatic activity was detected for the human chitinase (hNC). These observations suggest that mNC is functionally active in monkeys and may represent the functional analogue of hAMCase. In contrast, hNC appears to be an inactive pseudogene.

Detailed experimental conditions are set forth below.

Cultured conditions were as follows: COS cells were cultured in DME media and were transfected at 90% confluency using TransIT®-LT1 transfection reagent (MIRUS). 24 hours post transfection, cell culture media was replaced with serum-free R1CD1 media. Conditioned media, containing secreted enzyme, was collected 48 hours later. Chitinase enzyme assays were performed directly from these samples, as follows.

hAMCase, engineered with a C-terminal 6-histidine tag, was purified to a single band on SDS-PAGE from a stable CHO cell line transfected with cDNA encoding this hAMCase construct by nickel chromatography. Similarly, mNC, engineered with a C-terminal 6-histidine tag, was purified to a single band on an SDS-PAGE from COS cells transfected with cDNA encoding this mNC construct by nickel chromatography. The enzymatic activity of recombinant hAMCase to mNC was compared, as follows. As shown in FIG. 14B, when each of the above described purified chitinase proteins were exposed to increasing concentrations of chitobiose-4MU, an art-recognized chitinase test substrate, a lower amount of substrate was required to reach one-half Vmax for mNC than hAMCase. This observation indicates that mNC has a lower Km for chitobiose-4MU. More specifically, our calculations revealed that the apparent Km of chitobiose-4MU was 2 μM for mNC and 20 μM for hAMCase.

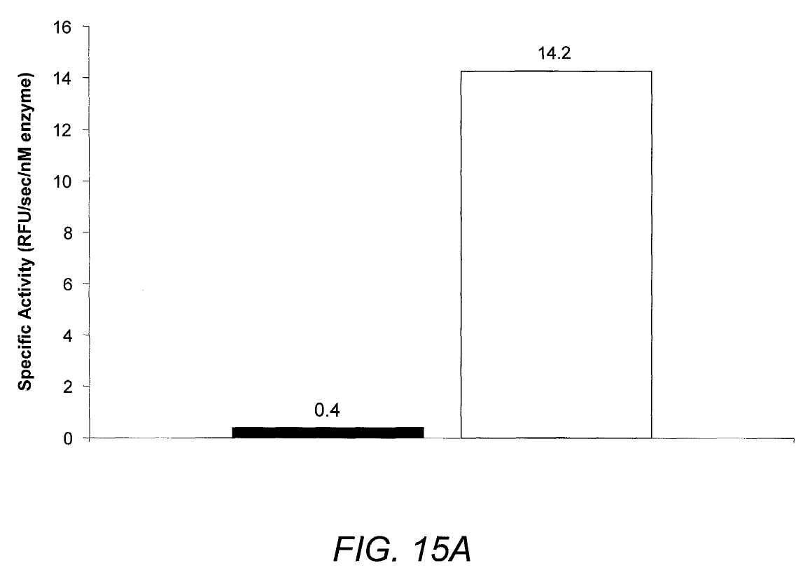

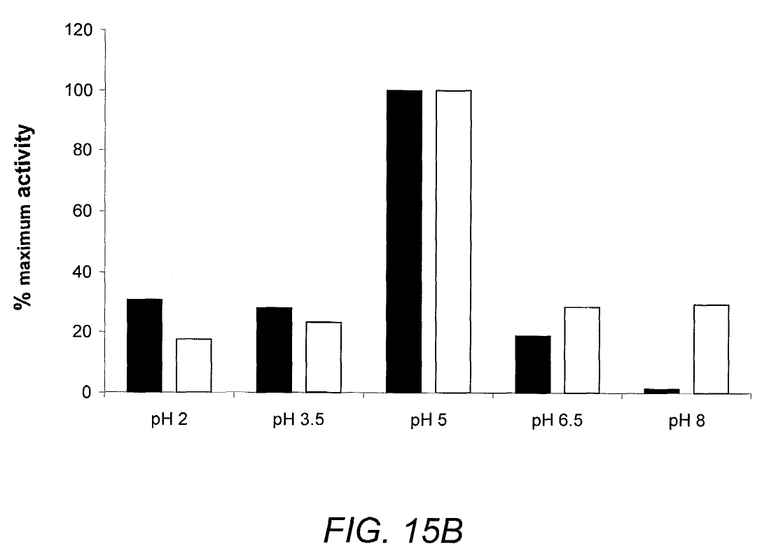

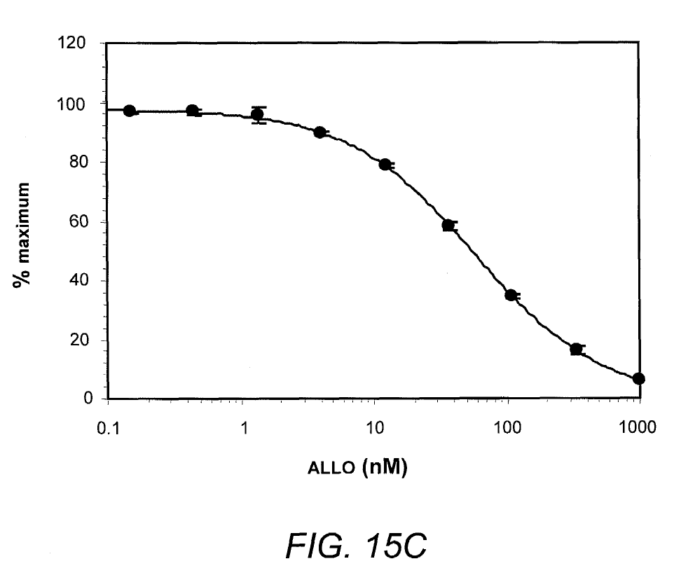

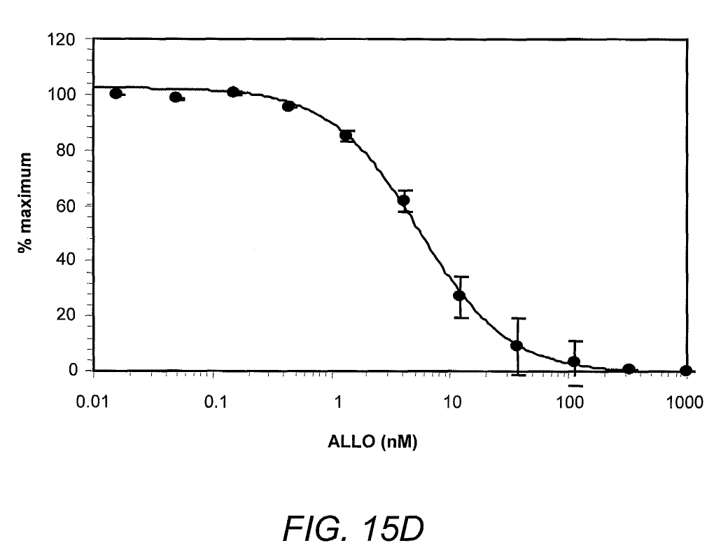

As shown in FIGS. 15A-15D, further characterization of the purified mNC was performed by measuring the relative specific activity, pH profiling, and inhibition by an art-recognized chitinase inhibitor, methylallosamidin. Briefly, various concentrations of mNC, ranging from 0.156 nM to 5 nM, or hAMCase, ranging from 1.56 nM to 50 nM, were incubated with 5 μM chitobiose-4MU substrate in 0.1 M citrate phosphate, 0.0005% Brij35 buffer. The relative specific activity of each enzyme was then determined by measuring the initial rate of enzyme activity versus the enzyme concentration. As shown in FIG. 15A, mNC has a higher relative specific activity (14.4 units/sec/nM) than hAMCase (0.4 units/sec/nM).

Analysis of the pH profiles of hAMCase and mNC were performed by incubating various concentrations of purified hAMCase (ranging from 0.05 nM to 50 nM) or mNC (ranging from 0.018 nM to 5 nM) with 5 μM chitobiose-4MU substrate in 0.1 M citrate phosphate buffer containing 0.005% Brij35 prepared at pH 2 to pH 8. The slope of the initial enzyme rate for each of the enzyme concentrations was then compared at each pH. As shown in FIG. 15B, maximal activity was observed for both mNC and hAMCase at pH 5.0. The slope of the initial rate at the various enzyme concentrations measured for this maximal activity was 13.1 for mNC and 0.45 for hAMCase. This maximal value was set to 100% for each enzyme and the relative activity, for comparison of the activity at different pH values. Both mNC and hAMCase have lower activity, but retain significant activity, at lower pH values of 2.0 and 3.5.

Analysis of hAMCase and mNC inhibition by methylallosamidin was performed by incubating various concentrations of methylallosamidin with 2 nM mNC or 2 nM hAMCase for 10 minutes prior to the addition of 5 μM chitobiose-4MU substrate. Maximal activity was determined by the initial rate of the enzyme activity without the inhibitor methylallosamidin. The percentage of enzyme activity was then calculated and plotted for each concentration of methylallosamidin compared to maximal activity. For the data shown in FIGS. 15C and 15D, the maximal initial rate (RFU/sec) is 1.1 for hAMCase and 21.0 for mNC. There was a dose-dependent inhibition of both enzymes with increasing concentrations of methylallosamidin. The IC₅₀ of methylallosamidin was 58 nM for mNC and 5.5 nM for hAMCase. These data show that methylallosamidin is a more potent inhibitor of hAMCase than mNC. Conversely, 10 times more methylallosaminidin was required to inhibit mNC than hAMCase.

Together, the data presented above suggest that despite high degrees of conservation at both the nucleotide and amino level between hAMCase, hNC, mAMCase, and mNC, hNC is a non-functional pseudogene, even despite our efforts to engineer an active gene based on various other active chitinase sequence, including mNC. mAMCase is also an inactive pseudogene, probably resulting from a stop-codon identified in exon 8. In contrast, although mNC has a higher degree of homology with hNC, which appears to be a pseudogene, it possesses substantial enzymatic activity. The enzymatic activity observed for mNC, however, is substantially different from hAMCase. As described above, when compared to hAMCase, mNC has a 35-fold higher relative specific activity, a 10-fold lower apparent Km for chitobiose-4MU, and requires about a 10-fold higher concentration of methylallosamidin to inhibit maximal enzyme activity by one-half.

In summary, through genome database search, the presence of several primate AMCase-like sequences was identified in human and macaque genomes. In both species, two closely related sequences, AMCase and NC, were detected suggesting duplications of the original gene. The inability to clone functional cDNA for both hNC and mAMCase suggests that both sequences represent inactive or non-functional pseudogenes. A cyno chitinase (mNC) gene was, however, successfully cloned and characterized. This sequence is most homologous to a human pseudogene, designated herein as hNC. In contrast, hAMCase, is most homologous to mAMCase pseudogene in monkeys. Furthermore, although hAMCase and mNC have the same pH profile, mNC has a 35-fold higher relative specific activity, a 10-fold lower apparent Km for chitobiose-4MU, and requires about 10-fold higher concentration of methylallosamidin to inhibit maximal enzyme activity by one-half compared to hAMCase. Based on these differences, it is proposed that mNC originates from a different evolutionary pathway than hAMCase involving a different copy of the chitinase ancestor gene.

### Example 4

### Chitinase k_{cat} and Kₘ Determinations

The k_{cat} and Kₘ values for hAMCase (AMCase) and mNC (cyno chitintase) were determined using a chitobioside-4MU substrate. The ability of hAMCase and mNC to cleave chitin was also determined.

Materials and Methods

Chitin RBV assay. Carboxymethyl-substituted chitin labeled covalently with Remazol Brilliant Violet (CM-Chitin-RBV) (Loewe Biochemica, Sauerlach, Germany) was used to measure chitinolytic activity (Wirth S J, Wolf G A, (1992) Soil Biol Biochem v24 p511-519). A solution of 2 mg/ml CM-chitin-RBV (50 μL) was added to hAMCase (AMCase) or mNC (cyno chitintase) in a total volume of 200 μL of buffer (0.2 M citrate phosphate buffer pH 5.0) in 96-well v-bottom plates (Costar Cat. No. 3894 Acton, Mass.). The plates were sealed with low evaporation lids and incubated at 37° C. for 24 hours. The reaction was terminated by the addition of 1 N HCl, causing precipitation of the non-degraded high-polymeric substrate. The plates were cooled on ice for 10 min. and centrifuged (1400 g, 10 min). The supernatants (100 μL) were transferred to 96-well plates (Costar cat. No. 3690) and measured at 550 nM (Molecular devices).

Results

Kₘ and K_{cat} values for hAMCase and mNC. To better understand the mechanism for the increased specific activity of mNC compared to hAMCase, we measured the kₘ and k_{cat} for each enzyme. The progress curves of hAMCase (1 nM) hydrolysis reactions for 4MU product formation were linear during the first 10 min. for chitobioside-4MU substrate concentrations between 0 to 75 μM (data not shown). The rate of product formation was slower at the highest substrate concentration tested, 150 μM, compared to 75 μM, reflecting substrate inhibition due to transglycosylation, consistent with a previous report (Chou et al. (2006) Biochemistry 45, 4444-4454). We confirmed the high substrate concentration was not due to inner filter effect. In separate control experiments, the 4MU fluorescent signal was not quenched by chitobiose-4MU up to 600 μM. For cyno chitinase (0.25 nM), the hydrolysis reactions for 4MU product formation were linear during the first 10 min. for chitobioside-4MU substrate concentrations between 0 to 20 μM. mNC exhibits apparent transglycosylation at substrate concentrations starting at 37 μM.

The initial rates within the first 10 min. of hAMCase (1 nM) or mNC (0.25 nM) were plotted against the concentration of chitobiose-4MU that were in the linear range for both enzymes. A Michaelis-Menten nonlinear regression fit (Eq 1) K_{m app} values of, 6+/−1 μM for mNC and 42+/−6 μM for AMCase (Table 1). mNC had a higher k_{cat}, 31.8+/−3.2 compared to hAMCase, 3.0+/−0.35, resulting in a higher k_{cat}/Kₘ for mNC compared to hAMCase (5,300,000 vs. 71,429 sec⁻¹/M). The same Kₘ and K_{cat} values for each enzyme were obtained from Line-weaver Burke plots of the linear curves.

TABLE 1
Kinetic parameters for hydrolysis of chitobiose-4MU by hAMCase
and mNC (cyno chitinase). Data were obtained with pH 5.0
citrate phosphate buffer as described in Materials and Methods.

| | Kₘ(μM) | K_{cat}(s − 1) | K_{cat}/Kₘ(s − 1/M) |
|---|---|---|---|
| hAMCase | 42 ± 6 | 3.0 ± .35 | 71,429 |
| mNC | 6 ± 1 | 31.8 ± 3.2 | 5,300,000 |

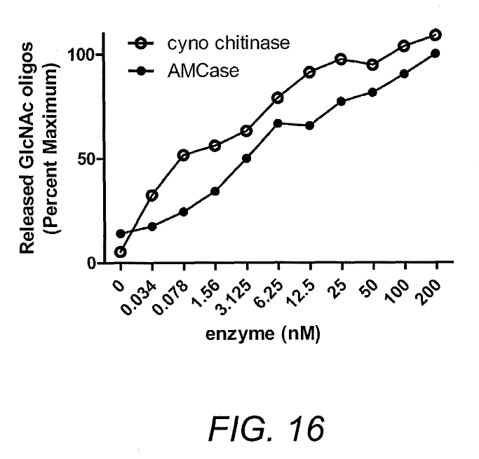

Chitin hydrolysis. We employed the chitin RBV assay, a soluble, dye-labelled and acid-precipitable assay for endo-chitinases to determine if mNC and hAMCase hydrolyze chitin (Wirth S J, Wolf G A (1992) Soil Biol Biochem 24, 511-519). Both hAMCase and mNC generated soluble chitin oligomers after incubation with the Chitin-RBV at 37° C. for 24 hours. The released amount of soluble chitin oligomers was dose-dependent on enzyme concentration (FIG. 16).

The contents of all references, patents and published patent applications cited throughout this application are incorporated herein by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. An isolated nucleic acid molecule comprising a nucleotide sequence selected from the group consisting of:
a) a nucleotide sequence that encodes a pNC polypeptide comprising a chitinase catalytic domain from about amino acid 22 to about amino acid 408 of SEQ ID NO:2, a linker sequence from about amino acid 409 to about amino acid 427 of SEQ ID NO:2, and a chitin-binding domain from about amino acid 428 to about amino acid 474 of SEQ ID NO:2;
b) a nucleotide sequence that encodes a polypeptide comprising the amino acid sequence of SEQ ID NO:2;
c) a nucleotide sequence that encodes a polypeptide comprising an amino acid sequence at least 83% identical to the amino acid sequence of SEQ ID NO:2;
d) a nucleotide sequence comprising the sequence of SEQ ID NO:1;
e) a nucleotide sequence comprising a nucleotide sequence at least 85%, identical to the nucleotide sequence of SEQ ID NO:1;
f) a nucleotide sequence comprising a fragment of at least 100 contiguous nucleotides of SEQ ID NO:1;
g) a nucleotide sequence which hybridizes under highly stringent hybridization conditions to a nucleic acid molecule comprising the nucleotide sequence of SEQ ID NO:1, or a complement thereof; and
h) a nucleotide sequence which comprises nucleotides 513-524, 780-784, 830-835, 843-847, 1117-1124, 1211-1237, 1211-1226, 1220-1237, 1262-1288, 1295-1305, 1312-1320, 1360-1376, 1404-1430, 1404-1414 and 1418-1430, of SEQ ID NO:1.

2. The nucleic acid molecule of claim 1 further comprising vector nucleic acid sequences.

3. The nucleic acid molecule of claim 1 further comprising nucleic acid sequences encoding a heterologous polypeptide.

4. A host cell which contains the nucleic acid molecule of claim 1.

5. The host cell of claim 4 which is a mammalian host cell.

6. A non-human mammalian host cell containing the nucleic acid molecule of claim 1.

7. An isolated polypeptide selected from the group consisting of:
a) a pNC polypeptide comprising a chitinase catalytic domain from about amino acid22 to about amino acid 408 of SEQ ID NO:2, a linker sequence from about amino acid409 to about amino acid 427 of SEQ ID NO:2, and a chitin-binding domain from about amino acid428 to about amino acid 474 of SEQ ID NO:2;
b) a polypeptide comprising the amino acid sequence of SEQ ID NO:2;
c) a polypeptide comprising an amino acid sequence at least 83% identical to the amino acid sequence of SEQ ID NO:2;
d) a polypeptide comprising an amino acid sequence encoded by a nucleotide sequence comprising the sequence of SEQ ID NO:1;
e) a polypeptide comprising an amino acid sequence encoded by a nucleotide sequence comprising a nucleotide sequence at least 85% identical to the nucleotide sequence of SEQ ID NO:1;
f) a polypeptide comprising an amino acid sequence encoded by a nucleotide sequence comprising a fragment of at least 100 contiguous nucleotides of SEQ ID NO:1;
g) a polypeptide comprising an amino acid sequence encoded by a nucleotide sequence which hybridizes under highly stringent hybridization conditions to a nucleic acid molecule comprising the nucleotide sequence of SEQ ID NO:1, or a complement thereof;
h) a fragment of a pNC polypeptide of at least 90 contiguous amino acids of SEQ ID NO:2; and
i) a fragment of a pNC polypeptide comprising about amino acids 22-408, 409-427, 428-474, or about amino acids 1-21, 22-120, 22-180, 22-240, 22-300, 22-360, 22-400, 120-150, 150-170, 170-180, 180-190, 190-220, 220-250, 250-270, 270-280, 280-290, 290-300, 300-320, 320-330, 330-340, 340-350, 350-360, 360-390, 390-400, 400-410, 410-420, 420-430, 430-440, 440-450, 450-460, 460-470, or 470-476, of SEQ ID NO:2.

8. The polypeptide of claim 7 further comprising heterologous amino acid sequences.

9. A method for producing a pNC polypeptide, comprising culturing the host cell of claim 4 under conditions in which the nucleic acid molecule is expressed.

10. An antibody molecule which selectively binds to a polypeptide of claim 7, wherein said antibody molecule binds to human AMCase with an affinity that is less than 30% compared to the affinity of the antibody molecule to the pNC.

11. An antibody molecule with binds to a polypeptide of claim 7 and human AMCase with substantially equal affinities.

12. The antibody molecule of claim 10 or 11, which can reduce one or more activities of a pNC polypeptide.

13. A method of providing an antibody molecule that specifically binds to a pNC polypeptide, comprising:
(i) providing a non-human chitinase, or fragment thereof, wherein said non-human chitinase is at least 85% identical to a corresponding portion of a human chitinase protein;
(ii) obtaining an antibody molecule that specifically binds to the non-human chitinase or fragment thereof; and
(iii) evaluating if the antibody molecule specifically binds to the human chitinase protein.

14. The method of claim 13, further comprising administering the antibody molecule to a subject.

15. The method of claim 13, wherein the subject is a human or non-human primate.

16. A method of evaluating the ability of a test compound to interact with a pNC polypeptide or nucleic acid, comprising:
contacting a pNC polypeptide or nucleic acid with the test compound; and
evaluating a change in one or more activities of the pNC polypeptide or nucleic acid in the presence of the test compound, relative to a control sample without the test compound.

17. The method of claim 16, wherein the activities evaluated comprise a change in the expression, binding, or enzymatic activity of the pNC polypeptide or nucleic acid.

18. The method of claim 16, wherein the contacting step is effected in vitro or in vivo.

19. A method for identifying a test compound, which inhibits the activity of a pNC polypeptide, or the expression of a pNC nucleic acid, comprising: contacting the pNC polypeptide or nucleic acid with a test compound; and determining the effect of the test compound on the activity of the polypeptide or nucleic acid to thereby identify a compound which inhibits the activity of the polypeptide or nucleic acid.

20. The method of claim 19, wherein the test compound is a peptide, a phosphopeptide, a small molecule, a nucleic acid, an antisense molecule, a ribozyme, an RNAi, a triple helix molecule, an antibody molecule, a chitinase inhibitor or an analogue thereof, or any combination thereof.

21. The method of claim 19, wherein the test compound is an inhibitor of chitinase activity.

22. The method of claim 19, wherein the contacting step is effected by administering the test compound to a subject, and evaluating a change in one or more symptoms of a chitinase-associated disorder.

23. The method of claim 22, wherein the symptoms evaluated comprise one or more of: (i) detecting a change in the number of eosinophils, macrophages, or neutrophils into the airways; (ii) measuring eotaxin levels; (iii) detecting in basophil histamine release; or (iv) detecting IgE titers, wherein a reduction, in the level of one or more of (i)-(iv) comparing before and after treatment) indicates that the test compound is effectively reducing airway eosinophilia in the subject.

24. An inhibitor of chitinase activity identified by the method of claim 19.
